SEQUENCE LISTING (36 sequences)

SEQ ID NO: 1 - DNA, 21 bp, synthetic construct
  source 1..21: mol_type = other DNA
ctcagttcctacgcttcgcat

SEQ ID NO: 2 - DNA, 20 bp, synthetic construct
  source 1..20: mol_type = other DNA
gtcgaggtcagtgaacagca

SEQ ID NO: 3 - DNA, 20 bp, synthetic construct
  source 1..20: mol_type = other DNA
ttcgtgtgcaagcctgagat

SEQ ID NO: 4 - DNA, 20 bp, synthetic construct
  source 1..20: mol_type = other DNA
taatataccgcggagctggc

SEQ ID NO: 5 - DNA, 21 bp, synthetic construct
  source 1..21: mol_type = other DNA
tggcagtaccccatgtctgaa

SEQ ID NO: 6 - DNA, 21 bp, synthetic construct
  source 1..21: mol_type = other DNA
ccaagaccgtcacaaaaaggc

SEQ ID NO: 7 - DNA, 21 bp, synthetic construct
  source 1..21: mol_type = other DNA
cgacaccccaatctcgatatg

SEQ ID NO: 8 - DNA, 21 bp, synthetic construct
  source 1..21: mol_type = other DNA
gttgcacagatagtgacccgt

SEQ ID NO: 9 - DNA, 21 bp, synthetic construct
  source 1..21: mol_type = other DNA
acagtgcccttcagccagact

SEQ ID NO: 10 - DNA, 25 bp, synthetic construct
  source 1..25: mol_type = other DNA
acaactactttttcacagccttcgt

SEQ ID NO: 11 - DNA, 21 bp, synthetic construct
  source 1..21: mol_type = other DNA
gggatgctgttgaaaggtgaa

SEQ ID NO: 12 - DNA, 20 bp, synthetic construct
  source 1..20: mol_type = other DNA
ggtggtccggtaaatcctgg

SEQ ID NO: 13 - DNA, 20 bp, synthetic construct
  source 1..20: mol_type = other DNA
gagctttgcaggaagtttgc

SEQ ID NO: 14 - DNA, 20 bp, synthetic construct
  source 1..20: mol_type = other DNA
gcaagaagcctctccttgaa

SEQ ID NO: 15 - DNA, 20 bp, synthetic construct
  source 1..20: mol_type = other DNA
aggttgggattcgtgtctgg

SEQ ID NO: 16 - DNA, 23 bp, synthetic construct
  source 1..23: mol_type = other DNA
agttgtttgtagttctcaggcag

SEQ ID NO: 17 - DNA, 19 bp, synthetic construct
  source 1..19: mol_type = other DNA
gcccctcattaagcccaag

SEQ ID NO: 18 - DNA, 21 bp, synthetic construct
  source 1..21: mol_type = other DNA
ttgtggtggtctgacagttcg

SEQ ID NO: 19 - DNA, 21 bp, synthetic construct
  source 1..21: mol_type = other DNA
tgcacgatcagacagtcattc

SEQ ID NO: 20 - DNA, 22 bp, synthetic construct
  source 1..22: mol_type = other DNA
gtagagctgaggagcgacaatc

SEQ ID NO: 21 - DNA, 21 bp, synthetic construct
  source 1..21: mol_type = other DNA
actgacggagagcatgaagat

SEQ ID NO: 22 - DNA, 19 bp, synthetic construct
  source 1..19: mol_type = other DNA
ccggcacataggaggggta

SEQ ID NO: 23 - DNA, 21 bp, synthetic construct
  source 1..21: mol_type = other DNA
aggtcaatctccctacacgag

SEQ ID NO: 24 - DNA, 22 bp, synthetic construct
  source 1..22: mol_type = other DNA
ggagtaaggatcttgcgactgg

SEQ ID NO: 25 - DNA, 20 bp, synthetic construct
  source 1..20: mol_type = other DNA
acaacgatggcctctacgac

SEQ ID NO: 26 - DNA, 19 bp, synthetic construct
  source 1..19: mol_type = other DNA
agttcacgcaccagcacac

SEQ ID NO: 27 - DNA, 20 bp, synthetic construct
  source 1..20: mol_type = other DNA
acctggagatgcagatcgaa

SEQ ID NO: 28 - DNA, 20 bp, synthetic construct
  source 1..20: mol_type = other DNA
aatccacctccacactgacc

SEQ ID NO: 29 - DNA, 21 bp, synthetic construct
  source 1..21: mol_type = other DNA
tttctcaggcccaattttcct

SEQ ID NO: 30 - DNA, 21 bp, synthetic construct
  source 1..21: mol_type = other DNA
gctgtcggaggtatatggtgg

SEQ ID NO: 31 - DNA, 23 bp, synthetic construct
  source 1..23: mol_type = other DNA
gcggagtgtaatcagtatttgga

SEQ ID NO: 32 - DNA, 21 bp, synthetic construct
  source 1..21: mol_type = other DNA
gcatttgatcccgtacaacct

SEQ ID NO: 33 - DNA, 56 bp, synthetic construct
  source 1..56: mol_type = other DNA
ggggacaagtttgtacaaaaaagcaggcttcaccatgttgtggaaaataaccgata

SEQ ID NO: 34 - DNA, 52 bp, synthetic construct
  source 1..52: mol_type = other DNA
ggggaccactttgtacaagaaagctgggtcttatttcctgtgtttctccatt

SEQ ID NO: 35 - DNA, 51 bp, synthetic construct
  source 1..51: mol_type = other DNA
ggggacaagtttgtacaaaaaagcaggcttcaccatgagcagcccggatgc

SEQ ID NO: 36 - DNA, 52 bp, synthetic construct
  source 1..52: mol_type = other DNA
ggggaccactttgtacaagaaagctgggtctcacacgtcaggatagttgcag

(19) United States
(12) Patent Application Publication
(10) Pub. No.: US 2024/0101957 A1
(43) Pub. Date: Mar. 28, 2024

(54) METHODS AND COMPOSITIONS FOR GENERATING EMBRYOS IN VITRO FROM PLURIPOTENT STEM CELLS

(71) Applicants: California Institute of Technology, Pasadena, CA (US); Cambridge Enterprise Limited, Cambridge (GB)

(72) Inventors: Magdalena Zernicka-Goetz, Pasadena, CA (US); Bailey A.T. Weatherbee, Cambridge (GB); Carlos W. Gantner, Cambridge (GB)

(21) Appl. No.: 18/460,022

(22) Filed: Sep. 1, 2023

Related U.S. Application Data
(60) Provisional application No. 63/403,684, filed on Sep. 2, 2022.
(60) Provisional application No. 63/457,670, filed on Apr. 6, 2023.

(51) Int. Cl. C12N 5/073; C12M 1/32; C12N 5/00; G01N 33/50

(52) U.S. Cl. CPC C12N 5/0604; C12M 23/12; C12N 5/0031; G01N 33/5044; C12N 2501/60; C12N 2502/02

(57) ABSTRACT

Disclosed herein include methods, compositions and culture media for generating synthetic embryos in vitro from mammalian pluripotent stem cells such as pluripotent embryonic stem cells. In some embodiments, the method can comprise co-culturing a wild-type mammalian pluripotent stem cell and modified mammalian pluripotent stem cells comprising one or more genes encoding transcription factors that can drive generation of extraembryonic cells or extraembryonic-like cells (e.g., GATA6 gene, SOX17 gene, GATA3 gene and/or TFAP2C gene) under a condition in a culture medium allowing the pluripotent stem cells to self-organize into a post-implantation embryo structure. In some embodiments, the pluripotent embryonic stem cells are human pluripotent embryonic stem cells and the generated synthetic embryo is a human embryo.

### RELATED APPLICATIONS

This application claims the benefit under 35 U.S.C. § 119(e) of U.S. Provisional Patent Application No. 63/403,684, filed Sep. 2, 2022 and U.S. Provisional Patent Application No. 63/457,670, filed Apr. 6, 2023, the contents of these related applications are incorporated herein by reference in their entirety for all purposes.

### REFERENCE TO SEQUENCE LISTING

The present application is being filed along with a Sequence Listing in electronic format. The Sequence Listing is provided as a file entitled 30KJ-302455-WO-SeqList, created Aug. 24, 2023, which is 33 kilobytes in size. The information in the electronic format of the Sequence Listing is incorporated herein by reference in its entirety.

### BACKGROUND

### Field

The present disclosure relates generally to the field of cell culture, in particular, culturing embryos and stem cells.

### Description of the Related Art

The mammalian embryo, particularly human embryo, undergoes morphogenetic transformations following implantation into the uterus, yet knowledge of this crucial stage is limited by the inability to observe the embryo in vivo. Stem cell-derived models of the embryo are important tools to interrogate developmental events and tissue-tissue crosstalk during these stages.

### SUMMARY

Disclosed herein includes an in vitro method of generating a mammalian synthetic embryo. The method, in some embodiments, comprises co-culturing a wild-type mammalian embryonic stem cell (ESC), a first modified mammalian ESC comprising GATA6 gene and/or SOX17 gene, and a second modified mammalian ESC comprising GATA3 gene and/or TFAP2C gene under a condition in a culture medium allowing the ESCs to self-organize into a post-implantation embryo structure. In some embodiments, the first modified mammalian ESC comprises an inducible GATA6 gene, an inducible SOX17 gene, or both. In some embodiments, the first modified mammalian ESC comprises an inducible GATA6 gene and an inducible SOX17 gene. In some embodiments, the second modified mammalian ESC comprises an inducible GATA3 gene, an inducible TFAP2C gene, or both. In some embodiments, the second modified mammalian ESC comprises an inducible GATA3 gene and an inducible TFAP2C gene.

The method can, in some embodiments, further comprises contacting the first modified mammalian ESC and/or the second modified mammalian ESC with an inducer. The inducer can be, for example, doxycycline. In some embodiments, the inducer is supplied to the culture medium, optionally for a duration of about 1-7 day. The method can, in some embodiments, further comprises modulating the strength of the induction, optionally by increasing or decreasing the concentration of the inducer or increasing or decreasing of the duration of the inducer in the culture medium. In some embodiments, the inducer is supplied to the culture medium during the entire co-culturing process. In some embodiments, the wild type mammalian ESC and/or the modified mammalian ESCs are naïve ESCs or primed ESCs. In some embodiments, the wild type mammalian ESC and/or the modified mammalian ESCs are pre-implantation naïve hESCs, peri-implantation-like pluripotent naïve hESCs, or post-implantation primed hESCs. In some embodiments, the pre-implantation naïve hESCs are cultured in PXGL medium prior to the co-culturing, the peri-implantation-like pluripotent hESCs are cultured in RSeT medium prior to the co-culturing, and the post-implantation-like primed hESCs are cultured in mTeSR1 medium prior to the co-culturing. In some embodiments, the wild type mammalian ESC and the modified mammalian ESCs are peri-implantation-like pluripotent hESCs, optionally cultured in RSeT medium prior to the co-culturing. In some embodiments, the wild type mammalian ESC, the first modified mammalian ESC comprising GATA6 and/or SOX17 gene, and the second modified mammalian ESC comprising GATA3 and/or TFAP2C gene are provided at a ratio from about 1:1:1 to 1:1:5, optionally at a ratio from about 1:1:1 to 1:1:2. In some embodiments, the ESCs are cultured in a substrate, optionally wherein the substrate comprises a dish, a U-plate, a flask or a microwell plate. In some embodiments, the ESCs are cultured in inverted pyramidal microwells. In some embodiments, one or more of, or each of, the inverted-pyramidal microwells is about 400 μm or about 800 μm in size, optionally about 400 μm or about 800 μm diameter. In some embodiments, the co-culturing comprises co-culturing the ESCs in a stem-cell proliferation medium for about 5 days, optionally passaging the ESCs in the stem-cell proliferation medium at least two times. In some embodiments, the stem-cell proliferation medium is a serum-free medium. In some embodiments, the stem-cell proliferation medium comprises Dulbecco's Modified Eagle Media (DMEM), DMEM Nutrient Mixture 12 (DMEM/F12), neurobasal, N2, B27, L-glutamine or an analogue thereof, a reducing agent, an antibiotic, or a combination thereof. The reducing agent can be, or can comprise, beta-mercaptoethanol (BME), N-acetyl-L-cysteine, dithiothreitol (DTT), or any combination thereof.

In some embodiments, the stem-cell proliferation medium is a N2B27 medium. In some embodiments, the N2B27 medium comprises DMEM/F12, Neurobasal, B27, N2, GlutaMax, β-mercaptoethanol, penicillin/streptomycin or a combination thereof. In some embodiments, the N2B27 medium comprises 1:1 DMEM/F12 and Neurobasal A, 0.5×B27, 0.5×N2, 100 μM β-mercaptoethanol, 1× GlutaMAX, and 1× penicillin-streptomycin.

In some embodiments, the ESCs aggregate following up to 24 hours of co-culturing in the stem-cell proliferation medium. In some embodiments, the aggregated ESCs exhibit distinctions between inner and outer cellular domains. In some embodiments, the co-culturing comprises co-culturing the ESCs in a post-implantation culture medium for at least 2 days, following co-culturing in the stem-cell proliferation medium. In some embodiments, co-culturing the ESCs in the post-implantation culture medium begins about 2 days post-aggregation of the ESCs. In some embodiments, the post-implantation culture medium comprises Dulbecco's Modified Eagle Media (DMEM), DMEM Nutrient Mixture 12 (DMEM/F12), a non-human serum or serum substitute thereof, an antibiotic, an antimicrobial agent, L-glutamine or an analogue thereof, an insulin, an insulin analogue, or an insulin receptor agonist, an estrogen analogue, or an estrogen receptor agonist, progesterone, a progesterone analogue, or a progesterone receptor agonist, or any combination thereof. In some embodiments, the non-human serum or serum substitute comprises fetal bovine serum, bovine serum albumin, KnockOut™ Serum Replacement, or any combination thereof. In some embodiments, the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. The estrogen receptor agonist can be, or can comprise, for example, β-estradiol, estrone, estriol and estetrol, or any analogue thereof.

In some embodiments, the insulin receptor agonist is selected from the group comprising IGF-I, IGF-II, analogues thereof, or any combination thereof. In some embodiments, the post-implantation culture medium comprises an antimicrobial agent, optionally the antimicrobial agent is sodium lactate. In some embodiments, the post-implantation medium comprises transferrin, sodium selenium, ethanolamine, or any analogue thereof. In some embodiments, the post-implantation culture medium comprises DMEM/F12, fetal bovine serum, GlutaMax, non-essential amino acids, essential amino acids, Insulin-Transferrin-Selenium-Ethanolamine (ITS-X), penicillin and/or streptomycin, Glucose, sodium lactate, β-estrodiol, progesterone, or any combination thereof.

In some embodiments, the post-implantation culture medium comprises DMEM/F12, about 20% fetal bovine serum, about 1× GlutaMax, about 1× non-essential amino acids, about 1× essential amino acids, about 1×ITS-X, about 25 U/mL penicillin and/or streptomycin, about 1.8 nM Glucose, about 0.22% sodium lactate, about 8 nM β-estrodiol, about 200 ng/ml progesterone, or any combination thereof. In some embodiments, the co-culturing comprises transferring the ESCs from one substrate to another substrate. In some embodiments, the post-implantation embryo structure comprises an inner epiblast-like domain, a single outer layer of trophoblast-like cells, and an intermediate hypoblast-like domain between the epiblast-like domain and the single outer layer of trophoblast-like cells. In some embodiments, the inner epiblast-like domain is SOX2 positive and contains a central lumen, the single outer layer of trophoblast-like cells is GATA3 positive, and the intermediate hypoblast-like domain is GATA6 positive. In some embodiments, the post-implantation embryo structure expresses N-Cadherin and SOX17 in the hypoblast-like domain, CDX2 in the trophoblast-like cells, and/or SOX2, NANOG and E-Cadherin in the epiblast-like domain. In some embodiments, the inner epiblast-like domain exhibits a pluripotent and epithelial identity akin to a human embryo.

In some embodiments, the post-implantation embryo structure comprises cell clusters resembling embryonic late-epiblast, amnion, mesoderm, extraembryonic mesenchyme, and/or hypoblast/visceral endoderm. In some embodiments, the post-implantation embryo structure expresses TDGF1, SOX2, NANOG, TFAP2A, ID1, ISL1, TFAP2C, VTCN1, GRHL1, MEIS1, TBXT, MESP1, MIXL1, CER1, SNAI1, EOMES, POSTN, COL6A3, IGF2, TBX20, BMP6, CDH2, HNF1B, FOXA2, or a combination thereof. In some embodiments, the post-implantation embryo structure generates amnion and primordial germ cells. In some embodiments, the efficiency of forming an post-implantation embryo from the wild-type mammalian ESC, the first modified mammalian ESC comprising GATA6 gene and/or SOX17 gene, and the second modified mammalian ESC comprising GATA3 gene and/or TFAP2C gene is greater than 5%, 10%, 15%, 20%, 25%, 30%, 35%, or higher.

In some embodiments, the method does not comprise any in vivo step. In some embodiments, none of the wild-type mammalian ESC, the first modified mammalian ESC comprising GATA6 gene and/or SOX17 gene, and a second modified mammalian ESC comprising GATA3 gene and/or TFAP2C gene is present in an in vivo environment during the co-culturing and optionally wherein the in vivo environment comprises a tissue, an organ, an organism, or a combination thereof. In some embodiments, the method does not comprise culturing trophoblast stem cells, hypoblast stem cells or both, alone or in combination with the ESCs. In some embodiments, the wild-type mammalian ESC, the first modified mammalian ESC comprising GATA6 gene and/or SOX17 gene, and the second modified mammalian ESC comprising GATA3 gene and/or TFAP2C gene are human ESCs. In some embodiments, the post-implantation embryo structure is a human embryo structure. In some embodiments, the post-implantation embryo structure resembles a post-implantation human embryo at about 8-9 days post-fertilization. In some embodiments, the method does not comprise the use of an exogenous signaling pathway factor, optionally the culture medium does not comprise or is supplied with the exogenous signaling pathway factor. In some embodiments, the exogenous signaling factor comprises a WNT signaling pathway activator, a TGFβ superfamily member, or both.

Disclosed herein includes a synthetic embryo obtained by any of the methods disclosed herein. In some embodiments, the synthetic embryo is a human embryo, optionally, the synthetic embryo resembles a post-implantation human embryo at about 8-9 days post-fertilization.

Also disclosed herein includes a method of investigating mechanisms involved in embryogenesis, comprising any of the methods disclosed herein. Disclosed herein includes a method of identifying a compound useful for treating a disease, comprising contacting a synthetic embryo obtainable by any of the in vitro methods disclosed herein with the compound.

Also disclosed herein includes a method for diagnosing or treating a disease or disorder in a subject. The method, in some embodiments, comprises generating a synthetic embryo according to any of the methods disclosed herein; and transplanting the synthetic embryo into the subject. In some embodiments, the wild-type mammalian ESC and the modified mammalian ESCs are obtained from the subject or derived from ESCs obtained from the subject.

Also disclosed herein includes a method of elucidating the role of a candidate gene in embryo development, comprising: obtaining a wild-type mammalian ESC, a first modified mammalian ESC comprising GATA6 gene and/or SOX17 gene, and a second modified mammalian ESC comprising GATA3 gene and/or TFAP2C gene, where the candidate gene has been modified or knocked out; and culturing the mammalian ESCs using any of the in vitro methods disclosed herein.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIG. 1A-FIG. 1F depict non-limiting exemplary embodiments and data related to the validation of extraembryonic-like induction. FIG. 1A depicts generation of inducible GATA6 (iG6) and/or SOX17 (iS17) hESCs and validation after 24 hours of doxycycline addition in basal N2B27. iG6 (n=551), iS17 (n=550), and iG6-S17 (n=707) cells from 3 independent experiments. FIG. 1B depicts generation of inducible GATA3 (iG3) and/or AP2γ (iAP2γ) hESCs and validation after 24 hours of doxycycline addition in basal N2B27. iG3 (n=1456), iAP2γ (n=1456) and iG3-AP2γ (n=782) cells from 3 independent experiments. FIG. 1C depicts Uniform Manifold Approximation and Projection (UMAP)-based dimensional reduction of sequenced wildtype (RSeT WT), inducible GATA6-SOX17 (Day 3 iG6-S17) and inducible GATA3-AP2γ (Day 3 iG3-AP2γ) RSeT hESCs after 3 days of doxycycline induction. FIG. 1D depicts logistic regression analysis and comparison of cells to human post-implantation embryo populations. Human embryo data from a previous report was used as training data and cell line data was used as test data. FIG. 1E depicts selected differentially expressed genes from RNA-sequencing (left) and predicted differential motif accessibility from ATAC-sequencing scored by chromVAR (right) for wildtype, inducible GATA6-SOX17, and inducible GATA3-AP2γ RSeT hESCs after 3 days of doxycycline induction. FIG. 1F depicts validation of wildtype, inducible GATA6-SOX17 and inducible GATA3-AP2γ RSeT hESC co-culture in 2D (N=3 independent experiments). In FIG. 1A, FIG. 1B and FIG. 1F, scale bars=100 μm. For FIG. 1A-FIG. 1B, mean±SEM is plotted.

FIG. 2A-FIG. 2H depict non-limiting exemplary embodiments and data related to generation of inducible post-implantation human embryoids. FIG. 2A depicts overview of protocol to generate inducible human embryoids by combining wildtype RSeT hESC with inducible GATA6-SOX17 (iG6-S17) and inducible GATA3-AP2γ (iG3-AP2γ) cells. Extraembryonic-like cells were induced for 3 days before aggregation at Day 0. FIG. 2B shows that at 96 hours post-aggregation, structures demonstrated clear self-organization. FIG. 2C depicts the size of cell aggregates between Days 1-3 post-aggregation. Day 1 (n=175), Day 2 (n=171) and Day 3 (n=91) structures were from 5 independent experiments. All individual embryoid lengths are plotted. Each symbol (orange cross, orange triangle, green triangle, blue circle, purple square) represents an independent experiment. FIG. 2D depicts quantification of embryoid formation across starting pluripotency states. RSeT (n=952), mTeSR (n=30), PXGL (n=207) structures from 5 independent experiments. One-way ANOVA with Holm-Sidak's multiple comparisons test was used for statistical analysis, suggesting RSeT versus mTeSR P=0.0347, and RSeT vs PXGL P=0.0283. Unmarked pairwise comparisons were not significant (n.s.; p>0.05). FIG. 2E depicts quantification of cell type proportions in correctly organized embryoids. N=16 embryoids from 3 independent experiments. FIG. 2F depicts representative images of an in vitro cultured human embryo 9 days post-fertilization, showing clear lumenized SOX2 domain surrounded by a layer of GATA6-positive cells. A subset of GATA6-positive cells expressed the anterior hypoblast marker CER1. The images are representative of 3 independent experiments. FIG. 2G depicts the hypoblast-like domain expressing N-Cadherin, SOX17, and GATA4 and epiblast-like domain maintaining expression of pluripotency factors SOX2, OCT4 and NANOG. Cells derived from inducible GATA3-AP2γ expressing GFP showed clear outer localization. The images are representative of 2 experiments each. FIG. 2H depicts inducible human embryoids demonstrating clear apicobasal polarity, with quantification of inducible human organization. In the top right panel of FIG. 2H, 1 is lumen, 2 is ECM, and 3 is SOX2+lumen+ECM. n=506 structures were from 3 independent experiments for lumen and ECM efficiency. N=27 embryoids were from 2 independent experiments for lumen number. In FIG. 2A, FIG. 2B, and FIG. 2F-FIG. 2H, scale bars=100 μm. * indicates P<0.05. In FIG. 2C, all individual embryoids lengths are plotted. For FIG. 2D-FIG. 2E and FIG. 2H, mean±SEM is plotted. Inner domains of embryoids are surrounded by a dashed line.

FIG. 3A-FIG. 3I depict non-limiting exemplary embodiments and data related to differentiation of extraembryonic mesenchyme, amnion, and primordial germ cells. FIG. 3A depicts schematic of extended culture protocol of inducible human embryoids and sampling for combined single cell RNA and single cell ATAC sequencing using the 10× platform. 12 embryoids each on Days 4, 6, and 8 post-aggregation were sequenced. FIG. 3B depicts cell annotation based on transcriptional projection to multiple human and non-human primate embryo datasets using scmap in conjunction with RNA and chromatin velocity. FIG. 3C depicts selected differentially expressed genes in the RNA-sequencing data (top) and predicted differentially accessible motifs scored by chromVAR on the ATAC-sequencing data (bottom) across clusters. FIG. 3D depicts inducible human embryoids downregulated SOX2 and upregulated CDX2, ISL1 on Day 6 and VTCN1 on Day 8, indicative of robust amnion differentiation and maturation. In some rare cases, dorsoventral and/or anterior-posterior axis patterning was observable. The images are representative of 3 experiments. FIG. 3E depicts module scoring for primordial germ cell marker genes. FIG. 3F depicts Nebulosa plot visualizing joint expression density of key primordial germ cell genes in inducible human embryoids. FIG. 3G depicts heatmap of selected primordial germ cell gene expression across clusters. FIG. 3H depicts quantification of SOX17/NANOG/AP2γ triple-positive (+) cells on days 4 (n=10 embryoids) and 6 (n=10 embryoids). N=2 independent experiments were carried out. FIG. 3I depicts immunofluorescence identification of SOX17/NANOG/AP2γ triple-positive primordial germ cell-like cells in inducible human embryoids highlighted by arrowheads. Mean±SEM is plotted. Scale bars=100 μm. Inner domains of embryoids are surrounded by a dashed line.

FIG. 4A-FIG. 4H depict non-limiting exemplary embodiments and data related to BMP signaling driving amnion specification in inducible human embryoids. FIG. 4A depicts expression of ID1-4, a downstream target of BMP signaling, in embryoids. FIG. 4B depicts chromVAR-based motif accessibility scores of SMAD5 and SMAD2::SMAD3::SMAD4, effectors of BMP and NODAL signaling, respectively. FIG. 4C depicts representative images and quantification of OCT4-positive and GATA6-positive cells from representative inducible human embryoids on Days 4 (n=60 cells each; P<0.0001) and 6 (n=40 cells each; P<0.0001) from N=3 independent experiments. FIG. 4D depicts representative images and quantification of SMAD2.3 in OCT4-positive and GATA6-positive cells from representative inducible human embryoids on Days 4 (n=40 cells each; P=0.0004) and 6 (n=40 cells each; P<0.0001) from n=2 independent experiments. FIG. 4E demonstrates that inhibition of BMP signaling blocks exit from pluripotency and upregulation of amnion markers AP2a and CDX2. FIG. 4F depicts quantification of the percentage of Day 4 inner domains expressing SOX2 and CDX2 (control: n=147; LDN-treated: n=126; BMP4-treated: n=57; and Act-A-treated: n=60 embryoids from 5 independent experiments). For SOX2+/CDX2-domains, Control vs LDN P=0.0002, Control vs Act-A P=0.0433, Control vs BMP4 P=0.1753. FIG. 4G demonstrates that inhibition of BMP reduces the number of primordial germ cell-like cells in embryoids. FIG. 4H depicts quantification of the number of SOX17/NANOG/AP2γ triple-positive primordial germ cell-like cells (PGCLC) on Day 4 (control: n=45; LDN-treated: n=30; BMP4-treated: n=48; and Act-A-treated: n=36 embryoids from 6 independent experiments). Control vs LDN P=0.0011. Control vs BMP4 P>0.99. Control vs Act-A P=0.98. Unmarked comparisons to control are n.s. Statistics used in FIG. 4C-FIG. 4D was two-sided Mann-Whitney; in FIG. 4F was RM two-way ANOVA with Holm-Sidak's multiple comparison's test; and in FIG. 4H was Kruskal-Wallis with Dunn's multiple comparisons test. Scale bars=100 μm. In FIG. 4C-FIG. 4D and FIG. 4F, mean±SEM is plotted. For FIG. 4H, box encompasses the 25^{th}-75^{th} quartiles with whiskers to the minimum and maximum. Central line denotes median and + symbol denotes mean. *P<0.05, **P<0.01, ***P<0.001, ****P<0.0001. Inner domains of embryoids are surrounded by a dashed line.

FIG. 5A-FIG. 5H depict non-limiting exemplary embodiments and data related to the antagonistic effect of SOX17 induction to specification of the anterior hypoblast. FIG. 5A depicts expression of CER1 and LEFTY1 in the HYPO/VE in embryoids. FIG. 5B depicts analysis of GATA6 and SOX17 regulon activity scored by SCENIC and SOX17 and CER1 co-expression in post-implantation human hypoblast (9-11 days post-fertilization). Data was from a previous report. FIG. 5C depicts representative examples of embryoids showing CER1-positive cells generated using induced GATA6 (iG6) but not induced GATA6-SOX17 (iG6-S17) dual induction of hypoblast-like cells. CER1 expression was also observed if doxycycline was withdrawn on Day 3 post-aggregation. FIG. 5D depicts representative images on Day 4, demonstrating decreased pSMAD1.5 in the epiblast-like domain of structures with a CER1-positive cell population. FIG. 5E depicts the proportion of embryoids in FIG. 5C expressing CER1. iG6-S17: n=78; iG6: n=25; iS17: n=26; iG6-S17-Dox. day 1: n=54; and iG6-S17-Dox. day 3: n=65 embryoids were from 7 independent experiments. iG6-S17 vs iG6 P<0.0001. iG6-S17 v iG6-S17-Dox. day 3 P<0.0001. iG6-S17 vs iS17 P=0.87. iG6-S17 vs iG6-S17-Dox. day 1 P=0.87. FIG. 5F depicts quantification of pSMAD1.5 levels in SOX2-positive cells in CER1-negative (CER1−) versus CER1-positive (CER1+) iG6-S17 embryoids on Day 4. CER1−: n=108 cells; and CER1+: n=123 cells from 8 embryoids each from 2 independent experiments. In FIG. 5F, P<0.0001. FIG. 5G depicts quantification of Brachyury expression in FIG. 5D. FIG. 5H depicts representative images of BRY/TBXT expression in inducible human embryoids generated with iG6, iS17, or iG6-S17 cells (with doxycycline maintained, removed at day 1 or day 3 post-aggregation). iG6-S17: n=34; iG6: n=15; iS17: n=16; iG6-S17-Dox. day 1: n=16; and iG6-S17-Dox. day 3: n=20 embryoids from 6 independent experiments. iG6-S17 vs iG6 P=0.0225. iG6-S17 vs iG6-S17-Dox. day 3 P=0.0002. iG6-S17 vs iS17 P=0.69. iG6-S17 vs iG6-S17-Dox. day 1 P=0.81. Statistics used in FIG. 5E and FIG. 5G was RM two-way ANOVA with Holm-Sidak's multiple comparisons test; in FIG. 5F was two-way Mann Whitney test. Scale bars=100 μm. For FIG. 5E, FIG. 5F and FIG. 5G, mean±SEM is plotted. *P<0.05, **P<0.01, ***P<0.001, ****P<0.0001. Inner domains of embryoids are surrounded by a dashed line.

FIG. 6A-FIG. 6H depict non-limiting exemplary embodiments and data related to the selection of transgenes to drive extraembryonic-like cells. FIG. 6A depicts Uniform Manifold Approximation Projection (UMAP) showing combined human pre- to post-implantation datasets colored by original publications. FIG. 6B depicts UMAP of combined human datasets colored according to stage of embryo (d.p.f. is short for days post-fertilization). FIG. 6C depicts UMAP of combined human datasets colored according to cell type. FIG. 6D depicts cardinal cell type gene expression on UMAP of human datasets. FIG. 6E depicts plots from single cell RNA sequencing of key marker gene expression in human datasets separated by cell type (n=10223 cells). FIG. 6F depicts inferred epiblast, hypoblast, and trophoblast gene regulatory network generated by SCENIC during peri-implantation human embryo development. Candidate factors are marked with boxes (TFAP2C, GATA3, GATA6, and SOX17). FIG. 6G depicts regulon activity scored by SCENIC for hypoblast markers GATA6, SOX17, and TrB markers GATA3 and TFAP2C (n=10223 cells). FIG. 6H depicts qRT-PCR analysis of individual inducible cell lines. Doxycycline inducible constructs were inserted in Shef6 hESC using piggybac transposase (inducible GATA6, SOX17, GATA6-SOX17, GATA3, AP2γ and GATA3-AP2γ). Colonies were manually isolated after single cell plating and propagated. Appropriate transgene expression was validated by RT-qPCR after 72 hours of 1 μg/mL doxycycline addition in basal N2B27 conditions. N=3 technical replicates were included. Clones selected for further analysis were marked with boxes. For box plots, box encompasses the 25th-75th quartile with whiskers to minimum and maximum, with central line marking median.

FIG. 7A-FIG. 7F depict non-limiting exemplary embodiments and data related to immunofluorescence analysis of cardinal marker genes of hypoblast and trophoblast after doxycycline induction across pluripotent states. FIG. 7A depicts qRT-PCR analysis after 3 days of doxycycline-induction of induced GATA6 (iG6), induced SOX17 (iS17), or induced GATA6-SOX17 (iG6-S17) singly and together from three pluripotent states. FIG. 7B depicts qRT-PCR analysis after 3 days of DOX-induction of induced GATA3 (iG3), induced AP2γ (iAY), or induced GATA3-AP2γ (iG3-AY) from multiple pluripotent starting states. For FIG. 7A-FIG. 7B, n=3 technical replicates were from 3 independent experiments. FIG. 7C depicts immunofluorescence analysis of iG6, iS17, or iG6-S17 cells after 3 days induction from multiple pluripotent states. FIG. 7D depicts quantification of immunofluorescence levels of FIG. 7C. FIG. 7E depicts immunofluorescence analysis of iG3, iAY, or iG3-AY after 3 days induction from multiple pluripotent states. FIG. 7F depicts quantification of immunofluorescence levels of FIG. 7E. For FIG. 7C-FIG. 7F, n=3 technical replicates were from 2 independent experiments. Cells were initially cultured in either mTeSR, RSeT, or PXGL conditions, and then cultured for 3 days either under the same conditions or alternatively transferred to either basal N2B27 media or basal N2B27 media with the addition of doxycycline. The induced transgenes are marked with box. Scale bars=100 μm.

FIG. 8A-FIG. 8D depict non-limiting exemplary embodiments and data related to the comparison of transcription factor-mediated induction with published directed differentiation methods. FIG. 8A depicts comparison and quantification of GATA6, SOX17 and SOX2 after yolk sac-like cell (Activin-A, CHIR99021 and LIF) directed differentiation, doxycycline-mediated induction in inducible GATA6-SOX17 cells, or both. Cells were differentiated from RSeT conditions. The figure legend of the chart in the bottom panel of FIG. 8A is the same as in FIG. 8B. FIG. 8B depicts comparison and quantification of EOMES, N-Cadherin, and OTX2 after yolk sac-like cell (Activin-A, CHIR99021, and LIF) directed differentiation, doxycycline-mediated induction in inducible GATA6-SOX17 cells, or both. For FIG. 8A-FIG. 8B, N2B27: n=717; ACL: n=1211; N2B27+Dox.: n=522; and ACL+Dox.: n=544 cells were from 3 fields of view obtained from 2 independent experiments. FIG. 8C depicts comparison and quantification of GATA3, AP2a, and SOX2 after PA (PD0325901 and A83-01) or PAL (PD0325901, A83-01, and LPA) directed differentiation, doxycycline-mediated induction in inducible GATA3-AP2γ cells, or both. The figure legend of the chart in the bottom panel of FIG. 8C is the same as in FIG. 8D. FIG. 8D depicts comparison and quantification of GATA2, KRT7, and AP2γ after PA (PD0325901 and A83-01) or PAL (PD0325901, A83-01, and LPA) directed differentiation, doxycycline-mediated induction in inducible GATA3-AP2γ RseT cells, or both. For FIG. 8C-FIG. 8D, N2B27: n=443; PA: n=487; PAL: n=371; N2B27+Dox.: n=357; PA+Dox.: n=412; and PAL+Dox.: n=287 cells were from 3 fields of view obtained from 2 independent experiments. Scale bars=100 μm. For FIG. 8A-FIG. 8D, mean±SEM is plotted. Differentiation was carried out on hESC in RSeT conditions.

FIG. 9A-FIG. 9G depict non-limiting exemplary embodiments and data related to assessing extraembryonic-like induction from RseT cells. FIG. 9A depicts quality control plots of cell line 10× multiome sequencing data (n=5328 cells). Violin plots go from minimum to maximum. FIG. 9B depicts logistic regression framework to assess similarity between clusters, which was applied to cell line RNA-sequencing data using published in vitro blastoid and directed differentiation protocols as training data. FIG. 9C depicts gene expression of selected genes after 3 days of doxycycline-induction from sequencing of wildtype, inducible GATA6-SOX17 (iG6-S17), and inducible GATA3-AP2γ (iG3-AP2γ) RSeT hESC populations visualized on a uniform manifold projection and approximation (UMAP). Visualization of sample distribution in the UMAP is shown in FIG. 1C. FIG. 9D depicts immunofluorescence images of human cell-mouse embryo chimeras at the late blastocyst stage, showing shift of human cells marked by human nuclear antigen (HuNAg) contributing to the SOX2-positive epiblast to the SOX17-positive primitive endoderm upon iG6-S17 induction. FIG. 9E depicts quantification of the contribution of HuNAg-positive cells stained for SOX2 and SOX17. Control: n=12 and iG6-S17: n=30 embryos from 3 independent experiments. FIG. 9F depicts immunofluorescence images of human cell-mouse embryo chimeras at the late blastocyst stage, showing a shift of human cells from the SOX2-positive epiblast to the GATA3-positive trophectoderm upon iG3-AP2γ induction. FIG. 9G depicts quantification of the contribution of HuNAg-positive cells stained for SOX2 and GATA3. Control: n=10 and iG6-AP2γ: n=27 embryos from 3 independent experiments. Induction was carried out from hESC in RSeT conditions. For box plots, box encompasses the 25^{th}-75^{th} quartile with whiskers to minimum and maximum, and with central line marking median. Scale bars=100 m.

FIG. 10A-FIG. 10F depict non-limiting exemplary embodiments and data related to post-implantation human embryo-like model cluster identification. FIG. 10A depicts Day 4 embryoids generated from a second hESC line, RUES2. N=371 structures were from 2 independent experiments. FIG. 10B depicts brightfield images of inducible human embryoids selected on Days 4, 6, and 8 for sequencing (n=12 at each stage). Note the presence of an inner domain surrounded by two concentric domains. FIG. 10C depicts quality control plots for embryoid sequencing data on Days 4, 6, and 8 post-aggregation (n=5217 cells). Violin plots go from minimum to maximum. FIG. 10D depicts scmap projection of inducible human embryoid cells onto cynomolgus macaque (M. fasicularis) and human datasets (H. sapien) spanning peri-implantation to gastrulation stages. FIG. 10E depicts cardinal marker gene expression for epiblast, endoderm and mesoderm, and trophoblast and amnion within the stem cell-derived model. FIG. 10F depicts alluvial plot showing the contribution of Day 4, 6, or 8 embryoids to assigned cell type. Scale bars=100 μm. Inner domains of embryoids are surrounded by a dashed line.

FIG. 11A-FIG. 11D depict non-limiting exemplary embodiments and data related to embryoid cluster comparison to human and cynomolgus monkey datasets. FIG. 11A depicts logistic regression analysis comparing annotated clusters from cynomolgus macaque (M. fasicularis) and human datasets (H. sapiens) spanning peri-implantation to gastrulation stages (training data) to post-implantation human embryo-like model clusters (test data). Cynomolgus data was from previous reports. FIG. 11B depicts logistic regression analysis comparing in vitro human embryo-like model and directed differentiation datasets (training data) to inducible human embryoids (test data). FIG. 11C depicts scmap projection of human inducible embryoid dataset onto in vitro datasets. In vitro datasets were from 3 previous reports. FIG. 11D depicts violin plots of gene expression in GFP-negative versus GFP-positive cells derived from induced GATA3-AP2γ (iG3-AP2γ) cells from inducible human embryoid sequencing dataset (n=5217 cells). Violin plots go from minimum to maximum.

FIG. 12A-FIG. 12J depict non-limiting exemplary embodiments and data related to extraembryonic mesenchyme trajectory and wildtype cell differentiation capacity. FIG. 12A depicts immunofluorescence of HAND1 demonstrating expression in GATA6-positive cells (putative extraembryonic mesenchyme) and upregulation between days 4 and 6 in putative amnion (AP2γ-positive). The images are representative of 2 experiments. FIG. 12B depicts expression of HAND1 in the inducible human embryoid single cell sequencing dataset. FIG. 12C depicts immunofluorescence of TBX20 demonstrating high expression in a subset of GATA6-positive cells (putative extraembryonic mesenchyme). The images are representative of 5 experiments. FIG. 12D depicts expression of TBX20 in the inducible human embryoid single cell sequencing dataset demonstrating enrichment in the extraembryonic mesenchyme cluster. FIG. 12E depicts differentiation of ISL1-positive amnion and GATA6/TBX20-positive extraembryonic mesenchyme in structures derived from a second cell background, RUES2. The images are representative of 2 experiments. FIG. 12F depicts differentiation of primordial germ cell-like cells in embryoids derived from a second cell background, RUES2. The images are representative of 2 experiments. FIG. 12G depicts examples and quantification of day 4 embryoids. Embryoids exhibited an outer layer of GFP-positive induced GATA3-AP2γ (iG3-AP2γ) cells, an inner domain comprised of mKate2-positive wildtype hESCs, and an interstitial GATA6-positive population largely comprised of unlabeled induced GATA6-SOX17 (iG6-S17) cells. N=9 embryoids were from 2 independent experiments. FIG. 12H depicts ISL1-positive amnion-like cells overlapping with mKate2-positive wildtype cells. The images are representative of 3 experiments. FIG. 12I depicts expression of GATA6 and TBX20-positive extraembryonic mesenchyme-like cells overlapping with mKate2-positive wildtype cells. The images are representative of 3 experiments. FIG. 12J depicts expression of AP2γ, SOX17, and NANOG triple-positive primordial germ cell-like cells overlapping with mKate2-positive wildtype cells. The images are representative of 3 experiments. Scale bars=100 μm. For FIG. 12G, mean±SEM is plotted. Inner domains of embryoids are surrounded by a dashed line.

FIG. 13A-FIG. 13H depict non-limiting exemplary embodiments and data related to the role of BMP and inducible GATA3-AP2γ cells in generating inducible human embryoids. FIG. 13A depicts expression of ID1-4 fitting over a latent time, colored by cell type assignment. FIG. 13B depicts motif accessibility scored by chromVAR for SMAD5 and SMAD2::SMAD3::SMAD4 fitting over latent time, colored by cell type assignment. FIG. 13C depicts predicted ligand-receptor pairings in inducible human embryoids generated by CellPhoneDB. FIG. 13D depicts predicted interactions of inducible GATA6-SOX17 (G6-S17) and inducible GATA3-AP2γ (G3-AP2γ) cells after 3 days of induction with wildtype RseT hESCs, which are the cell types aggregated to generate inducible human embryoids. FIG. 13E depicts inducible human embryoids failing to form if induced GATA3-AP2γ cells are excluded or if the BMP signaling antagonist LDN193189 (LDN) is added between days 0-2. FIG. 13F depicts quantification of embryoid formation efficiency from FIG. 13E. N=535 ESC+iG6-S17 and 500 LDN-treated structures were from 4 independent experiments. FIG. 13G depicts quantification of embryoid size after LDN193189 addition between Days 0-2. N=105 structures per condition for each day were from 3 independent experiments. Statistics test used was two-sided Mann Whitney between Control and LDN at each timepoint (Day 1 P<0.0001, Day 2 P=0.0019, and Day 3 P<0.0001). FIG. 13H depicts overview of whole Aggrewells demonstrating the effect of BMP inhibition, BMP4 addition, and NODAL activation. The images are representative of 5 experiments. Note the significant increase in well-organized structures expressing SOX2 after BMP inhibition. Scale bars=100 μm. **P<0.01. ****P<0.0001. For FIG. 13F, mean±SEM is plotted. For FIG. 13G, all individual datapoints are plotted.

FIG. 14A-FIG. 14C depict non-limiting exemplary embodiments and data related to the downregulation of CER1 expression upon extended culture of inducible human embryoids. FIG. 14A depicts formation efficiency of embryoids generated with different conditions. Note the highest efficiency is the standard condition described herein with consistent addition of doxycycline and using GATA6-SOX17 inducible cells (iG6-S17). iG6-S17: n=224; induced GATA6 (iG6): n=276; induced SOX17 (iS17): n=247; iG6-S17 with doxycycline removed at day 1: n=370; and iG6-S17 with doxycycline removed at day 3: n=410 structures from 2 independent experiments. FIG. 14B depicts immunofluorescence of CER1 and SOX2 on Day 6 post-aggregation, demonstrating downregulation of both SOX2 and CER1 at this stage in structures generated with both inducible iG6 or iG6-S17 hypoblast-like cells (with consistent addition or doxycycline, or early removal at day 3 post-aggregation) together with wildtype ESCs and inducible GATA3-AP2γ (iG3-AP2γ) cells. The images are representative of 3 experiments. FIG. 14C shows that embryoids generated with Shef6-mKate2 ESCs demonstrate that both the ISL1-positive and BRY-positive cell populations differentiate from the wildtype cells in structures generated with either iG6 or iG6-S17 hypoblast-like cells with early removal of doxycycline at day 3 post-aggregation. In FIG. 14C, the images show DAPI, mKate2 (WT ESC), ISL1, BRY, and merged view of mKate2, ISL1, and BRY from top to bottom. The images are representative of 3 experiments. Scale bars=100 μm. Inner domains of embryoids are surrounded by a dashed line.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

The term “about,” as used herein when referring to a measurable value such as an amount of a compound, dose, time, temperature, and the like, is meant to encompass variations of 10%, 5%, 1%, 0.5%, or even 0.1% of a specified amount.

The term “stem cell” as used herein can refer to a cell capable of retaining a constant potential for differentiation even after cell division. Examples of stem cells include: embryonic stem cells with pluripotency derived from a fertilized egg or clone embryo; epiblast stem cells; trophoblast stem cells; extraembryonic endoderm (XEN) stem cells; somatic stem cells and pluripotent stem cells that are present in tissues in a living organism e.g. hepatic stem cells, dermal stem cells, and reproductive stem cells that serve as the bases for respective tissues; pluripotent stem cells derived from reproductive stem cells; pluripotent stem cells obtained by nuclear reprogrammed somatic cells; totipotent stem cells and non-totipotent stem cells and the like.

The term “pluripotent stem cell” (PSC) as used herein refers to a stem cell permitting in vitro culture and having the potential for differentiating into all cells, but the placenta. The pluripotent stem cell has the potential to differentiate into any of the three germ layers: endoderm (which forms structures such as the gastrointestinal tract and the respiratory system), mesoderm (which forms structures such as the musculoskeletal system, the vascular system and the urogenital system), or ectoderm (which forms epidermal tissues and the nervous system).

The term “embryonic stem cell” (ES cell or ESC) as used herein refers to a pluripotent stem cell derived from the inner cell mass of a blastocyst, which is an early-stage preimplantation embryo. It is envisaged that such cells may express genes involved in the naïve pluripotency network (Oct4/Nanog, Sox2, Klf4 etc). Such cells may also have Oct4 proximal enhancer activity. They may contribute to all embryonic tissues in chimeras. The ES cells may be derived from mammalian embryos, obtained from iPS cells or obtained from appropriate cell lines. Non-limiting examples of said stem cells include embryonic stem cells of a mammal or the like established by culturing a pre-implantation early embryo, embryonic stem cells established by culturing an early embryo prepared by nuclear-transplanting the nucleus of a somatic cell, induced pluripotent stem cells (iPS cells) established by transferring several different transcriptional factors to a somatic cell, and pluripotent stem cells prepared by modifying a gene on a chromosome of embryonic stem cells or iPS cells using a gene engineering technique. More specifically, embryonic stem cells include embryonic stem cells established from an inner cell mass that constitutes an early embryo, embryonic stem cells established from a primordial germ cell, cells isolated from a cell population possessing the pluripotency of pre-implantation early embryos (for example, primordial ectoderm), and cells obtained by culturing these cells.

The term “trophoblast stem cell” as used herein refers to stem cells derived from the trophoblast lineage of the embryo. The trophoblast stem cells are preferably extra-embryonic cells derived from the two cell types which are precursors of the human placenta: the cytotrophoblast and the syncitiotrophoblast. These cells can be derived at late pre-implantation stages or early post-implantation stages but the resulting cell lines are equivalent to the stem cell compartment existing in the extra-embryonic ectoderm of the post-implantation mouse egg cylinder. Transcription factors such as Cdx2, Tead4, Gata3, Elf5, Eomes, and Tfap2C mark this lineage. TS cells can also be considered as cells that are the precursors of the differentiated cells of the placenta. In the mouse, TS cells can be derived from outgrowths of either blastocyst polar trophectoderm or extraembryonic ectoderm, which originates from polar trophectoderm after implantation.

The term “extra-embryonic endoderm stem cell” (XEN stem cell) as used herein refers to stem cells derived from the extraembryonic endoderm of an embryo. The extraembryonic endoderm is typically a derivative of the hypoblast cells that migrate into the blastocyst cavity (beginning on day 8 of human embryonic development), and line the cavity, giving rise to the primary and definitive yolk sacs. The extraembryonic endoderm fills the remaining cavity of the blastocyst.

As used herein, the term “differentiation” can refer to the process by which an unspecialized (“uncommitted”) or less specialized cell acquires the features of a specialized cell such as, for example, a neuronal cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. As used herein, a “lineage-specific marker” can refer to a characteristic specifically associated with the phenotype of cells of a lineage of interest and can be used to assess the differentiation of an uncommitted cell to the lineage of interest.

As used herein, “markers”, “lineage markers” or, “lineage-specific markers” can refer to nucleic acid or polypeptide molecules that are differentially expressed in a cell of interest. Differential expression can mean an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art. In some embodiments, a marker can be enriched. The term “enriched”, as used herein, shall have its ordinary meaning, and can also refer to a statistically significant increase in levels of a gene product (e.g., mRNA and/or protein) in one condition as compared to another condition (e.g., in one cell layer as compared to another cell layer).

The term “concentration” as used herein shall have its ordinary meaning, and can also refer to (a) mass concentration, molar concentration, volume concentration, mass fraction, molar fraction or volume fraction, or (b) a ratio of the mass or volume of one component in a mixture or solution to the mass or volume of another component in the mixture or solution (e.g., ng/ml). In some embodiments, the concentration can refer to fraction of activity units per volume (e.g., U/ml).

The term “analogue” as used herein refers to a compound which may be structurally related to the relevant molecule. The term “agonist” as used herein can refer to a compound which might not be structurally related to the relevant molecule. For example, an agonist may activate the relevant receptor by altering the conformation of the receptor. Nevertheless, in both cases the terms are used in this specification to refer to compounds or molecules which can mimic, reproduce or otherwise generally substitute for the specific biological activity of the relevant molecule.

As used herein the phrase “culture medium” refers to a liquid substance used to support the growth and development of stem cells and of an embryo. The culture medium used according to some embodiments of the invention can be a water-based medium which includes a combination of substances such as salts, nutrients, minerals, vitamins, amino acids, nucleic acids, and/or proteins such as cytokines, growth factors and hormones needed for cell growth and embryo development.

Provided herein includes methods, compositions and culture media for generating a mammalian (e.g., human) post-implantation embryo model comprised of embryonic and extraembryonic tissues. Two type of extraembryonic-like cells generated by transcription factor overexpression are combined with wild-type embryonic stem cells and self-organize into structures that mimic aspects of a post-implantation embryo such as a post-implantation human embryo. The self-organized structure contains a pluripotent epiblast-like domain surrounded by hypoblast- and trophoblast-like tissues.

Disclosed herein includes an in vitro method of generating a synthetic embryo from embryonic stem cells. In some embodiments, the method comprises co-culturing a wild-type mammalian embryonic stem cell (ESC), a first modified mammalian ESC comprising GATA6 gene and/or SOX17 gene, and a second modified mammalian ESC comprising GATA3 gene and/or TFAP2C gene under a condition in a culture medium allowing the ESCs to self-organize into a post-implantation embryo structure. Disclosed herein also includes a synthetic embryo structure obtained by the method disclosed herein.

Disclosed herein also includes a method of investigating mechanisms involved in embryogenesis according to the in vitro methods disclosed herein. Disclosed herein also includes a method of identifying a compound useful for treating a disease, comprising contacting a synthetic embryo obtainable by the in vitro method disclosed herein with the compound. Disclosed herein also includes a method for diagnosing or treating a disease or disorder in a subject. The method can comprise generating a synthetic embryo according to the method disclosed herein and transplanting the synthetic embryo into the subject. Disclosed herein also includes a method of elucidating the role of a candidate gene in embryo development. The method can comprise obtaining a wild-type mammalian ESC, a first modified mammalian ESC comprising GATA6 gene and/or SOX17 gene, and a second modified mammalian ESC comprising GATA3 gene and/or TFAP2C gene, where the candidate gene has been modified or knocked out; and culturing the mammalian ESCs using the in vitro method disclosed herein.

Generating Synthetic Embryo Structures from Pluripotent Stem Cells

Human reproduction is remarkably inefficient, with an estimated 60% of pregnancies failing during the first two weeks following fertilization. Since the advent of in vitro fertilization, human embryos have been studied throughout the first week of development. However, the second week, which includes implantation into the uterus and preparation for gastrulation, remains a ‘black box’. The human blastocyst at 5-6 days post-fertilization consists of the outermost trophectoderm, precursor of the placenta, and inner cell mass, which gives rise to both the embryonic epiblast and the yolk sac precursor, the hypoblast. Between 7-8 days post-fertilization the blastocyst implants into the endometrium and the epiblast polarizes and transitions from the naïve state of pluripotency to the primed state. A central amniotic cavity forms within the epiblast, separating the dorsal amniotic epithelium and the ventral epiblast, which maintains pluripotency and gives rise to the embryo proper. The trophectoderm develops into several trophoblast subtypes following implantation and the hypoblast forms the primary, and then secondary, yolk sac. A subset of cells in the hypoblast maintains expression of NODAL, BMP and WNT inhibitors, safeguarding the future anterior epiblast from posteriorizing signals during primitive streak formation, marked by upregulation of BRY/TBXT. An additional extraembryonic tissue, the extraembryonic mesenchyme, is located between the inner cell mass-derived tissues and the trophoblast, however, the origin of these cells remains unclear.

Recent work in mouse embryos established conditions amenable to human embryo in vitro culture through implantation, opening this developmental black box for the first time. This system has been used to characterize major developmental events, including formation of the anterior hypoblast domain, specification of trophoblast subtypes and transitions in epiblast pluripotency state. However, mechanistic work in the human embryo remains challenging. Thus, stem cell-derived models of the human embryo will serve as an important and complementary tool to understanding this crucial period of development. Several groups have reported the generation of blastocyst-like structures derived from human embryonic stem cells (hESCs). These blastoids resemble the pre-implantation embryo but develop poorly to post-implantation stages. Other models, including gastruloids, 2D micropatterns and embryoid bodies, are capable of modelling aspects of post-implantation development. However, these models are derived entirely from hESC, lack extraembryonic tissues, and do not recapitulate embryo morphology. A recent study, which combines epiblast-like spheroids with BMP4-treated hESCs expressing a mixture of extraembryonic markers, marks a step towards integrated models of the post-implantation embryo. However, this model does not exhibit self-organization of an epiblast-like compartment in the context of extra-embryonic tissues until after lumenogenesis, and the BMP4 treated hESCs are not correlated to targeted extraembryonic lineages.

Several protocols have been developed to derive trophoblast and hypoblast cells from hESCs. Importantly, the pluripotent state influences the developmental trajectory of differentiated cells. The derivation of lineage-specific cell lines offers scope for modelling these tissues in vitro. However, generating a modular, integrated model system that includes both embryonic and extraembryonic tissues has proven challenging. This may be due to the opposing signaling pathway modulators required for in hESC culture, hypoblast-like cell differentiation, and trophoblast-like cell differentiation. Moreover, while tissue-tissue crosstalk is an advantage of integrated model systems, generation of embryoids in media containing exogenous factors may compromise tissue-driven self-organization.

To overcome these limitations, the present disclosure provides methods, compositions and culture media for generating a synthetic embryo (e.g., a human embryo) from pluripotent stem cells such as pluripotent embryonic stem cells (“ESCs”) based on the approach of expressing (e.g., overexpressing) transcription factors that can drive generation of extraembryonic-like cells, including trophoblast-like cells and hypoblast-like cells, from pluripotent embryonic stem cells. The pluripotent stem cell-based in vitro embryo model described herein is generated with embryonic and extra-embryonic lineages using exclusively pluripotent embryonic stem cells (e.g., human ESCs or “hESCs”). The extra-embryonic trophoblast-like cells and hypoblast-like cells can be generated by transcription factor overexpression with wild type embryonic stem cells. The present disclosure demonstrates that aggregates of induced extraembryonic-like lineages and wildtype ESCs (e.g., human ESCs) are capable of self-organization into embryo-like structures, which mimic several hallmarks of post-implantation development, including lumenogenesis, amniogenesis, primordial germ cell formation, and specification of the anterior hypoblast. These inducible embryoids are modular, do not rely on exogenous signaling factors, and are amenable to genetic perturbation. In some embodiments, the inducible embryoids generated herein are human embryoids. Some of the methods, compositions, and culture media disclosed herein are also described in “Weatherbee, B. A. T. et al. Pluripotent stem cell-derived model of the post-implantation human embryo. Nature (2023), Published online: 27 Jun. 2023, doi.org/10.1038/s41586-023-06368-y”, which is hereby incorporated by reference in its entirety.

Provided herein include methods for generating synthetic embryos in vitro from mammalian pluripotent stem cells such as pluripotent embryonic stem cells or ESCs. The method can comprise co-culturing a wild-type mammalian embryonic stem cell (ESC), a first modified mammalian ESC comprising GATA6 gene and/or SOX17 gene, and a second modified mammalian ESC comprising GATA3 gene and/or TFAP2C gene under a condition in a culture medium allowing the ESCs to self-organize into a post-implantation embryo structure. In some embodiments, the pluripotent embryonic stem cells are human pluripotent embryonic stem cells (hESCs) and the generated synthetic embryo is a human embryo. The pluripotent embryonic stem cell or ESC can be an ESC across the pluripotency spectrum including, for example, naïve ESCs, formative ESCs, or primed ESCs. For example, the ESCs used herein can be a pre-implantation naïve ESCs, peri-implantation-like pluripotent ESCs, or post-implantation primed ESCs. In some embodiments, the ESCs used herein are peri-implantation like pluripotent ESCs such as peri-implantation like pluripotent hESCs (e.g., RSeT hESCs). In some embodiments, the ESCs used herein express low levels of amnion-specific genes during trophoblast-like cell induction compared to ESCs in other pluripotency states (e.g., primed cells). The ESCs in different pluripotent states can be pre-cultured using culture media/condition identifiable to a person skilled in the art. For example, pre-implantation naïve hESCs can be generated from culturing in a PXGL medium prior to the co-culturing. Peri-implantation-like pluripotent hESCs can be generated from culturing in a RSeT medium prior to the co-culturing. Post-implantation-like primed hESCs can be generated from culturing in an mTeSR1 medium prior to the co-culturing.

A modified mammalian pluripotent stem cells, such as ESC, can comprise one or more genes encoding one or more transcription factors that can drive generation of extraembryonic cells or extraembryonic-like cells. In some embodiments, a modified mammalian ESC is an inducible mammalian ESC comprising one or more inducible genes encoding the one or more transcription factors described herein. The inducible ESC can express the one or more inducible genes upon induction. For example, a modified mammalian ESC can comprise an inducible GATA6 gene alone. Alternatively or in addition, a modified mammalian ESC can comprise an inducible SOX17 gene alone. In some embodiments, a modified mammalian ESC comprises an inducible GATA6 gene and an inducible SOX17 gene. The pluripotent stem cells comprising an inducible GATA6 gene and/or an inducible SOX17 gene can be in any pluripotency state. For example, a modified mammalian ESC comprising an inducible GATA6 gene alone can be in a pre-implantation naïve state or an intermediate peri-implantation-like state. A modified mammalian ESC comprising an inducible SOX17 gene alone can be in a pre-implantation naïve state, an intermediate peri-implantation-like state, or a post-implantation primed state. A modified mammalian ESC comprising an inducible GATA6 gene and an inducible SOX17 gene can be in a pre-implantation naïve state, an intermediate peri-implantation-like state, or a post-implantation primed state.

A modified mammalian ESC can comprise an inducible GATA3 gene alone. Alternatively or in addition, a modified mammalian ESC can comprise an inducible TFAP2C gene alone. In some embodiments, a modified mammalian ESC comprises an inducible GATA3 gene and an inducible TFAP2C gene. The pluripotent stem cells comprising an inducible GATA3 gene and/or an inducible TFAP2C gene can be in any pluripotency state. For example, a modified mammalian ESC comprising an inducible TFAP2C gene alone can be in a pre-implantation naïve pluripotent stem cells such as PXGL cell or a peri-implantation pluripotent stem cell such as RSeT cell. A modified mammalian ESC comprising an inducible GATA3 gene alone can be in a pre-implantation naïve state, an intermediate peri-implantation-like state, or a post-implantation primed state. A modified mammalian ESC comprising an inducible GATA3 gene and an inducible TFAP2C gene can be in a pre-implantation naïve state, an intermediate peri-implantation-like state, or a post-implantation primed state.

In some embodiments, GATA6 and/or SOX17 can drive the pluripotent stem cells (e.g., ESCs) to develop into hypoblast-like cells. In some embodiments, GATA3 and/or TFAP2C can drive the pluripotent stem cells (e.g., ESCs) to develop into trophoblast-like cells.

In some embodiments described herein, a modified mammalian ESC is an induced mammalian ESC expressing or overexpressing the one or more genes encoding the one or more transcription factors described herein. In some embodiments, a modified ESC (e.g., hESC) can overexpress one or more of the transcription factors described herein. For example, a modified ESC comprising GATA6 gene and/or SOX17 gene demonstrates an at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 220-fold, 240-fold, 260-fold, 270-fold, 280-fold, 300-fold or greater increase in GATA6 and/or SOX17 mRNA expression than a wild type ESC. In some embodiments, a modified ESC comprising GATA3 gene and/or TFAP2C gene demonstrates an at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 220-fold, 240-fold, 260-fold, 270-fold, 280-fold, 300-fold or greater increase in GATA3 and/or TFAP2C mRNA expression than a wild type ESC.

The method can further comprise contacting the inducible mammalian ESCs with an inducer (e.g., doxycycline) to generate induced mammalian ESCs. The induction can occur during or prior to the co-culturing of the modified ESCs with the wild-type ESCs. The contacting can be performed for any duration suitable to increase the mRNA expression of the transcription factors (e.g., GATA6, SOX17, GATA3, and/or TFAP2C) to a desired level. In some embodiments, the inducer can be provided to the culture media during the co-culturing process. For example, the inducer can be supplied or administrated to the culture medium comprising a wild type ESC and modified inducible ESCs for a duration of about, at least, at least about, at most or at most about 2 hours, 4 hours, 6 hours, 8 hours, 10 hours, 12 hours, 14 hours, 16 hours, 18 hours, 20 hours, 22 hours, 1 day, 2 days, 3 days, 4 days, 5 days, 6 days, 7 days or longer. In some embodiments, the inducer is supplied during the entire co-culturing process. Alternatively or in addition, modified mammalian ESCs can be induced prior to the co-culturing. For example, inducible ESCs can be induced (e.g., in the presence of an inducer), prior to co-culturing with the wild-type ESCs, to generate induced ESCs expressing or overexpressing the one or more transcription factors (e.g., GATA6, SOX17, GATA3 and TFAP2C). The induced ESCs are then co-cultured with the wild-type ESCs under a condition allowing the ESCs to self-organize into an aggregated structure. The duration of the induction and/or the concentration of the inducer can be modulated, for example, by increasing or decreasing the concentration or amount of the inducer in the culture medium or by increasing or decreasing of the duration time of the inducer present in the culture medium. In some embodiments, the induction can be terminated by removing the inducer from the culture medium by, for example, replacing or replenishing the culture medium with fresh culture medium free of the inducer.

The numbers or amounts of wild type ESCs and modified ESCs can be at any suitable ratio which can vary in different embodiments. In some embodiments, ESCs comprising GATA6 gene and/or SOX17 gene and/or ESCs comprising GATA3 gene and/or TFAP2C gene are provided in excess of the wild type ESCs. In some embodiments, ESCs comprising GATA3 gene and/or TFAP2C gene are provided in excess of ESCs comprising GATA6 gene and/or SOX17 gene. ESCs comprising GATA3 gene and/or TFAP2C gene can be provided at an amount or a cell count at least 2, 3, 4 5, 6, 7, or 8 times greater than the wild type ESCs and/or the ESCs comprising GATA6 gene and/or SOX17 gene. In some embodiments, ESCs comprising GATA6 gene and/or SOX17 gene and wild-type ESCs are provided in about the same amount (e.g., at a ratio of 1:1). In some embodiments, the ratio between the wild type ESCs, the ESCs comprising GATA6 gene and/or SOX17 gene, and the ESCs comprising GATA3 gene and/or TFAP2C gene is from about 1:1:2 to about 1:1:10, such as 1:1:2, 1:1:3, 1:1:4, 1:1:5, 1:1:6, 1:1:7, 1:1:8, 1:1:9, 1:1:10 or higher. In some embodiments, the ratio between the wild type ESCs, the ESCs comprising GATA6 gene and/or SOX17 gene, and the ESCs comprising GATA3 gene and/or TFAP2C gene is about 1:1:2. In some embodiments, the ratio between the wild type ESCs, the ESCs comprising GATA6 gene and/or SOX17 gene, and the ESCs comprising GATA3 gene and/or TFAP2C gene is about 1:1:1.

In some embodiments, co-culturing of wild-type ESCs, ESCs comprising GATA6 gene and/or SOX17 gene, and ESCs comprising GATA3 gene and/or TFAP2C gene is performed for a duration of about, at least, at least about, at most or at most about 1, 2, 3, 4, 5, 6 or 7 days. In some embodiments, the co-culturing comprises co-culturing wild-type ESCs, ESCs comprising GATA6 gene and/or SOX17 gene, and ESCs comprising GATA3 gene and/or TFAP2C gene in a culture medium suitable for pluripotent stem cell proliferation (e.g., a N2B27 medium). In some embodiments, culturing in a pluripotent stem cell proliferation medium is for a duration of about 5 days, optionally passaging the ESCs in the stem cell proliferation medium at least two times. During culturing in the stem cell proliferation medium, the ESCs can aggregate and form an aggregated structure exhibiting distinctions between inner and outer cellular domains, e.g., 3 days following culturing in the stem cell proliferation medium. The ESCs can be further cultured in the pluripotent stem cell proliferation medium for about 2 more days post-aggregation before transferring to a post-implantation culture medium. The method further comprises co-culturing the ESCs in a post-implantation culture medium (e.g., a post-implantation human embryo media such as hIVC1), following co-culturing in the pluripotent stem cell proliferation medium. Culturing in the post-implantation culture medium can be performed for a duration of about, at least, at least about 1 day, 2 days, 3 days, 4 days, 5 days or longer. Co-culturing the ESCs in the post-implantation culture medium can begin about 2 days post-aggregation of the ESCs. In some embodiments, co-culturing the ESCs comprises transferring the ESCs from one substrate to another substrate.

Accordingly, in some embodiments, the method comprises co-culturing a wild-type mammalian embryonic stem cell (ESC), a first modified mammalian ESC comprising GATA6 gene and/or SOX17 gene, and a second modified mammalian ESC comprising GATA3 gene and/or TFAP2C gene in a culture medium suitable for pluripotent stem cell proliferation (e.g., a N2B27 medium) under a condition allowing the ESCs to form an aggregated structure. The method can further comprise culturing the aggregated structure in a post-implantation culture medium under a condition allowing the aggregated structure to develop into a synthetic embryo mimicking a post-implantation embryo structure. Cell aggregates formed by wild-type ESCs, ESCs comprising GATA6 gene and/or SOX17 gene, and ESCs comprising GATA3 gene and/or TFAP2C gene can self-organize into a synthetic embryo structure mimicking a post-implantation stage embryo structure. The post-implantation embryo structure can be a human embryo structure. In some embodiments, the post-implantation embryo structure resembles a post-implantation human embryo at about 8-9 days post-fertilization.

The synthetic embryo structure generated using the methods and culture media described herein comprises both the embryonic and extraembryonic tissues and recapitulates embryo morphology. A post-implantation embryo structure described herein (e.g., a human post-implantation embryo structure) can comprise a pluripotent epiblast-like domain surrounded by hypoblast- and trophoblast-like tissues. In some embodiments, the post-implantation embryo structure comprises a SOX2-positive, epiblast like domain containing a central lumen; an outer single layer of GATA3-positive putative trophoblast-like cells; and an intermediate putative hypoblast-like domain of GATA6-positive cells between inner lumenized domain and outer layer.

The efficiency of forming a post-implantation embryo structure from co-culturing wild type and modified ESCs can vary in different embodiments depending on factors such as the pluripotency state of the ESCs, the expression level of the one or more transcription factor described herein, cell culture time, time and strength of the induction, and/or other factors identifiable to a person skilled in the art upon reviewing the present disclosure. For example, in some embodiments, using ESCs in an intermediate pluripotency state such as peri-implantation pluripotent ESCs (e.g., RSeT hESCs) can generate a post-implantation embryo at a greater efficiency in comparison to using ESCs at other pluripotency states (e.g., pre-implantation naïve ESCs or post-implantation primed ESCs). In some embodiments, the efficiency of forming a post-implantation embryo from co-culturing wild-type ESCs, ESCs comprising GATA6 gene and/or SOX17 gene, and ESCs comprising GATA3 gene and/or TFAP2C gene is greater than 5%, 10%, 15%, 20%, 25%, 30%, 35%, or higher. In some embodiments, the efficiency of forming a post-implantation embryo from ESCs described herein can be greater than 20% (e.g., 21%, 22%, 23%, 24%, 25%, 26%, 27%, 28%, 29%, 30% or higher).

The synthetic embryo structure described herein is an ESC-derived embryo model capable of forming a pluripotent epiblast-like domain surrounded by hypoblast- and trophoblast-like tissues. Specifically, the trophoblast and hypoblast cells are derived or programmed from ESCs (e.g., hESCs). Accordingly, the method does not comprise culturing trophoblast cells, hypoblast cells, or both. In particular, the method does not comprise culturing trophoblast cells and/or hypoblast cells in combination with pluripotent embryonic stem cells.

In some embodiments, the methods disclosed herein do not comprise any in vivo step. In some embodiments, none of the wild-type ESCs, comprising GATA6 gene and/or SOX17 gene, and ESCs comprising GATA3 gene and/or TFAP2C gene, and the synthetic embryo is present in an in vivo environment in any of the culturing steps disclosed herein. The in vivo environment can comprise a tissue, an organ, an organism, or a combination thereof.

The methods, compositions, and culture media described herein also eliminate the need for an exogenous signaling pathway that can otherwise compromise tissue-driven self-organization. In some embodiments, the methods, compositions, and culture media described herein for generating a synthetic embryo (e.g., a human synthetic embryo) does not comprise the use an exogenous signaling pathway factor (e.g., a WNT signaling pathway activator and/or a TGFβ superfamily member). For example, none of the culture media used herein comprises or is supplemented with an exogenous signaling pathway factor.

### Pluripotent Embryonic Stem Cells and Embryo Development

Disclosed herein are methods, compositions and culture media for in vitro culturing synthetic embryos from mammalian pluripotent stem cells (e.g., ESCs). In some embodiments, the mammalian pluripotent ESCs are hESCs. In some embodiments, the method comprises co-culturing a wild-type mammalian embryonic stem cell (ESC), a first modified mammalian ESC comprising GATA6 gene and/or SOX17 gene, and a second modified mammalian ESC comprising GATA3 gene and/or TFAP2C gene under a condition in a culture medium allowing the ESCs to self-organize into a synthetic embryo mimicking a post-implantation embryo structure. In some embodiments, the generated post-implantation embryo structure represents a multi-lineage stem cell-derived model of the human post-implantation embryo that undergoes lumenogenesis in its epiblast-like domain and differentiation events that reflect interactions between extraembryonic-like and embryonic-like tissues.

While mammalian embryogenesis has some common features across all species, it will be appreciated that different mammalian species develop in different ways and at different rates. In general, though, the fertilized egg undergoes a number of cleavage steps (passing through two-cell, four-cell and eight-cell stages) before undergoing compaction to form a solid ball of cells called a morula, in which the cells continue to divide. Ultimately the internal cells of the morula give rise to the inner cell mass and the outer cells to the trophectoderm. The morula in turn develops into the blastocyst, which is surrounded by trophectoderm and contains a fluid-filled vesicle, with the inner cell mass at one end.

The term “embryo” as used herein refers to a mammalian organism from the single cell stage. The embryo described herein is generated from culturing in vitro embryonic stem cells under appropriate conditions and resembles or mimics a natural embryo produced in vivo of a corresponding stage, such as having similar morphology, length, weight, cell type compositions and expression of developmental marker genes.

A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo. The earliest Carnegie stages are as follows in Table 1.

TABLE 1
Carnegie Stages of Development

| Carnegie | Days since | Characteristic events/ |
|---|---|---|
| stage | ovulation (approx.) | structures |
| 1 | 1 | fertilization; polar bodies |
| 2 | 2-3 | cleavage; morula; compaction |
| 3 | 4-5 | blastocyst and blastocoele; |
| | | trophoblast and embryoblast |
| 4 | 6 | syncytiotrophoblast; cytotrophoblast; |
| | | anchoring to endometrium |
| 5(a) | 7-8 | implantation; embryonic disc; |
| | | bilaminar germ disc; |
| | | primary yolk sac; |
| 5(b) | 9-10 | formation of trophoblast lacunae; |
| | | complete penetration into |
| | | endometrium; amniotic cavity; |
| | | primary umbilical vesicle |
| 5(c) | 11-16 | pre-chordal plate; |
| | | extra-embryonic mesoblast; |
| | | secondary yolk sac |
| 6 | 17 | primitive streak, primitive node, |
| | | primitive groove; secondary umbilical |
| | | vesicle; primordial germ cells; body |
| | | stalk; early gastrulation. |
| 7 | 19 | Gastrulation; neural plate; |
| | | start of hematopoiesis. |
| 8 | 23 | Primitive pit |
| 9 | 25 | Neural groove; neural folds; |
| | | septum transversum; placode; |
| | | early heart. |

In some embodiments, the methods, compositions, and culture media described herein can enable culture up to post-implantation stages corresponding to Carnegei stage (a), 5(b), 5(c), 6, 7, 8, 9 and beyond, and corresponding stages in other species.

The methods, compositions, and culture media herein described can be applied to embryos from any suitable mammalian species including human and non-human, such as: primates, including humans, great apes (e.g. gorillas, chimpanzees, orangutans), old world monkeys, new world monkeys; rodents (e.g. mice, rats, guinea, pigs, hamsters); cats; dogs; lagomorphs (including rabbits); cows; sheep; goats; horses; pigs; and any other livestock, agricultural, laboratory or domestic mammals. The methods, compositions, and culture media herein described can be applied to an embryo from a human. Thus, any of the culture media embodiments defined herein can support development of a human embryo in vitro on a substrate from a pre-implantation stage of development to a post-implantation stage of development.

The term “pre-implantation stage” can be used herein to refer to a stage of development earlier than the stage corresponding to Carnegie stage 5(a), and corresponding stages in other species. As used herein, the term “post-implantation stage” can refer to a stage of development later than the stage corresponding to, Carnegie stage 5(a), and corresponding stages in other species. A “post-implantation stage” may be determined by detecting the up-regulation of one or more genes by the embryo. For example, such a stage may be determined by detecting one or more of the following changes: the epiblast up-regulates Fgf5; the primitive endoderm differentiates into visceral endoderm that up-regulates Cer1 in a subpopulation of cells (the anterior visceral endoderm); the visceral endoderm up-regulates Eomes; and the trophectoderm up-regulate Handl.

Stem cells (e.g., mammalian pluripotent stem cells such as embryonic stem cells) can be cultured using the media, kits and methods described herein. In the embodiments described herein, the stem cells comprise pluripotent stem cells (PSCs). A PSC can be obtained from a fertilized egg, clone embryo, reproductive stem cell, or stem cell in tissue. Also included are cells having differentiation pluripotency similar to that of embryonic stem cells, conferred artificially by transferring several different genes to a somatic cell (also referred to as induced pluripotent stem cells or iPS cells). Induced pluripotent stem cells may be derived from any suitable source (e.g. hair follicles, skin cells, fibroblasts, etc.). Pluripotent stem cells can be prepared by known methods in the art. Any of the stem cells as defined herein may be derived from diseased or non-diseased tissue. Stem cells can be from any suitable mammalian species, such as: primates, including humans, great apes (e.g. gorillas, chimpanzees, orangutans), old world monkeys, new world monkeys; rodents (e.g. mice, rats, guinea pigs, hamsters); cats; dogs; lagomorphs (including rabbits); cows; sheep; goats; horses; pigs; and any other livestock, agricultural, laboratory or domestic mammals. The presently disclosed methods may be applied to stem cells from any non-human mammal, including but not limited to those described above. In some embodiments, the non-human mammals are rodents. In some embodiments, the PSC cells disclosed herein are mammalian embryonic stem cells (ESCs).

The pluripotent stem cells (e.g., ESCs) used herein can be in different pluripotency states. The pluripotent embryonic stem cells can be naïve ESCs, primed ESCs, or possess an intermediate state of pluripotency between the naïve and the primed pluripotent stem cells, such as formative ESCs. The pluripotent stem cells can be pre-implantation naïve stem cells, peri-implantation pluripotent stem cells, or post-implantation primed stem cells. Naïve ESCs comprise cells exhibiting naïve features, such as global DNA hypomethylation, expression of naïve pluripotency markers such as Klf4, Tfcp2l1, Esrrb, Klf2, Tbx3, Prdm14, and Dppa3, active mitochondria, reduced glucose dependence, and the capability of being propagated by enzymatic dissociation of single cells as will be understood by a person skilled in the art. Markers highly expressed in naïve ESCs can be silenced in formative and primed ESCs. Formative ESCs can possess up-regulated Otx2, Dnmt3b, Fgf5, Zic2/5, Etv1/4, Oct6, and Grhl2, which have been reported to play important roles in the transition from naïve to primed state. For example, when cells exit naïve pluripotency, increased Otx2 binds to many enhancer regions of the formative genes, which are or are not prebound by Oct4 and activates the correlated genes. Therefore, the Oct4-Otx2 regulatory axis actively establishes a new regulatory chromatin landscape to exit from naïve pluripotency and transit into a formative state. Primed pluripotent stem cells possess up-regulated Sox11, Zic23, Dusp6, and Cd24 and down-regulated or silenced naïve markers (e.g., silenced Klf4). Additional information related to pluripotent states in mammalian cells such as in mouse and human cells can be found, for example, in Genes (Basel). 2022 August; 13(8): 1459, doi: 10.3390/genes13081459, the content of which is incorporated by reference in its entirety.

In some embodiments, the pluripotent stem cells used herein for generating a synthetic embryo are naïve cells (e.g., naïve ESCs or naïve hESCs). In some embodiments, the pluripotent stem cells used herein are intermediate stem cells of formative pluripotency around implantation or at early implantation stage. In some embodiments, the pluripotent stem cells used herein can readily differentiate to peri- and post-implantation yok sac-like endoderm cells compared to naïve cells such as PXGL cells. In some embodiments, the ESCs used herein can be pre-implantation naïve hESCs, peri-implantation-like pluripotent hESCs, or post-implantation primed hESCs. In some embodiments, the ESCs are peri-implantation pluripotent hESCs. The peri-implantation pluripotent hESCs can exhibit a naïve-like state such as tightly packed, domed colonies with refractive edges and show increased expression of gene markers such as Klf2, Klf4 and Tfcp2L1. The ESCs used herein can express low levels of amnion-specific genes during trophoblast-like cell induction compared to ESCs at other pluripotency states (e.g., primed cells).

Pluripotent stem cells (e.g., ESCs) in different pluripotent states can be pre-cultured using suitable culture media/conditions identifiable to a person skilled in the art. For example, the pre-implantation naïve hESCs can be generated from culturing in a PXGL medium prior to the co-culturing. The peri-implantation-like pluripotent hESCs can be generated from culturing in a RSeT medium prior to the co-culturing. The post-implantation-like primed hESCs can be generated from culturing in an mTeSR1 medium prior to the co-culturing.

The conversion among diverse pluripotent states can be achieved in vitro. In some embodiments, primed pluripotent stem cells (e.g., hESCs) can be converted to formative or naïve cells under a suitable culture condition as will be understood by a person skilled in the art. For example, to convert primed pluripotent stem cells (e.g., primed hESCs) to peri-implantation pluripotent stem cells (e.g., RSeT cells), primed hESCs can be passaged to mitomycin-C inactivated CF-1 MEFs in media comprising DMEM/F12 with Knockout Serum Replacement, a reducing agent, non-essential amino acids, antibiotics, L-glutamine or an analogue thereof, a fibroblast growth factor family (FGF) member, and a ROCK inhibitor. To culture peri-implantation pluripotent stem cells (e.g., RSeT cells), the media can be switched to a medium suitable for culturing naïve-like pluripotent stem cells such as a RSeT medium. RSeT medium is a defined cell culture medium used for the reversion of primed human pluripotent stem cells (hPSCs) to a naïve-like state and maintenance of naïve-like hPSCs under feeder-dependent and hypoxic conditions. In some embodiments, RSeT medium does not contain bFGF or TGFβ. RSeT medium is compatible with human embryonic stem cells and human induced pluripotent stem cells. Additional information related to culture condition for naïve cells (e.g., PXGL cells) can be found in the Example section as well as in “Bredenkamp, N. et al., Wnt Inhibition Facilitates RNA-Mediated Reprogramming of Human Somatic Cells to Naive Pluripotency. Stem cell reports 13, 1083-1098 (2019). //doi.org:10.1016/j.stemcr.2019.10.009”, the content of which is hereby incorporated by reference in its entirety.

As would be understood by a person of skill in the art, pluripotent stem cells such as ESCs may be obtained from stem cell banks such as the UK stem cell bank from which one can acquire human stem cell lines for research or obtained from participating fertility facilitates. It is preferred that the ESCs are obtained or are obtainable by a method that does not involve the destruction of human or non-human animal embryos.

### Synthetic Embryo Structures

Provided herein include methods, compositions, and culture media for modeling mammalian embryo development by culturing pluripotent stem cells (e.g., embryonic stem cells). The methods, compositions and culture media disclosed herein can generate synthetic embryo structure through co-culturing wild type embryonic stem cells and extraembryonic-like cells generated by transcription factor overexpression with wild type embryonic stem cells. Accordingly, provided herein also includes a mammalian embryo structure comprising embryonic and extraembryonic tissues. In some embodiments, the mammalian embryo structure generated using the methods, compositions and culture media described herein is a post-implantation embryo model structure such as a human embryoid. In some embodiments, the synthetic embryo structure represents self-organized aggregates containing a pluripotent epiblast-like domain surrounded by extraembryonic-like tissues, in which the epiblast-like domain can differentiate to amnion, extraembryonic mesenchyme, and primordial germ cell-like cells in response to BMP signaling.

In some embodiments, the wild type and modified pluripotent stem cells used herein can form cell aggregates within 24 hours of culturing in a culture medium (e.g., a stem-cell proliferation medium). About four days post-aggregation, the cell aggregates can self-organize into structures with a SOX2-positive, epiblast-like domain containing a central lumen, an outer single layer of GATA3-positive putative trophoblast-like cells, and an intermediate putative hypoblast-like domain of GATA6-positive cells between inner lumenized domain and outer layer. The synthetic embryo structure can comprise aggregates containing an organized SOX2-positive domain surrounded by concentric layers of GATA6-positive and GATA3-positive cells. In some embodiments, the aggregates do not transit through a blastocyst-like morphology prior to forming post-implantation-like structures.

In some embodiments, the synthetic embryo structures generated using the methods and compositions described herein can reach a post-implantation stage (e.g., a post-implantation gastrulating stage). In some embodiments, the synthetic embryo structure generated herein can reach an early gastrulation stage. In some embodiments, the synthetic embryos generated herein can reach a late gastrulation stage. As used herein, the term “gastrulation” in the context of an embryo refers to an embryo following the expanded blastocyst stage and prior to the somitogenesis stage and is characterized by the formation of the primitive streak and epithelial to mesenchymal transition forming three germinal layers. The gastrulation process is generally considered as the process through which the bilaminar embryonic disc is changed into a trilaminar disc as an intraembryonic mesoderm appears between the ectoderm and endoderm. A gastrulation stage can be an early gastrulation stage, mid-gastrulation stage, or late or advanced gastrulation stage.

In some embodiments, the embryo structures generated using the methods and culture media described herein comprise post-implantation embryos, e.g., post-implantation pre-gastrulation embryo structure. As used herein, the term “post implantation pre gastrulation” in the context of a mammalian embryo (e.g., a human embryo) refers to an embryo following the implanting blastocyst stage and prior to the early gastrulation stage and is characterized by an egg cylinder-shape prior to symmetry breaking.

Embryonic stage of a synthetic embryo structure generated using the methods and culture media disclosed herein can be assessed by comparing to an in vivo natural embryo counterpart at the same developmental stage by multiple ways including, but not limited to, morphology, length, weight, cell type compositions, chromatin accessibility patterns, expression of developmental marker genes (e.g., Oct4, Nanog, Sox2, Klf4, Cdx2, Gata4, Gata6, Brachyury, Otx2, Fgf5 and others described in the Examples and known in the art) using specific antibodies or primers, or transcriptional profiling, single-cell RNA sequencing and other methods as further described in the Examples section.

In some embodiments, the post-implantation embryo structure generated herein comprises an inner epiblast-like domain, a single outer layer of trophoblast-like cells, and an intermediate hypoblast-like domain between the epiblast-like domain and the single outer layer of trophoblast-like cells. The inner epiblast-like domain can be SOX2 positive and contains a central lumen, the single outer layer of trophoblast-like cells can be GATA3 positive, and the intermediate hypoblast-like domain can be GATA6 positive. In some embodiments, the post-implantation embryo structure expresses N-Cadherin and SOX17 in the hypoblast-like domain, CDX2 in the trophoblast-like cells, and/or SOX2, NANOG and E-Cadherin in the epiblast-like domain. The inner epiblast-like domain can exhibit a pluripotent and epithelial identity akin to a human embryo.

In some embodiments, the post-implantation embryo structure generated herein comprises cell clusters resembling embryonic late-epiblast, amnion, mesoderm, extraembryonic mesenchyme, and/or hypoblast/visceral endoderm. In some embodiments, the post-implantation embryo structure expresses TDGF1, SOX2, NANOG, TFAP2A, ID1, ISL1, TFAP2C, VTCN1, GRHL1, MEIS1, TBXT, MESP1, MIXL1, CER1, SNAI1, EOMES, POSTN, COL6A3, IGF2, TBX20, BMP6, CDH2, HNF1B, FOXA2, VTCN1, HAND1, TBX20, CDX2, PRDM1, OCT4, or a combination thereof.

In some embodiments, the post-implantation embryo structure can generate amnion and primordial germ cells. The post-implantation embryo structure can generate primordial germ cell-like cells expressing the pluripotency marker NANOG and primordial germ cell markers PRDM1 (BLMP1) and NANOS3.

BMP signaling can play a role during differentiation of epiblast-like domain. In some embodiments, the human embryo structure generated herein can express one or more of downstream BMP response genes of ID1, ID2, ID3 or ID4 (see, for example, Example 4). In some embodiments, phosphorylated (p)SMAD1.5 expression in the OCT4-positive epiblast-like domain at days 4 and 6 post-aggregation is indicative of active BMP signaling.

One or more of the gene markers described herein can be upregulated or downregulated in the generated synthetic embryo structure by one or more of the inducible genes introduced to the pluripotent stem cells. For example, induction of SOX17 alone or in combination with GATA6 can result in decreased capacity to upregulate CER1, as compared with GATA6 overexpression alone (see, for example, Example 5). Alternatively or in addition, synthetic embryo structure generated with single GATA6 induction or reduced induction strength and/or duration (e.g., doxycycline withdrawal after a certain time period, e.g., at day 3) show increased expression primitive streak marker BRY/TBXT at day 6 post-aggregation as compared to structures with consistent GATA6-SOX17 or SOX17 induction. Accordingly, in some embodiments, the in vitro synthetic embryo structure generated herein can be used as a modular embryoid model to study gene marker regulation and interactions between embryonic and extraembryonic tissues, to interrogate the role of specific tissues and tissue-specific gene requirements, and to investigate mechanisms involved in embryogenesis.

In some embodiments, the synthetic embryo structures generated using the methods and culture conditions described herein are mammalian embryo structures. In some embodiments, the mammalian embryo structures are human embryo structures, such as a human embryoid. The human embryo-like structure generated herein can exhibit an organization reminiscent of a human embryo at about 8-9 days post-fertilization.

Culturing an embryo cell in vitro from stem cells (e.g., pluripotent ESCs) can be effected until reaching post-implantation or any developmental stage therein-between. In some embodiments, the synthetic embryo generated using the methods and culture media described herein do not mimic stages beyond primitive streak formation. In some embodiments, the synthetic embryo generated using the methods and culture media described herein may not contain all cell types of a gastrulation-stage embryo. In some embodiments, the synthetic embryo generated using the methods and culture media described herein may not further develop to form viable human embryos.

Culturing conditions mentioned above for generating synthetic embryos, including substrates, culture media, and the like, are described in the sections below as well as in specific embodiments of human embryos in the Examples section.

Embryonic stages of the synthetic embryos described herein can be assessed compared to an in vivo or natural embryo counterpart at the same developmental stage by multiple ways including, but not limited to, morphology, length, weight, weight, expression of developmental marker genes using specific antibodies or primers, transcriptional profiling and the like, as further described hereinbelow and in the Examples section.

Morphology assessment of embryonic development can be performed by previously established morphological features such as described in Carnegie stages of development (see, for example, Table 1; Developmental stages in human embryos. R. O'Rahilly and F. Müller (eds), Carnegie Institution of Washington, Washington, D C, 1987) or according to embryonic days.

In some embodiments, one or more developmental markers as described herein can be used to assess the developmental stage of a synthetic embryo structure. Numerous methods exist in the art for detecting the presence, absence, or amount of a marker gene product (e.g., mRNA and/or protein), as well as its localization in an embryo structure or subcellular localization (e.g., nucleus and/or cytoplasm). Marker expression may be assessed by any of a wide variety of well-known methods for detecting expression of a transcribed molecule or a protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification and sequencing methods.

In some embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a marker and functionally similar homologs thereof, including a fragment or genetic alteration thereof (e.g., in regulatory or promoter regions thereof) comprises detecting or determining RNA levels for the marker of interest. In some embodiments, one or more cells from the synthetic embryo structure can be obtained and RNA is isolated from the cells. In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated. It is also be possible to obtain cells from, e.g., the synthetic embryo cells and culture the cells in vitro, such as to obtain a larger population of cells from which RNA can be extracted. Methods for establishing cultures of non-transformed cells, i.e., primary cell cultures, are known in the art. In some embodiments, cells can be dissociated (e.g., by enzymatic or mechanical means), and isolated by methods known in the art (e.g., Fluorescence-Activated Cell Sorting, Microfluidics, etc.)

When isolating RNA from, e.g., synthetic embryo structures at various developmental stages and/or cells comprising said synthetic embryo structures, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from cells by a variety of methods, e.g., the guanidium thiocyanate lysis followed by CsCl centrifugation. Methods for obtaining RNA from single-cells are also known in the art. The RNA sample can then be enriched in particular species. In some embodiments, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.). In some embodiments, the RNA population is enriched in marker sequences. Enrichment can be undertaken, e.g., by primer-specific cDNA synthesis, or multiple rounds of linear amplification based on cDNA synthesis and template-directed in vitro transcription.

The population of RNA, enriched or not in particular species or sequences, can further be amplified. As defined herein, an “amplification process” increases the number of copies of a polynucleotide (e.g., RNA). For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the disclosed methods to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used. Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)). Many techniques are known in the state of the art for determining absolute and relative levels of gene expression, commonly used techniques suitable for use in the disclosed methods include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used. In some embodiments, the probe is labeled with a fluorescence moiety.

Alternatively, mRNA expression can be detected on a DNA array, chip or a microarray. Labeled nucleic acids of a test sample obtained from a subject may be hybridized to a solid surface comprising marker DNA. Positive hybridization signal is obtained with the sample containing marker transcripts. Methods of preparing DNA arrays and their use are well known in the art (see, e.g., U.S. Pat. Nos. 6,186,796; 6,379,897; 6,664,377; 6,451,536; 548,257; U.S. 20030157485). Serial Analysis of Gene Expression (SAGE) can also be performed (See for example U.S. Patent Application 20030215858). In some embodiments, next generation sequencing (e.g., RNA-seq) can be used to analyze total mRNA expression from one (e.g., single-cell RNA-seq) or more cells. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. Methods for constructing sequencing libraries are known in the art.

In some aspects of the disclosure the single cell sequencing is high-throughput single cell RNA sequencing. In certain embodiments, the single cell sequencing is a low cost high-throughput single cell RNA sequencing. Not being bound by any particular theory, the single cell RNA sequencing is capable of efficiently and cost effectively sequencing thousands to tens of thousands of single cells. In certain embodiments, single cell RNA sequencing comprises pairing single cells in droplets with oligonucleotides for reverse transcription, wherein the oligonucleotides are configured to provide cell-of-origin specific barcodes uniquely identifying transcripts from each cell and a unique molecular identifier (UMI) uniquely identifying each transcript. In certain embodiments, single cell RNA sequencing comprises pairing single cells in droplets with single microparticle beads coated with oligonucleotides for reverse transcription, wherein the oligonucleotides contain a bead-specific barcode uniquely identifying each bead and a unique molecular identifier (UMI) uniquely identifying each primer. In some aspects of the disclosure, unbiased classifying of cells in a biological sample comprises sequencing the transcriptomes of thousands of cells, preferably tens of thousands of cells (e.g., greater than 1000 cells, or greater than 10,000 cells).

The activity or level of a lineage marker protein can be detected and/or quantified by detecting or quantifying the expressed polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

Described below are non-limiting examples of techniques that may be used to detect marker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-marker protein antibodies (unlabeled) are then brought into contact with the support and assayed by a secondary immunological reagent, such as labeled protein A or anti-immunoglobulin (suitable labels including ¹²⁵I, horseradish peroxidase, alkaline phosphatase, fluorophore). Chromatographic detection may also be used.

Immunohistochemistry may be used to detect expression of marker protein. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-marker protein antibodies, such as intrabodies, may also be used for imaging purposes, for example, to detect the presence of marker protein in cells or, e.g., an embryo. Suitable labels include radioisotopes, iodine (¹²⁵ I, ²¹I), carbon (¹⁴C), sulphur (³⁵S), tritium (³H), indium (¹¹²In), and technetium (⁹⁹mTc), fluorescent labels, such as fluorescein and rhodamine, and biotin.

Antibodies that may be used to detect marker protein include any antibody, whether natural or synthetic, full length or a fragment thereof, monoclonal or polyclonal, that binds sufficiently strongly and specifically to the marker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the marker protein relative to other proteins, such as related proteins.

Antibodies are commercially available or may be prepared according to methods known in the art. Antibodies and derivatives thereof that may be used encompass polyclonal or monoclonal antibodies, chimeric, human, humanized, primatized (CDR-grafted), veneered or single-chain antibodies as well as functional fragments, i.e., marker protein binding fragments, of antibodies. For example, antibody fragments capable of binding to a marker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain. In some embodiments, agents that specifically bind to a marker protein other than antibodies are used, such as peptides. Peptides that specifically bind to a marker protein can be identified by any means known in the art. For example, specific peptide binders of a marker protein can be screened for using peptide phage display libraries.

### Substrates for Culturing Synthetic Embryos

In some embodiments, wild type and modified pluripotent stem cells (e.g., ESCs), cell aggregates, post-implantation embryos, and/or synthetic embryos described herein are cultured in a substrate. In some embodiments, the method comprises transferring the ESCs and/or embryos from one substrate to another substrate. The substrates used in the methods disclosed herein can be the same or different. For example, the mammalian ESCs (e.g., WT and modified ESCs) can be cultured in a first substrate to form an aggregate structure. The cell aggregate can be transferred to a second substrate to develop into a post-implantation embryo. The first substrate and second substrate can be a same type or different types. In some embodiments, the first substrate and second substrate are of different types. In some embodiments, the first substrate and the second substrate can be a microwell plate comprising inverted pyramidal microwells, such as AggreWell™ microplates.

The substrate as used herein can comprise a dish, a U-plate, a flask, or a microwell plate. The microwell plate can comprise inverted pyramidal microwells. The size (e.g., depth and/or diameter) of each of the inverted microwells can vary. Each of the inverted-pyramidal microwells can be about 400 μm or about 800 μm in size. Each of the inverted-pyramidal microwells can be about 400 μm or about 800 μm in diameter. In some embodiments, each of the inverted pyramidal microwells can be about 100, 200, 300, 400, 500, 600, 700, 800, 900 μm, 1 mM in size and/or diameter, or a number or a range between any two of these values. Each microwell (e.g., receptacle) may have a depth of about 250 μm to about 400 μm, e.g., about 300 μm to about 350 μm. Additionally or alternatively, said plurality of receptacles may have a mean depth of about 250 μm to about 400 μm, e.g. about 300 μm to about 350 μm. Especially when the receptacles are wells, they may be ordered on the substrate in an array, i.e., in a grid pattern having regular spacing in substantially orthogonal directions. Whatever the topography of the substrate, the substrate may carry one or more embryos. Where the substrate comprises one or more receptacles, each said receptacle may independently contain one or more embryos, e.g., 2, 3, 4, 5, 6, 7, or 8 embryos, or more. In some embodiments, each embryo structure is located in a different respective well. In alternative embodiments, each receptacle comprises a plurality of embryos, e.g., 2, 3, 4, 5, 6, 7, or 8 embryos, or more.

The methods disclosed herein may be applied in culture volumes of any appropriate size. For example, the culture volume per embryo may be about 50 μl to about 10 ml, optionally about 100 μl to about 5 ml, optionally about 250 μl to about 5 ml, optionally about 1 ml to about 5 ml. The culture volume per embryo may be about 100, 150, 200, 250, 300, 350, 400, 450, 500, 550, 600, 650, 700, 750, 800, 850, 900, 950, 1000, 1500, 2000 μl or more.

### Culture Media

In some embodiments, the method comprises co-culturing a wild-type mammalian embryonic stem cell (ESC), a first modified mammalian ESC comprising GATA6 gene and/or SOX17 gene, and a second modified mammalian ESC comprising GATA3 gene and/or TFAP2C gene in a culture medium suitable for pluripotent stem cell proliferation (e.g., a N2B27 medium) under a condition allowing the ESCs to form an aggregated structure. The method can further comprise culturing the aggregated structure in a post-implantation culture medium under a condition allowing the aggregated structure to self-organize into a synthetic embryo structure mimicking a post-implantation embryo structure.

In some embodiments, the method comprises co-culturing mammalian pluripotent stem cells (e.g., ESCs) in a stem cell proliferation medium, optionally passaging the ESCs in the stem cell proliferation medium at least two times (e.g., 2, 3, 4 or more times). The mammalian pluripotent stem cells can be cultured in the stem cell proliferation medium for 1, 2, 3, 4, or 5 days. In some embodiments, the pluripotent stem cells aggregate following about 3 days of co-culturing in the stem-cell proliferation medium.

The method can also comprise co-culturing the pluripotent stem cells in a post-implantation culture medium for at least 2 days (e.g., 2, 3, 4, 5, 6 or more days), following co-culturing in the stem cell proliferation medium. In some embodiments, the pluripotent stem cells are cultured in the post-implantation culture medium for at least 2 days following culturing in the stem cell proliferation medium for about 5 days.

In some embodiments, the method comprises partially replacing a quantity of a stem cell proliferation medium (e.g., at least half of the media) with a refresh stem cell proliferation medium or a post-implantation culture medium. The replacement can occur every 20-28 hours of the culturing (e.g., every 24 hours). In some embodiments, the method comprises partially replacing a quantity of a post-implantation culture medium (e.g., at least half of the media) with a refresh post-implantation culture medium.

The culture media disclosed herein, including the stem cell proliferation medium and the post-implantation culture medium, can comprise a basal culture medium. The basal medium can comprise water, salts, amino acids, a carbon source, vitamins, lipids and a buffer. Suitable carbon sources may be assessed by one of skill in the art from compounds such as glucose, sucrose, sorbitol, galactose, mannose, fructose, mannitol, maltodextrin, trehalose dihydrate, and cyclodextrin. Basal media are commercially available, for example, under the trade names Advanced DMEM/F12 (Gibco, 12634-010) and CMRL-1066 (Invitrogen or Sigma). The basal culture medium can comprise Dulbecco's Modified Eagle Medium (DMEM), DMEM Nutrient Mixture 12 (DMEM/F12), Roswell Park Memorial Institute (RPMI) medium 1640, Neurobasal®, Neurobasal® A, Connaught Medical Research Laboratory 1066 (CMRL-1066), or any combination thereof.

The basal culture medium can comprise Dulbecco's Modified Eagle Medium (DMEM), DMEM Nutrient Mixture 12 (DMEM/F12), a non-human serum or serum substitute thereof, an antibiotic, L-glutamine or an analogue thereof (e.g., GlutaMAX™), or any combination thereof.

The non-human serum or serum substitute can comprise fetal bovine serum, bovine serum albumin, rat serum, KnockOut™ Serum Replacement, or any combination thereof. The antibiotic can comprise Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof.

The concentration or amount of one or more of the components in a solution or media can vary. The amount of, e.g., the non-human serum or serum substitute thereof, antibiotic, a reducing agent, and/or L-glutamine (e.g., GlutaMax™) can vary, and, in some embodiments, can be adjusted as needed by one of skill in the art. In some embodiments, the amount of non-human serum or serum substitute thereof can comprise about 0.01% to about 40% (e.g., about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the amount of antibiotic can comprise about 0.01% to about 10% (e.g., about 0.01%, 0.1%, 1%, 2%, 3%, 4%, 5%, 6%, 7%, 8%, 9%, 10% or a number or a range between any two of these values) volume per volume (% v/v), weight per volume (% w/v) or weight per weight (% w/w) of the medium. The amount of e.g., the reducing agent can vary. For example, in some embodiments, the concentration of the reducing agent in the composition can be about 0.1 μM to about 1 mM (e.g., about 0.1, 0.2, 0.3, 0.4, 0.5, 0.6, 0.7, 0.8, 0.9, 1, 10, 100, 200, 300, 400, 500, 600, 700, 800, 900 RM, 1 mM, or a number or a range between any two of these values). The amount of L-glutamine (e.g., GlutaMAX™) can vary. For example, in some embodiments, the concentration of L-glutamine in the culture media can be about 0.1 mM to about 40 mM, about 0.2 mM to about 20 mM, about 0.5 mM to about 10 mM, about 1 mM to about 5 mM or about 1.5 mM to about 2.5 mM e.g., about 2 mM. Where percentages are provided for agents, ingredients and compounds, they can be % w/w, % w/v or % v/v with respect to the formulation as a whole, unless otherwise indicated.

Each component of the culture medium described herein may be present in an amount such that the culture medium is suitable for supporting the self-organization of stem cells (e.g., ESCs) into a post-implantation embryo structure and/or further development of the post-implantation embryo structure.

In the embodiments described herein, the culture media and compositions used herein do not contain or are not supplemented with an exogenous signaling pathway factors. In some embodiments, the culture media and compositions used herein do not comprise a WNT signaling pathway activator (e.g., a WNT agonist or WNT signaling agonist). Exemplary WNT signaling pathway agonists include, without limitation, CHIR99021, derivatives of CHIR99021, e.g., a salt of CHIR99021, e.g., trihydrochloride, a hydrochloride salt of CHIR99021, Wnt3a recombinant protein, a glycogen synthase kinase 3 (GSK3) inhibitor, such as 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC 693868, SB 216763, SB 415286, TC-G 24, TCS 2002, TCS 21311, TWS 119, and analogs or derivatives of any of these.

In some embodiments, the culture media or compositions used herein do not comprise a TGFβ superfamily member. The “TGF-β superfamily” means proteins having structural and functional characteristics of known TGFβ family members. The TGFβ family of proteins is well characterized, both from structural and functional aspects. It includes the TGFβ series of proteins, the Inhibins (including Inhibin A and Inhibin B), the Activins (including Activin A, Activin B, and Activin AB), MIS (Müllerian inhibiting substance), BMP (bone morphogenetic proteins), dpp (decapentaplegic), Vg-1, MNSF (monoclonal nonspecific suppressor factor), and others. Activity of this family of proteins is based on specific binding to certain receptors on various cell types. Members of this family share regions of sequence identity, particularly at the C-terminus, that correlate to their function. The TGFβ family includes more than one hundred distinct proteins, all sharing at least one region of amino acid sequence identity. The TGF-β superfamily member (e.g., BMP4) can be natural or recombinant. Exemplary TGFβ superfamily members include, without limitation, growth differentiation factor 8 (GDF8) (GenBank Accession EAX10880), growth differentiation factor 11 (GDF11) (GenBank Accession AAF21630), Activin A, Nodal, Activin A, Activin B, bone morphogenic protein-2 (BMP2), bone morphogenic protein-4 (BMP4), and functional fragments of any thereof.

The mammalian pluripotent stem cells described herein, e.g., the wild type ESCs and modified ESCs with transcription factor overexpression, can be individually cultured, prior to the co-culturing described herein, in a suitable culture media suitable for stem cell and pluripotent stem cell proliferation as will be understood by a person skilled in the art. For example, the ESCs can be cultured in a culture medium free of serum or substantially free of serum or essentially free of serum. The culture medium may comprise a serum replacement medium. Such serum replacement media are commercially available under the trade names KSR (KnockOut™ Serum Replacement, Invitrogen, 10828-010) and N2B27 (e.g., Invitrogen, ME100137L1). The serum replacement medium may be included in the culture medium at about 5% to about 60%, about 10% to about 50%, about 15% to about 45%, or about 20% to about 40%. Exemplary culture media include, but are not limited to, RSeT media, PXGL media, cRM-1 media, mTeSR1 media and others identifiable to a person skilled in the art.

In some embodiments, the culture media may be supplemented with an inhibitor of rho-associated protein kinase (ROCK) (also referred to herein as ROCK inhibitor). Exemplary ROCK inhibitors include, but are not limited to N-[(1S)-2-Hydroxy-1-phenylethyl]-N′-[4-(4-pyridinyl)phenyl]-urea (AS1892802), fasudil hydrochloride (also known as HA 1077), -[3-[[2-(4-Amino-1,2,5-oxadiazol-3-yl)-1-ethyl-1H-imidazo[4,5-c]pyridin-6-yl]oxy]phenyl]-4-[2-(4-morpholinyl)ethoxy]benzamide (GSK269962), 4-[4-(Trifluoromethyl)phenyl]-N-(6-Fluoro-1H-indazol-5-yl)-2-methyl-6-oxo-1,4,5,6-tetrahydro-3-pyridinecarboxamide (GSK 429286), (S)-(+)-2-Methyl-1-[(4-methyl-5-isoquinolinyl)sulfonyl]-hexahydro-1H-1,4-diazepine dihydrochloride (H 1152 dihydrochloride), (S)-(+)-4-Glycyl-2-methyl-1-[(4-methyl-5-isoquinolinyl)sulfonyl]-hexahydro-1H-1,4-diazepine dihydrochloride (glycyl-H 1152 dihydrochloride), N-[(3-Hydroxyphenyl)methyl]-N′-[4-(4-pyridinyl)-2-thiazolyl]urea dihydrochloride (RKI 1447 dihydrochloride), (3S)-1-[[2-(4-Amino-1,2,5-oxadiazol-3-yl)-1-ethyl-1H-imidazo[4,5-c]pyridin-7-yl]carbonyl]-3-pyrrolidinamine dihydrochloride (SB772077B dihydrochloride), N-[2-[2-(Dimethylamino)ethoxy]-4-(1H-pyrazol-4-yl)phenyl-2,3-dihydro-1,4-benzodioxin-2-carboxamide dihydrochloride (SR 3677 dihydrochloride), and trans-4-[(R)-1-Aminoethyl]-N-4-pyridinylcyclohexanecarboxamide dihydrochloride (Y-27632 dihydrochloride), N-Benzyl-[2-(pyrimidin-4-yl)amino]thiazole-4-carboxamide (Thiazovivin), Rock Inhibitor, a isoquinolinesulfonamide compound (Rho Kinase Inhibitor), N-(4-Pyridyl)-N′-(2,4,6-trichlorophenyl)urea (Rho Kinase Inhibitor II), 3-(4-Pyridyl)-1H-indole (Rho Kinase Inhibitor III, Rockout), and 4-pyrazoleboronic acid pinacol ester; a Rock antibody commercially available from Santa Cruz Biotechnology selected from the group consisting of Rock-1 (B 1), Rock-1 (C-19), Rock-1 (H-11), Rock-1 (G-6), Rock-1 (H-85), Rock-1 (K-18), Rock-2 (C-20), Rock-2 (D-2), Rock-2 (D-11), Rock-2 (N-19), Rock-2 (H-85), Rock-2 (30-J); a ROCK CRISPR/Cas9 knockout plasmid selected from the group consisting of Rock-1 CRISPR/Cas9 KO plasmid (h), Rock-2 CRISPR/Cas9 KO plasmid (h), Rock-1 CRISPR/Cas9 KO plasmid (m), Rock-2 CRISPR/Cas9 KO plasmid (m); a ROCK siRNA, shRNA plasmid and/or shRNA lentiviral particle gene silencer selected from the group consisting of Rock-1 siRNA (h): sc-29473, Rock-1 siRNA (m): sc-36432, Rock-1 siRNA (r): sc-72179, Rock-2 siRNA (h): sc-29474, Rock-2 siRNA (m): sc-36433, Rock-2 siRNA (r): sc-108088. In some embodiments, the ROCK inhibitor comprises Y-27632. The ROCK inhibitor can be provided at an effective amount at a concentration of about 0.1 μM to about 100 RM. In some embodiments, a culture medium comprises a ROCK inhibitor at a concentration of about 10 RM. In some embodiments, the culture medium does not comprise a ROCK inhibitor.

The culture medium described herein may contain other components, or analogues thereof. As used herein, the term “analogue” can refer to a biologically active analogue of any of the components of the culture medium. Such an analogue may be natural or synthetic.

The specific biologically active ligands and compounds used in the media defined herein, such as insulin, progesterone, etc. are used for illustrative purposes. However, one of skill in the art will readily recognize that analogues of such ligands and compounds may equally be used as alternatives, provided that they retain the relevant biological activity. One of skill in the art will be able to identify, in a routine manner. other biologically active compounds that are suitable for use as substitutes. For instance, these may be naturally occurring compounds or compounds which can be made by synthetic or semi-synthetic methods.

Stem Cell Proliferation Medium

In some embodiments, a pluripotent stem cell proliferation medium used herein is serum-free, or substantially serum free. Alternatively, the stem cell proliferation medium may be supplemented with KSR, optionally about 5%-15% KSR. In some embodiments, the stem cell proliferation medium is a defined in vitro culture medium that is free or substantially free of serum comprising a basal medium comprising water, salts, amino acids, a carbon source, vitamins, lipids and a buffer. In some embodiments, the stem cell proliferation medium can further comprise sodium pyruvate. Sodium pyruvate may be included in the culture medium at a concentration of about 0.05 mM to about 10 mM, about 0.1 mM to about 2 mM, about 0.2 mM about 1 mM. In some embodiments, the stem cell proliferation medium comprises a neurobasal medium (e.g., Neurobasal or Neurobasal A from Thermo Fisher Scientific). The stem cell proliferation medium can further comprise or be supplemented with B-27 supplement and N-2 supplement. As will be understood by a skilled person, B-27 supplement is a defined mixture of antioxidant enzymes, proteins, vitamins, and fatty acids that are combined in optimized ratios to support neuronal survival in culture. N2-supplement is a chemically defined, serum-free supplement that can be used for growth and expression of neuroblastomas as well as post-mitotic neurons in primary cultures from both the peripheral nervous system and the central nervous system.

The stem cell proliferation medium can comprise an effective amount of L-glutamine or an analogue thereof. L-glutamine may be included in the culture medium at a concentration of about 0.1 mM to about 40 mM, about 0.2 mM to about 20 mM, about 0.5 mM to about 10 mM, about 1 mM to about 5 mM or about 1.5 mM to about 2.5 mM e.g., about 2 mM. In some embodiments, L-glutamine is included in the stem cell proliferation medium at a concentration of about 2 mM.

The stem cell proliferation medium can further comprise or be supplemented with an effective amount of a reducing agent. The reducing agent can comprise beta-mercaptoethanol (BME), N-acetyl-L-cysteine, dithiothreitol (DTT), or any combination thereof. In some embodiments, the concentration of the reducing agent in the stem cell proliferation medium can be about 0.1 μM to about 1 mM (e.g., about 0.1, 0.2, 0.3, 0.4, 0.5, 0.6, 0.7, 0.8, 0.9, 1, 10, 100, 200, 300, 400, 500, 600, 700, 800, 900 RM, 1 mM, or a number or a range between any two of these values). In some embodiments, the reducing agent is included in the stem cell proliferation medium at a concentration of about 0.1 mM. In some embodiments, the stem cell proliferation medium comprises β-mercaptoethanol (BME) at a concentration of about 0.1 mM.

In some embodiments, a stem cell proliferation medium comprises Dulbecco's Modified Eagle Media (DMEM), DMEM Nutrient Mixture 12 (DMEM/F12), Neurobasal® A, N2, B27, L-glutamine or an analogue thereof, a reducing agent, an antibiotic, or a combination thereof, wherein the components are provided in amounts such that the medium is capable of supporting the proliferation of the pluripotent stem cells on a substrate. In some embodiments, a stem cell proliferation medium comprises DMEM/F12, Neurobasal® A, B-27, N-2, GlutaMax™, 3-mercaptoethanol, penicillin/streptomycin or a combination thereof. In some embodiments, the stem cell proliferation medium is N2B27 medium. The N2B27 medium can comprise 1:1 DMEM/F12 and Neurobasal A, 0.5×B-27, 0.5×N-2, 100 μM β-mercaptoethanol, 1× GlutaMAX, and 1× penicillin-streptomycin.

Post-Implantation Culture Medium

The methods described herein also comprise co-culturing the pluripotent stem cells (e.g., cell aggregates formed by ESCs) in a post-implantation culture medium, following co-culturing in the stem cell proliferation medium. In some embodiments, the post-implantation culture medium is a post-implantation human embryo culture media (e.g., hIVC1).

A post-implantation medium can comprise a non-human serum. The non-human serum in the post-implantation culture medium can vary. In some embodiments, the post-implantation medium can comprise a non-human serum (e.g., fetal bovine serum) at about 5% to about 40% (e.g., 5%, 10%, 11%, 12%, 13%, 14%, 15%, 16%, 17%, 18%, 19%, 20%, 21%, 22%, 23%, 24%, 25%, 26%, 27%, 28%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the post-implantation medium comprises about 15% to about 30% non-human serum (e.g., fetal bovine serum). In some embodiments, the post-implantation medium comprises about 20% fetal bovine serum. In some embodiments, the fetal bovine serum is inactivated.

A post-implantation medium can further comprise (a) insulin, an insulin analogue, or an insulin receptor agonist; (b) estrogen, an estrogen analogue, or an estrogen receptor agonist; and (c) progesterone, a progesterone analogue, or a progesterone receptor agonist.

The amount of the insulin, estrogen, progesterone, or analogues or receptor agonists thereof present in the post-implantation medium can vary. For example, in some embodiments, the post-implantation medium can comprise about 1 ng/ml to about 100 mg/ml (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900 ng/ml, 1 μg/ml, 10, 20, 30, 40, 50, 60, 70, 80, 90, 100 μg/ml, 1 mg/ml, 10, 20, 30, 40, 50, 60, 70, 80, 90, 100 mg/ml or a number or a range between any two of these values) of one or more hormones (e.g., progesterone) and/or one or more growth factors (e.g., insulin or an insulin-like growth factor). In some embodiments, the post-implantation medium can comprise about 0.5 nM to about 1 mM (e.g., about 0.5, 1, 1.5, 2, 2.5, 3, 3.5, 4, 4.5 5 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900 nM, 0.5 mM, 1 mM, or a number or a range between any two of these values) of a hormone (e.g., estrogen) and/or insulin or an insulin-like growth factor.

In some embodiments, the insulin receptor agonist is selected from the group comprising IGF-I, IGF-II, analogues thereof, or any combination thereof. The estrogen receptor agonist can be selected from the group comprising β-estradiol, estrone, estriol and estetrol, or any analogue thereof. The post-implantation medium can comprise transferrin, sodium selenium, ethanolamine, or any analogue thereof. The post-implantation medium can comprise Insulin-Transferrin-Selenium-Ethanolamine (ITS-X). In some embodiments, the post-implantation medium further comprises an agonist of the activin type 1 or type 2 receptors. In some embodiments, the post-implantation medium does not comprise a reducing agent.

In some embodiments, the post-implantation culture medium may comprise a basal medium, as defined above (e.g., Advanced DMEM/F12) supplemented with, an insulin receptor agonist, e.g., Insulin (e.g., about 2 mg/ml to about 25 mg/ml), Transferrin (e.g., about 1 mg/ml to about 10 mg/ml), Selenium e.g., sodium selenite (e.g., about 0.001 mg/ml to about 0.01 mg/ml), Ethanolamine (e.g., about 0.5 mg/ml to about 10 mg/ml), an estrogen receptor agonist e.g., estradiol (e.g., about 5 nM to about 10 nM), and a progesterone receptor agonist e.g., Progesterone (e.g., about 50 ng/ml to about 500 ng/ml).

The post-implantation medium can also comprise an effective amount of a non-essential amino acid selected from the group comprising L-glycine, L-alanine, L-asparagine, L-aspartic acid, L-glutamic acid, L-proline and L-serine. The post-implantation medium can also comprise an effective amount of an essential amino acid selected from the group comprising L-histidine, L-isoleucine, L-leucine, L-lysine, L-methionine, L-phenylalanine, L-threonine, L-tryptophan and L-valine. The non-essential and/or essential amino acids can have an effective amount of, for example, about 0.1% to about 2% (e.g., about 0.1%, 0.2%, 0.3%, 0.4%, 0.5%, 1%, 1.1%, 1.2%, 1.3%, 1.4%, 1.5%, 1.6%, 1.7%, 1.8%, 1.9%, 2%, or a number or a range between any two of these values) volume per volume (% v/v), weight per volume (% w/v) or weight per weight (% w/w) of the medium. In some embodiments, the post-implantation medium comprises about 1% non-essential amino acids and/or essential amino acids.

The post-implantation medium can comprise L-glutamine. L-glutamine may be included in the culture medium at a concentration of about 0.1 mM to about 40 mM, about 0.2 mM to about 20 mM, about 0.5 mM to about 10 mM, about 1 mM to about 5 mM or about 1.5 mM to about 2.5 mM e.g., about 2 mM. In some embodiments, L-glutamine is included in the culture medium at a concentration of about 2 mM.

Penicillin may be included in the post-implantation culture medium at a concentration of about 1 unit/ml to about 500 units/ml, about 2 units/ml to about 250 units/ml, about 5 units/ml to about 100 units/ml, about 10 units/ml to about 50 units/ml, or about 20 units/ml to about 30 units/ml e.g., about 25 units/ml. Streptomycin may be included in the culture medium at a concentration of about 1 μg/ml to about 500 μg/ml, about 2 μg/ml to about 250 μg/ml, about 5 μg/ml to about 100 μg/ml, about 10 μg/ml to about 50 μg/ml, 25 or about 20 μg/ml to about 30 μg/ml e.g., about 25 μg/ml. The culture medium can comprise penicillin at a concentration of about 25 units/ml and/or streptomycin at a concentration of about 25 μg/ml. In some embodiments, the post-implantation culture medium can further comprise an antimicrobial agent, such as sodium lactate.

The post-implantation culture medium can also comprise an effective amount of glucose. The glucose can be included in the culture medium at a concentration of about 0.5 mM to about 5 mM, about 1 mM to about 4 mM, about 1.5 mM to about 3 mM, e.g., about 2 mM. In some embodiments, glucose is included in the culture medium at a concentration of about 1.8 mM.

In some embodiments, the post-implantation culture medium comprises DMEM/F12, fetal bovine serum, GlutaMax, essential and non-essential amino acids, ITS-X, β-estrodiol, progesterone, glucose, sodium lactate, penicillin and/or streptomycin, or any combination thereof. In some embodiments, the post-implantation culture medium comprises DMEM/F12, about 20% fetal bovine serum, about 1× GlutaMax, about 1× non-essential amino acids, about 1× essential amino acids, about 1×ITS-X, about 25 U/mL penicillin and/or streptomycin, about 1.8 nM Glucose, about 0.22% sodium lactate, about 8 nM O-estrodiol, about 200 ng/ml progesterone, or any combination thereof.

### Applications

Provided herein also includes a synthetic embryo model obtainable by the in vitro method described herein for use in a method of diagnosing, preventing or treating a disease in a patient in need thereof. For example, embryo cells obtainable from the present invention may be used in stem cell therapies, such as treatments for cancers, replacement tissue, reconstructive surgery, tissue repair, wound healing, bone marrow transplantation, stroke, baldness, blindness, deafness, diabetes, heart disease, bowel disease, arthritis, skeletal injury, teeth replacement, neuronal disease and any other condition where replacement cells or tissues may be advantageous. The cells may also be utilized for screening therapeutic compounds for efficacy and safety, as would be understood by a person of skill in the art.

In some embodiments, the synthetic embryo structure for use in a method of diagnosing, preventing or treating a disease in a patient in need thereof as described herein may be used for transplantation into the patient. It is envisaged that in certain embodiments, the pluripotent stem cell used to obtain the embryo may have been obtained from the patient originally, thus reducing the likelihood of rejection by the patient's immune system. Thus, a pluripotent stem cell, for example an embryonic stem cell, obtained from a patient may be cultured using the methods described herein to provide material for transplantation back into that patient to prevent or treat a condition. For example, the embryo may be used to grow replacement organs or tissues for the patient to regain function of such organs or tissues in the patient following loss of function through degeneration, ageing and/or disease.

Disclosed herein also includes a method of providing a transgenic non-human animal, comprising gestating an embryo derived from a cell cultured using an in vitro method described herein. Such transgenic non-human animals may be useful in drug screening or in the study of disease. For example, model animals may be produced to study specific conditions. It is envisaged that the methods provided herein could be used to more efficiently develop transgenic and chimeric embryos (which currently relies for example, on the labor-intensive process of harvesting blastocysts and manually replacing the inner cell mass).

Disclosed herein include methods for investigating the effect of a test agent on embryonic development. In some embodiments, the method comprises: a) generating a synthetic embryo model using the method described herein; b) contacting the synthetic embryo model with a test agent; and c) determining the effect of the test agent on the synthetic embryo model. In some embodiments, the determining comprises comparing a phenotype or a genotype of the synthetic embryo in the presence of the test agent with the phenotype or genotype of the synthetic embryo in the absence of the test agent. The method can comprise contacting the mammalian pluripotent stem cells (e.g., wild type and modified ESCs) with the test agent during or following step (a) and prior to step (b), during or following step (b) and prior to step (c), or during or following step (c).

The method can comprise determining the subsequent effect on formation of a synthetic embryo at various developmental stages. The determining can be performed using any method known in the art. For example, the method can comprise recording one or more images of the embryo structure.

Disclosed herein include methods for investigating mechanisms involved in embryogenesis. In some embodiments, the method comprises any of the in vitro methods for generating a synthetic embryo structure at various developmental stages described herein. Investigating mechanism involved in embryogenesis can comprise any method known in the art. For example, said investigating can comprise investigating the effect of a test agent on embryonic development as described above. In some embodiments, investigating mechanisms involved in embryogenesis can comprise determining the effect of genetic perturbation(s) in the embryo structure.

The method may comprise recording a plurality of images of the synthetic embryo structure. The plurality of images may be recorded over a pre-determined period of time, thus illustrating the development of the embryonic structure over time. The imaging apparatus may comprise microscopy apparatus, suitable recording apparatus, and optionally image processing apparatus.

Typically, fluorescent markers, such as fluorescent dyes or fluorescent marker proteins, are used in the imaging of embryonic development. Such markers may be added to the culture system. For example, fluorescent dyes may be added to visualize particular molecules or cellular structures. For example, DAPI may be used to stain DNA or MitoTracker (Invitrogen) may be used to stain the mitochondria. Additionally or alternatively, the embryo structure may produce such fluorescent markers endogenously, e.g., it may contain one or more cells which express a fluorescent marker protein. Such cells may have been genetically modified in order to confer the ability to express such a marker protein. Thus, fluorescence imaging apparatus may be particularly suitable for the methods described. The imaging apparatus may thus comprise a fluorescence microscope, such as a confocal microscope, that can include but is not limited to wide field, scanning and spinning disc confocal, and light sheet microscope.

Confocal microscopes image a single point of a specimen at any given time but allow generation of two dimensional or three-dimensional images by scanning different points in a specimen in a regular raster to provide image data which can be assembled into a two- or three-dimensional image. For example, scanning a specimen in a single plane enables generation of a two-dimensional image of a slice through the specimen. A plurality or “stack” of such two-dimensional images can be combined to yield a three-dimensional image. Spinning disc confocal microscopy provides added advantages over confocal laser scanning microscopy. Additionally, light sheet microscopy can also provide good imaging of embryonic development.

Disclosed herein also includes a method of elucidating the role of a gene in embryo development, the method comprising obtaining a pluripotent stem cell where the gene has been modified or knocked out and culturing the pluripotent stem cell (e.g., ESCs) and extraembryonic-like cells generated by transcription factor overexpression with the ESC using the in vitro method described herein. Thus, the methods may aid in the development of treatments for conditions relating to embryo development, such as fertility treatment.

Disclosed herein also includes a method of imaging an embryo during development comprising culturing a mammalian pluripotent stem cell (e.g., ESC) and mammalian extraembryonic-like cells generated by transcription factor overexpression with the ESC or a mammalian synthetic embryo structure using the methods described herein, and recording an image of said embryo using an imaging apparatus. The image may be a two dimensional or three-dimensional image. A plurality of images may be recorded of the same embryo. An imaging apparatus can comprise microscopy apparatus and suitable recording apparatus. An imaging apparatus may further comprise image processing apparatus. Additionally, an imaging apparatus may further comprise a fluorescent microscope. Additionally, or alternatively, an imaging apparatus may further comprise a confocal microscope.

### EXAMPLES

Some aspects of the embodiments discussed above are disclosed in further detail in the following examples, which are not in any way intended to limit the scope of the present disclosure.

### Materials and Methods

The following experimental materials and methods were used for Examples 1-5 described below.

### Ethics Statement

Work with human embryonic stem cells (Shef6) was carried out with approval from the UK Human Stem Cell Bank Steering Committee under approval SCSC21-38 and adhered to the regulations of the UK Code of Practice for Use of Human Stem Cell Lines. Human embryo work was regulated by the Human Fertility and Embryology Authority (HFEA) and carried out under license R0193. Ethical approval was obtained from the Human Biology Research Ethics Committee at the University of Cambridge. Patients undergoing IVF at CARE Fertility, Bourn Hall Fertility Clinic, Herts & Essex Fertility Clinic, and King's Fertility were given the option of continued storage, disposal, or donation of embryos to research (including project specific information) or training at the end of their treatment. Patients were offered counseling, received no financial benefit, and could withdraw their participation at any time until the embryo had been used for research. Informed research consent for donated embryos was obtained from both gamete providers. Only blastocysts that exhibited appropriate morphology (i.e. expanded blastocoel cavity and healthy inner cell mass) were used for subsequent experimentation. Embryos were not cultured beyond 14 d.p.f. or the first appearance of the primitive streak. Mice were kept in an animal house on 12:12 hour light-dark cycle with ad libitum access to food and water. Experiments with mice were regulated by the Animals (Scientific Procedures) Act 1986 Amendment Regulations 2012 and conducted following ethical review by the University of Cambridge Animal Welfare and Ethical Review Body (AWERB). Experiments were approved by the Home Office. CD1 and F1 wildtype males aged 6 to 45 weeks and CD1 and F1 wildtype females aged 6 to 18 weeks were used for this study. Animals were inspected daily. Those animals showing health concerns were culled by cervical dislocation. All embryo and embryoid works were performed in UK, and adhered to the 2021 ISSCR guidelines.

hESC Culture

Shef6 (from the UK Stem Cell Bank) or RUES2 (kindly provided by Ali Brivanlou, The Rockefeller University) human ESCs were cultured on Matrigel-coated plates in mTESR medium (05825, STEMCELL Technologies) at 37° C., 20% 02, 5% CO2. Plates were coated with 1.6% growth-factor reduced Matrigel (356230, BD Biosciences) dissolved in DMEM/F12 (21331-020, Life Technologies) for 1 hour at 37° C. hESCs were passaged with TrypLE (12604013, ThermoFisher Scientific). For the first 24 hours after passaging, 10 μM ROCK inhibitor Y-27632 (72304, STEMCELL Technologies) was added. Medium was changed every 24 hours. Cells were routinely tested for Mycoplasma contamination by PCR (6601, Takara Bio), and have been authenticated by short tandem repeat analysis. To convert primed hESCs to RSeT or PXGL culture conditions, cells were passaged to mitomycin-C inactivated CF-1 MEFs (3×103 cells/cm²; GSC-6101G, Amsbio) in media consisting of DMEM/F12 with 20% Knockout Serum Replacement (10828010, ThermoFisher Scientific), 100 μM β-mercaptoethanol (31350-010, Thermo Fisher Scientific), 1× GlutaMAX (35050061, Thermo Fisher Scientific), 1× non-essential amino acids, 1× penicillin-streptomycin and 10 ng/ml FGF2 (Department of Biochemistry, University of Cambridge) and 10 μM ROCK inhibitor Y-27632 (72304, STEMCELL Technologies). For RSeT cells, media was switched to RSeT media after 24 hours (05978, STEMCELL Technologies). Cells were maintained in RSeT and passaged as above every 4-5 days. For PXGL cells, conversion was performed as previously described. Briefly, cells were cultured in 5% 02, 7% CO2. Media was switched to chemical resetting media 1 (cRM-1) consisting of N2B27 media supplemented with 1 μM PD0325901 (University of Cambridge, Stem Cell Institute), 10 ng/mL human recombinant LIF (300-05, PeproTech), and 1 mM Valproic Acid. N2B27 contained 1:1 DMEM/F12 and Neurobasal A (10888-0222, Thermo Fisher Scientific) supplemented with 0.5×B27 (10889-038, Thermo Fisher Scientific), 0.5×N2 (made in-house), 100 μM β-mercaptoethanol, 1× GlutaMAX, and 1× penicillin-streptomycin. cRM-1 media was changed every 48 hours for 4 days, after which media was changed to PXGL. PXGL media consisted of N2B27 supplemented with 1 μM PD0325901, 10 ng/mL human recombinant LIF, 2 μM G66983 (2285, Tocris) and 2 μM XAV939 (X3004, Merck). PXGL cells were passaged every 4-6 days using TrypLE (12604013, Thermo Fisher Scientific) for 3 min. 10 μM ROCK inhibitor Y-27632 and 1 μL/cm² Geltrex (A1413201, Thermo Fisher Scientific) were added at passage for 24 hours. For yolk sac-like cell or trophoblast differentiation, RSeT cells were passaged onto matrigel-coated IBIDI chamber slides. 24 hours later, media was switched to ‘ACL’ (100 ng/ml Activin-A, Qk001, QKINE, 3 μM CHIR99021, 72052, STEMCELL Technologies, and 10 ng/ml human LIF) for hypoblast induction or ‘PA’ (1 μM PD0325901 and 1 μM A83-01, 72022, STEMCELL Technologies) with or without 500 nM lysophosphatidic acid—LPA (3854, Tocris).

Generation of Inducible hESC Lines

To generate piggyback plasmids, full-length coding sequences were amplified from human cell line cDNA with AttB overhangs using Phusion High-Fidelity DNA polymerase (M0530S, New England BioLabs) according to manufacturer's instructions. Amplicons were introduced to pDONR221 entry plasmids using BP clonase (11789100, ThermoFisher Scientific), and subsequently to destination plasmids using LR clonase (11791020, Thermo Fisher Scientific) according to manufacturer's instructions. hESCs were electroporated with GATA6-3×FLAG-TetOn-Zeo (entry plasmid 72922, Addgene) and/or SOX17-TetOn-Hygro or GATA3-EGFP-TetO-Hygro and/or TFAP2C-TetOn-G418 in addition to PB-CAG-rTTA3-Bsd or PB-CAG-rTTA3-Zeo and pBase plasmid expressing PiggyBac Transposase using the Neon transfection system with the following settings: 1200 V, 20 ms, and 2 pulses. Two days after transfection, antibiotics were applied at a ¼ dosage and increased to final concentrations of 100 μg/mL zeocin (ant-zn-1, Invitrogen), 20 μg/mL blasticidin (A113903, ThermoFisher Scientific), 50 μg/mL G418 (10131035, ThermoFisher Scientific) or 50 μg/mL HygromycinB (10687010, ThermoFisher Scientific). Shef6-mKate2 hESCs were obtained as a gift. Clones were generated by manually picking single colonies under a dissecting microscope. Transgene activation was triggered by the addition of 1 μg/mL doxycycline hyclate (D9891, Sigma). To select clones for downstream experiments, isolated colonies that survived manual picking were induced for 72 hours and cell pellets were collected for qPCR or stained for immunofluorescent analysis. Immunofluorescent analysis was performed in primed hESCs. Transgene expression and another key lineage marker were assessed for changes in expression compared to uninduced controls. Clones with robust transgene upregulation and downstream upregulation of an uninduced lineage marker were selected for subsequent experimentation (e.g. 1-2 clones per transgenic line). Note that AP2γ-inducible cells failed to reset in PXGL naïve conditions.

qRT-PCR Analysis

Cell pellets were harvested and RNA was extracted using the Qiagen RNeasy kit following manufacturer's instructions. Reverse transcriptase reaction was performed with 1 μg RNA with random primers (C1181, Promega), dNTPs (N0447S, New England BioLabs), RNAse inhibitor (M0314L, New England Biolabs), and M-MuLV reverse transcriptase (M0253L, New England Biolabs). RT-qPCR was performed using Power SYBR Green PCR Master Mix (4368708, ThermoFisher Scientific) on a Step One Plus Real-Time PCR machine (Applied Biosystems). The following program was used: 10 minutes at 95° C. followed by 40 cycles of 15 seconds at 95° C. and 1 minute at 60° C. Single melt curves were observed for all primers used in this study. Oligonucleotides used in this study are provided in Table 2 below.

TABLE 2
THE OLIGONUCLEOTIDES USED IN THIS STUDY

| Primer | Sequence | SEQ ID NO: |
|---|---|---|
| qPCR_GATA6_Fwd | CTCAGTTCCTACGCTTCGCAT | 1 |
| qPCR_GATA6_Rev | GTCGAGGTCAGTGAACAGCA | 2 |
| qPCR_SOX17_Fwd | TTCGTGTGCAAGCCTGAGAT | 3 |
| qPCR_SOX17_Rev | TAATATACCGCGGAGCTGGC | 4 |
| qPCR_PDGFRA_Fwd | TGGCAGTACCCCATGTCTGAA | 5 |
| qPCR_PDGFRA_Rev | CCAAGACCGTCACAAAAAGGC | 6 |
| qPCR_GATA4_Fwd | CGACACCCCAATCTCGATATG | 7 |
| qPCR_GATA4 Rev | GTTGCACAGATAGTGACCCGT | 8 |
| qPCR_CER1_Fwd | ACAGTGCCCTTCAGCCAGACT | 9 |
| qPCR_CER1_Rev | ACAACTACTTTTTCACAGCCTTCGT | 10 |
| qPCR_COL4A1_Fwd | GGGATGCTGTTGAAAGGTGAA | 11 |
| qPCR_COL4A1_Rev | GGTGGTCCGGTAAATCCTGG | 12 |
| qPCR_SOX2_Fwd | GAGCTTTGCAGGAAGTTTGC | 13 |
| qPCR_SOX2_Rev | GCAAGAAGCCTCTCCTTGAA | 14 |
| qPCR_CD48_Fwd | AGGTTGGGATTCGTGTCTGG | 15 |
| qPCR_CD48_Rev | AGTTGTTTGTAGTTCTCAGGCAG | 16 |
| qPCR_GATA3_Fwd | GCCCCTCATTAAGCCCAAG | 17 |
| qPCR_GATA3_Rev | TTGTGGTGGTCTGACAGTTCG | 18 |
| qPCR_TFAP2C_Fwd | TGCACGATCAGACAGTCATTC | 19 |
| qPCR_TFAP2C_Rev | GTAGAGCTGAGGAGCGACAATC | 20 |
| qPCR_GATA2_Fwd | ACTGACGGAGAGCATGAAGAT | 21 |
| qPCR_GATA2_Rev | CCGGCACATAGGAGGGGTA | 22 |
| qPCR_TFAP2A_Fwd | AGGTCAATCTCCCTACACGAG | 23 |
| qPCR_TFAP2A_Rev | GGAGTAAGGATCTTGCGACTGG | 24 |
| qPCR_TACSTD2_Fwd | ACAACGATGGCCTCTACGAC | 25 |
| qPCR_TACSTD2_Rev | AGTTCACGCACCAGCACAC | 26 |
| qPCR_KRT19_Fwd | ACCTGGAGATGCAGATCGAA | 27 |
| qPCR_KRT19_Rev | AATCCACCTCCACACTGACC | 28 |
| qPCR_GABRP_Fwd | TTTCTCAGGCCCAATTTTCCT | 29 |
| qPCR_GABRP_Rev | GCTGTCGGAGGTATATGGTGG | 30 |
| qPCR_ISL1_Fwd | GCGGAGTGTAATCAGTATTTGGA | 31 |
| qPCR_ISL1_Rev | GCATTTGATCCCGTACAACCT | 32 |
| TFAP2C-AttB_Fwd | GGGGACAAGTTTGTACAAAAAAGCAGGCTT | 33 |
| | CACCATGTTGTGGAAAATAACCGATA | |
| TFAP2C-AttB_Rev | GGGGACCACTTTGTACAAGAAAGCTGGGTCT | 34 |
| | TATTTCCTGTGTTTCTCCATT | |
| SOX17-AttB_Fwd | GGGGACAAGTTTGTACAAAAAAGCAGGCTT | 35 |
| | CACCATGAGCAGCCCGGATGC | |
| SOX17-AttB_Rev | GGGGACCACTTTGTACAAGAAAGCTGGGTCT | 36 |
| | CACACGTCAGGATAGTTGCAG | |

Generation of hPSC-Mouse Embryo Chimeras

For human cell-mouse embryo chimeras, oviducts and uterine horns were recovered and flushed with M2 medium (made in-house) supplemented with 4 mg/mL BSA (A9418, Sigma) on E2.5. Recovered pre-compacted eight-cell stage embryos were then subjected to zona pellucida removal by treatment with Acidic Tyrode's Solution. Human cells (wildtype, 3-day induced GATA6-50×17 cells, and 3-day induced GATA3-AP2γ RSeT cells) were prepared by dissociating cells with TrypLE and washing as described above. Cells were resuspended in either RSeT or N2B27 media supplemented with 5% KSR and 1 μg/mL doxycycline. The resulting small clumps of cells were aggregated with the 8-cell stage mouse embryos in indentations in these media for 24 hours, before being transferred to KSOM±1 μg/mL doxycycline for a further 24 hours until E4.5. For negative controls, embryos were cultured in these conditions without the addition of human cells. Chimeric blastocysts were then fixed for analysis by immunofluorescence. The contribution of human nuclear antigen-positive cells to either SOX2, SOX17, and/or GATA3 populations were quantified in those embryos that successfully developed to the late blastocyst stage.

### Generation of Inducible Human Embryoids

To generate the three-dimensional stem cell-derived model of the post-implantation embryo, RSeT cells between 2 and 6 passages post-conversion to RSeT media were passaged as normal. The media for extraembryonic-like cells (induced GATA6, induced GATA6-SOX17 or induced GATA3-AP2γ) was changed to N2B27 with 5% Knockout Serum Replacement and 1 μg/mL DOX on the following day (Day −3). This media was refreshed every 24 hours for 3 days. On Day 0 (the day of aggregation), an Aggrewell dish (34415, STEMCELL Technologies) was prepared by pre-coating with anti-adherence solution (07010, STEMCELL Technologies) and centrifuging at 2000 g for 5 minutes. Wells were washed twice with PBS prior to the addition of experiment media. This media consisted of N2B27 with 5% knockout serum replacement, 1 μg/mL doxycycline, and 10 μM Y-27632. Induced cells and wildtype ESCs were enzymatically dissociated 1 hour after addition of 10 μM Y-27632 to wells containing cells for inducible human embryoid generation. Dissociated cells were pelleted and resuspended in experiment media and placed in gelatin-coated wells for MEF depletion. After 15-30 minutes, cells were counted, mixed, and plated into an Aggrewell dish with a final calculation of 8 wildtype-ESC, 8 hypoblast-like, and 16 trophoblast-like cells plated per microwell in the Aggrewell. At 8 d.p.f., in vitro cultured human embryos had 32:24:228 epiblast:hypoblast:trophoblast cells. Importantly, however, many of the trophoblast cells were not in contact with the inner cell mass-derived tissues or were terminally differentiated. Additionally, inducible GATA6-SOX17 cells in culture proliferated slower than the other two cell populations after doxycycline addition. Therefore, an initial seeding density with: (1) a total cell number similar to that used in mouse models that allowed for successful cell sorting; (2) a ratio of cells that reflected the peri-implantation embryo; and (3) a reduced number of inducible GATA3-AP2γ cells and an increased number of inducible GATA6-SOX17 cells was utilized.

On Day 1, the media was subjected to two two-thirds changes of N2B27 with 5% knockout serum replacement and 1 μg/mL doxycycline. On Day 2, Aggrewells were subjected to a half change with hIVC1 media containing 25 ng/mL hIGF1 (78022.1 STEMCELL Technologies) and 1 μg/mL doxycycline. hIVC1 media consisted of Advanced DMEM/F12 (12634-010 Thermo Fisher Scientific) supplemented with 20% inactivated FBS (10270106, ThermoFisher Scientific), 1× Glutamax, 1×NEAA, 1× Essential AA, 1×ITS-X, 25 U/mL Pen/Strep, 1.8 mM Glucose (G8644, Sigma-Aldrich), 0.22% sodium lactate (L7900, Sigma-Aldrich), 8 nM β-estradiol (50-28-2, Tocris) and 200 ng/mL progesterone (P0130, Sigma-Aldrich). This media was used in half changes each day from Day 3. On Day 4, aggregates were manually picked using a mouth pipette under a dissecting microscope into individual wells of ultra-low attachment 96 well plates (CLS7007, Corning) in hIVC1 media with IGF1 and doxycycline as described above for subsequent culture.

### Immunostaining and Image Analysis

Samples were washed with phosphate-buffered saline (PBS) and fixed in 4% paraformaldehyde (PFA; 1710, Electron Microscopy Sciences) at room temperature for 20 minutes. Samples were washed 3 times with PBS containing 0.1% (vol/vol) Tween-20 (PBST) and incubated with 0.3% (vol/vol) Triton X-100 (T8787, Sigma Aldrich) with 0.1 mM glycine (BP381-1, Thermofisher Scientific) in PBS at room temperature for 30 minutes. Samples were blocked in blocking buffer (PBST with 5% (w/vol) BSA, A9418, Sigma), then incubated with primary antibodies diluted in blocking buffer overnight at 4° C. A list of primary antibodies is in Table 3 below. Samples were washed three times in PBST and incubated with fluorescently conjugated AlexaFlour secondary antibodies (Thermofisher Scientific, 1:500) and DAPI (D3571, ThermoFisher Scientific, 1 μg/mL) diluted in blocking buffer for 2 hours at room temperature. For pSMAD1.5 quantification, OCT4-positive or GATA6-positive nuclei were isolated and the fluorescent intensity of pSMAD1.5 was quantified. For SMAD2.3 quantification, OCT4-positive or GATA6-positive (excluding the outermost GFP+ cell layer) nuclei fluorescence intensity as well as cytoplasmic fluorescence intensity was quantified. Data were presented as the ratio of nuclear:cytoplasmic fluorescence intensity. Immunofluorescence images were analyzed using FIJI. The spots tool with manual curation in Imaris software (version 9.1.2, Oxford Instruments) was used to quantify total cell numbers in day 4 embryoids and generated spot renders.

TABLE 3
ANTIBODIES USED IN THIS STUDY

| Primary Antibody List | | | | | | |
|---|---|---|---|---|---|---|
| Target | Species | Company | Catalogue # | Clone # | RRID | Dilution |
| AP2-alpha | Mouse | Santa Cruz | sc-12726 | 3B5 | AB_667767 | 1:200 |
| | | Biotechnology | | | | |
| AP2-gamma | Goat | R&D Systems | AF5059 | polyclonal | AB_2255891 | 1:500 |
| AP2-gamma | mouse | Santa Cruz | sc-12762 | 6E4/4 | AB_667770 | 1:200 |
| | | Biotechnology | | | | |
| Brachyury | Goat | R&D Systems | AF2085 | polyclonal | AB_2200235 | 1:500 |
| CDX2 | Mouse | BioGenex | MU392-UC | CDX2-88 | AB_2335627 | 1:200 |
| Cerberus 1 | Goat | R&D Systems | AF1075 | polyclonal | AB_2077228 | 1:500 |
| Cytokeratin 7 | Mouse | Agilent | M7018 | OV-TL 12/30 | AB_2134589 | 1:100 |
| E-Cadherin | Mouse | BD Biosciences | 610182 | 36 | AB_39581 | 1:200 |
| EOMES | Rabbit | Abcam | ab23345 | polyclonal | AB_778267 | 1:200 |
| FOXA2 | Goat | R&D Systems | AF2400 | polyclonal | AB_2294104 | 1:200 |
| GATA2 | Rabbit | Novus Biologics | NBP1-82581 | polyclonal | AB_11026191 | 1:200 |
| GATA3 | Goat | Abcam | ab199428 | EPR16651 | AB_2819013 | 1:500 |
| GATA4 | Mouse | Santa Cruz | sc-25310 | G-4 | AB_627667 | 1:200 |
| | | Biotechnology | | | | |
| GATA4 | Rat | ThermoFisher | 14-9980-82 | eBioEvan | AB_763541 | 1:500 |
| | | Scientific | | | | |
| GATA6 | Goat | R&D Systems | AF1700 | polyclonal | AB_2108901 | 1:500 |
| GATA6 | Rabbit | Cell Signaling | 5851 | D61E4 | AB_10705521 | 1:2000 |
| | | Technology | | | | |
| GFP | Chicken | Abcam | ab13970 | polyclonal | AB_300798 | 1:1000 |
| GFP | Rat | Nacalai USA | GF090R | GF090R | AB_2314545 | 1:1000 |
| HAND1 | Mouse | DSHB | PCRP-HAND1- | 2A9 | AB_2618668 | 1:200 |
| | | | 2A9 | | | |
| HNF4a | Rabbit | Abcam | ab201460 | EPR16885-99 | AB_2927380 | 1:2000 |
| ISL1 | Mouse | DSHB | PCRP-ISL1- | 1A9 | AB_2618775 | 1:100 |
| | | | 1A9 | | | |
| Laminin | Rabbit | Sigma Aldrich | L9393 | polyclonal | AB_477163 | 1:200 |
| NANOG | Rabbit | Cell Signaling | 4903 | D73G4 | AB_10559205 | 1:200 |
| | | Technology | | | | |
| N-Cadherin | Mouse | Abcam | ab98952 | 5D5 | AB_10696943 | 1:200 |
| OCT3/4 | Mouse | Santa Cruz | sc-5279 | C-10 | AB_628051 | 1:100 |
| | | Biotechnology | | | | |
| OTX2 | Goat | R&D Systems | AF1979 | polyclonal | AB_2157172 | 1:1000 |
| phosp- | Rabbit | Cell Signaling | 9516S | 41D10 | AB_491015 | 1:200 |
| Smad1/5 | | Technology | | | | |
| Smad2/3 | Rabbit | Cell Signaling | 8685S | D7G7 | AB_10889933 | 1:200 |
| | | Technology | | | | |
| SOX17 | Goat | R&D Systems | AF1924 | polyclonal | AB_355060 | 1:500 |
| SOX2 | Rat | ThermoFisher | 14-9811-82 | Btjce | AB_11219471 | 1:500 |
| | | Scientific | | | | |
| TBX20 | Mouse | R&D Systems | MAB8124 | 668710 | AB_2255728 | 1:100 |
| VTCN1 | Rabbit | Abcam | ab209242 | EPR20236 | AB_2801513 | 1:200 |

| Secondary Antibodies | | | | | | |
|---|---|---|---|---|---|---|
| Target | Species | Fluorophore | Company | Catalogue # | RRID | Dilution |
| Mouse | Donkey | 405 | ThermoFisher | A48257 | AB_2884884 | 1:500 |
| | | | Scientific | | | |
| Rat | Donkey | 488 | ThermoFisher | A-21208 | AB_2535794 | 1:500 |
| | | | Scientific | | | |
| Mouse | Donkey | 488 | ThermoFisher | A-21202 | AB_141607 | 1:500 |
| | | | Scientific | | | |
| Goat | Donkey | 488 | ThermoFisher | A-11055 | AB_2534102 | 1:500 |
| | | | Scientific | | | |
| Rabbit | Donkey | 568 | ThermoFisher | A10042 | AB_2757564 | 1:500 |
| | | | Scientific | | | |
| Rat | Donkey | 568 | ThermoFisher | A78946 | AB_2910653 | 1:500 |
| | | | Scientific | | | |
| Mouse | Donkey | 568 | ThermoFisher | A10037 | AB_2534013 | 1:500 |
| | | | Scientific | | | |
| Rat | Donkey | 647 | ThermoFisher | A78947 | AB_2910635 | 1:500 |
| | | | Scientific | | | |
| Goat | Donkey | 647 | ThermoFisher | A-21447 | AB_141844 | 1:500 |
| | | | Scientific | | | |
| Rabbit | Donkey | 647 | ThermoFisher | A-31573 | AB_2536183 | 1:500 |
| | | | Scientific | | | |
| Mouse | Donkey | 647 | ThermoFisher | A32787 | AB_2762830 | 1:500 |
| | | | Scientific | | | |

### Human Embryo Thawing and Culture

Human embryos were thawed and cultured as described previously. Briefly, cryopreserved human blastocysts (5 or 6 d.p.f.) were thawed using the Kitazato thaw kit (VT8202-2, Hunter Scientific) according to the manufacturer's instructions. The day prior to thawing, thawing solution (TS) was placed at 37° C. overnight. The next day, in vitro fertilization (IVF) straws were submerged in 1 mL pre-warmed TS for 1 min. Embryos were then transferred to diluent solution (DS) for 3 min, washing solution 1 (WS1) for 5 min, and washing solution 2 (WS2) for 1 min. These steps were performed in reproplates (REPROPLATE, Hunter Scientific) using a STRIPPER micropipette (Origio). Embryos were incubated at 37° C. and 5% CO2 in normoxia and in pre-equilibrated human IVC1 supplemented with 50 ng/mL Insulin Growth Factor-1 (IGF1) (78078, STEMCELL Technologies) under mineral oil for 1-4 hours to allow for recovery. Following thaw, blastocysts were briefly treated with acidic Tyrode's solution (T1788, Sigma) to remove the zona pellucida and placed in pre-equilibrated post-implantation human embryo media (hIVC1) in 8 well μ-slide tissue culture plates (80826, Ibidi) in approximately 400 μL volume per embryo per well. Half media changes were done every 24 hours.

### Statistical Analysis

Statistical analyses were performed using Graphpad Prism v9.4. Sample sizes were not predetermined, and the researchers were not blinded to conditions. All experiments were performed independently at least twice. Data were tested for normality using the Shapiro-Wilk test. Normally distributed data was analyzed using parametric tests (unpaired t-test or ANOVA) and non-normally distributed data was analyzed using non-parametric tests (Mann Whitney U or Kruskal Wallis tests) as indicated in figure description. Numbers of samples are indicated in figure description. All statistical tests were two-tailed. For each test, individual samples were used. Replicates are biological unless otherwise stated. Within plots, all data were presented as mean±SEM. For box plots, the box represents the 25^{th}-75^{th} quartiles and whiskers represent minimum and maximum, with the central line representing median and + symbol representing mean. For multiple comparisons testing, comparisons were only made to the control condition. Unmarked pairwise comparisons were not significant (p>0.05).

### Collection, Generation, and Sequencing of Single-Nuclei ATAC/RNA 10× Libraries

To collect post-implantation embryo-like models for single-cell sequencing, correctly organized embryoids on Days 4, 6, and 8 were picked visually and washed through PBS twice in a 4-well dish before being transferred to TrypLE. Samples were agitated by pipetting every 5 minutes for 10-20 minutes until dissociated. Enzymatic activity was inactivated through addition of 20% fetal bovine serum (FBS) in PBS at 2× volume. Cells were collected in a falcon tube, pelleted, and resuspended in freeze buffer consisting of 50 mM Tris at pH 8.0 (15-567-027, Fisher Scientific), 25% glycerol (G5516, Sigma-Aldrich), 5 mM Mg(OAc)₂ (63052, Sigma-Aldrich), 0.1 mM EDTA (15575020, ThermoFisher Scientific), 5 mM DTT (R0861, ThermoFisher Scientific), 1× protease inhibitor cocktail (P8340, Sigma-Aldrich), and 1:2500 dilution of superasin (AM2694, Invitrogen). For cell lines, 10,000 cells were counted, pelleted, and resuspended in the freeze media described above before being slow-frozen at −80° C.

For nuclei isolation and library construction, low input nuclei isolation protocol from 10× Genomics was performed. Briefly, frozen cell pellets were thawed in a 37° C. water bath for 30 seconds, and centrifuged (500 g for 5 minutes at 4° C.) to pellet the cells. Then, the supernatant was aspirated. The cell pellets were washed twice with 200 μL 1×PBS with 0.04% BSA, and centrifuged. Supernatant was aspirated between washes. Subsequently, chilled lysis buffer (45 μL per sample) was added to the washed cell pellet. The cell pellet with lysis buffer was placed on ice for 3 minutes. Then, wash buffer (50 μL per sample) was added. Washed isolated nuclei were resuspended in a diluted nuclei buffer. The isolated nuclei were resuspended in 5 μL of diluted nuclei buffer and were directly added to the transposition reaction. In all following steps, 10× Genomics' Single Cell Multiome ATAC and Gene Expression protocol were followed according to manufacturer's specifications and guidelines. The final libraries were loaded on the NextSeq 2000 using P2 100 cycle kit at 650 μM loading concentration with paired-end sequencing following the recommended sequencing reads from 10× Genomics (28/10/10/90 cycles for gene expression libraries and 50/8/24/49 cycles for ATAC libraries).

### Single Cell Sequencing Analysis

Processing and Quality Control

Raw reads were analyzed using the CellRangerARC pipeline to generate ATAC and RNA fastq files for each sample, and then to align genomic and transcriptomic reads. Matrices were then read into Seurat48 and Signac49 using the Read10X_h5 command. For ATAC-seq data, peaks from standard chromosomes were used and peaks were additionally called using macs2 to add an additional Signac assay. Cells with >500 RNA UMI counts, <20% mitochondrial reads, >500 ATAC reads, TSS enrichment >1 and that were called as singlets using scDblFinder50 were retained for downstream analysis. For UMAP projections, SCTransform was used for RNA counts with percent mitochondrial counts and cell cycle scores regressed. PCA and LSI graphs were used to generate a weighted nearest neighbor (wnn) embedding, which accounted for both modalities. chromVAR51 was run to calculate motif accessibility score on the peaks assay. Data visualization was performed using Seurat's DimPlot, FeaturePlot, VlnPlot, TSSPlot, FragmentHist functions as well as SCpubr's52 do_Alluvialplot and do_Nebulosaplot functions.

Comparisons to Published Datasets

Analytical tool, scmap, was used to project cell labels from other single cell datasets onto post-implantation embryo-like model transcriptional data. All reference data used were publicly available with published cell type annotations. Cynomolgus monkey gene names were converted to hgnc gene symbols using biomaRt. For data generated with smart-seq2 or other non-UMI-based single cell sequencing methods, the scmapCluster method was used with a similarity threshold of 0.5. For UMI-based methods the scmapCell followed by scmapCell2Cluster method was used with w=2. Multiple datasets were used to draw conclusions with scmap. Transcriptionally similar clusters (e.g. trophoblast or amnion) may be mapped incorrectly if both are not present due to the limited cell assignments in certain datasets. Upon cell type assignment and processing for the cell lines sequenced, a previously reported and validated logistic regression framework was applied to project cell line data onto published single cell data and to project published cluster annotations (e.g. training data) onto post-implantation embryo-like model clusters (e.g. test data), resulting in a quantitative measure of predicted similarities. Here, only differentially expressed genes (produced using Seurat's FindAllMarkers function on course cell assignments, which collapsed amnion and mesodermal clusters) were used.

Multivelo RNA/Chromatin Velocity

A recently published method for velocity calculations, Multivelo, that accounted for both single cell ATAC and RNA data was applied. Multivelo was run on all cells, which passed the QC and processing described above. Analysis was based on available vignettes with 1000 highly variable genes and the ‘grid’ method. Gene expression and chromvar was plotted over latent time using the switched package.

CellPhoneDB Analysis

CellPhoneDB 2.0 was used with default settings to assess potential tissue signaling crosstalk. Course cell assignments, which collapsed separated amnion (AM-1, AM-2, AM-3) and mesoderm (MESO-1, MESO-2) clusters, was used for simplicity. Selected significant interactions were plotted as dot plots.

Reanalysis of Human In Vitro Cultured Embryo Datasets

Previously published data were realigned to the hg38 human genome using kallisto or kb-bustools. Datasets which were not sequenced with UMI-based technologies were normalized using quminorm to quasiumis. Using SCTransform-based integration, datasets were combined to generate a single-cell RNA-seq dataset of human embryos spanning zygote to day 14 post-fertilization. Cells were clustered and identities assigned based on previous annotations and canonical marker expression. The dataset showed good overlap with datasets with separation of cell types and some temporal resolution. SCENIC was used with default settings in R, and the AUC-regulon table was used to generate a new assay in the Seurat object. Using this assay, the epiblast, hypoblast and trophoblast lineages were then compared using Seurat's FindMarkers function to implement a Wilcoxon ranked test with Bonferroni correction to identify pairwise predicted differentially active regulons. Regulons that were enriched across both relevant comparisons (e.g. hypoblast versus epiblast; hypoblast versus trophoblast) were used as enriched active transcription factors for subsequent analyses (e.g. in the hypoblast). These factors were then plotted in relation to each other in Cytoscape.

### Data and Code Availability

For aligning sequencing data, GRCh38 (www.ncbi.nlm.nih.gov/assembly/GCF_000001405.26/) and GRCm38 (www.ncbi.nlm.nih.gov/assembly/GCF_000001635.20/) were used. Code used to analyze data mentioned herein is available at //github.com/bweatherbee/human_model.

### Example 1

### Induction of Extraembryonic Lineages

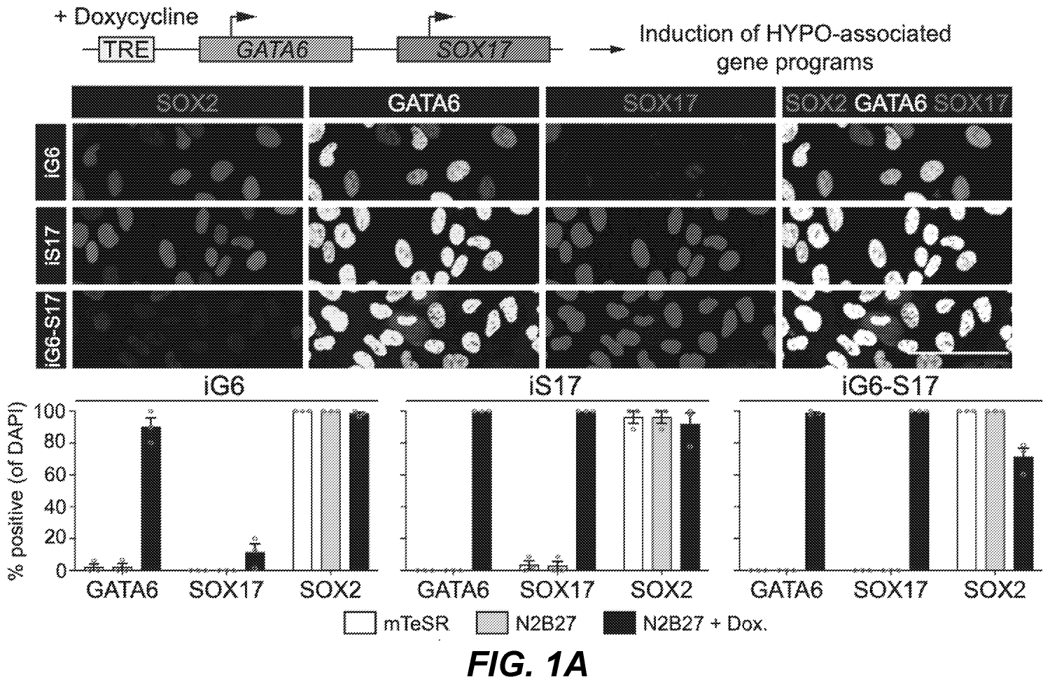

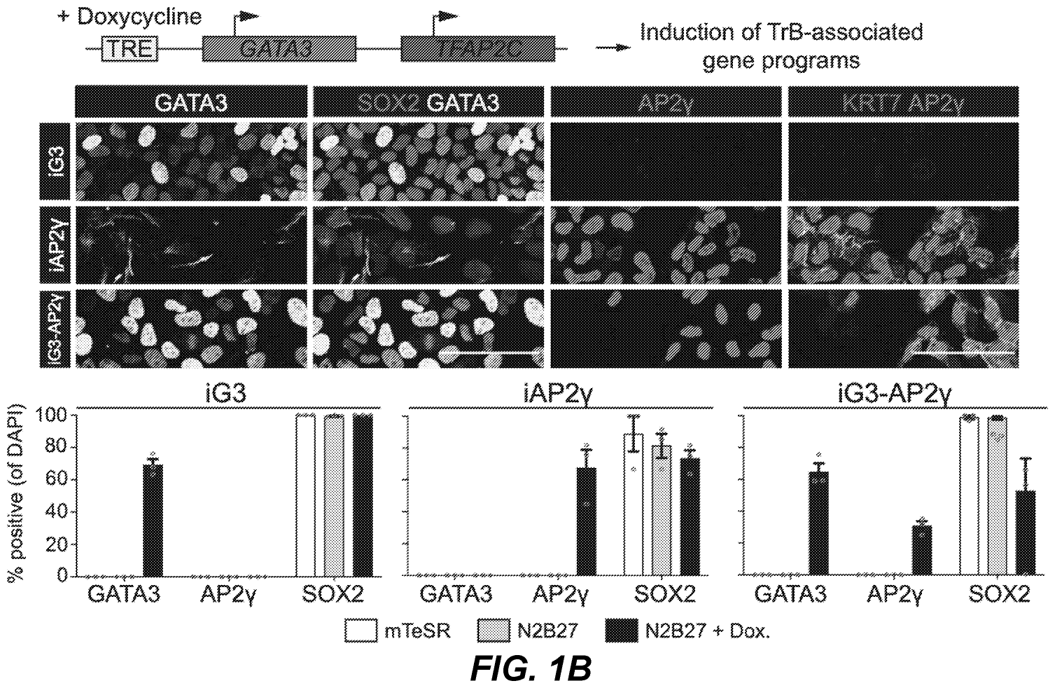

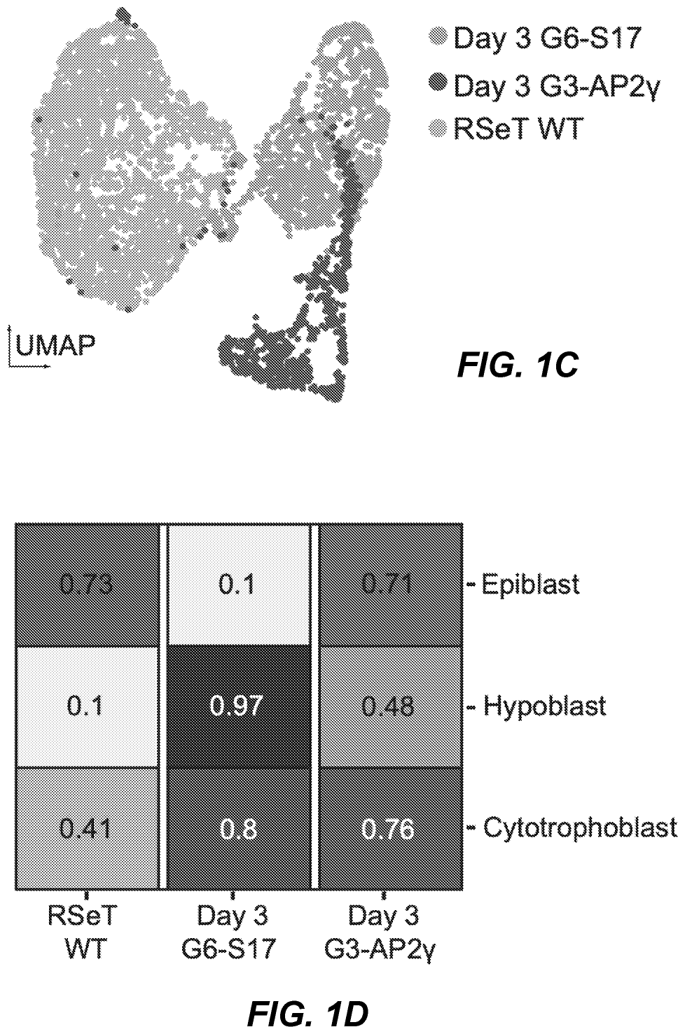

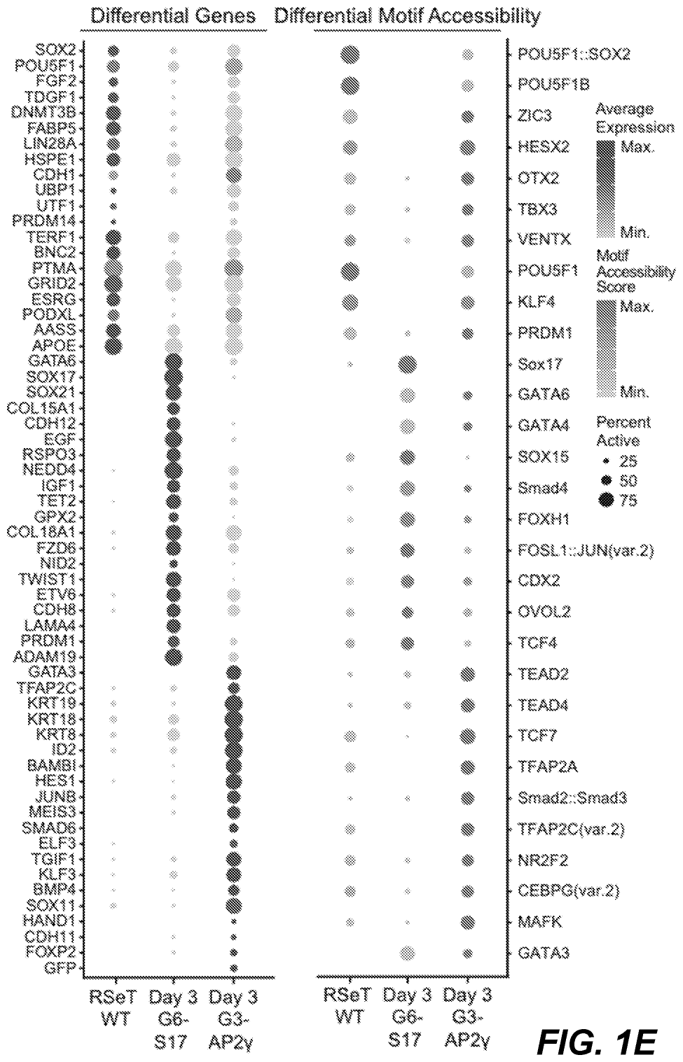

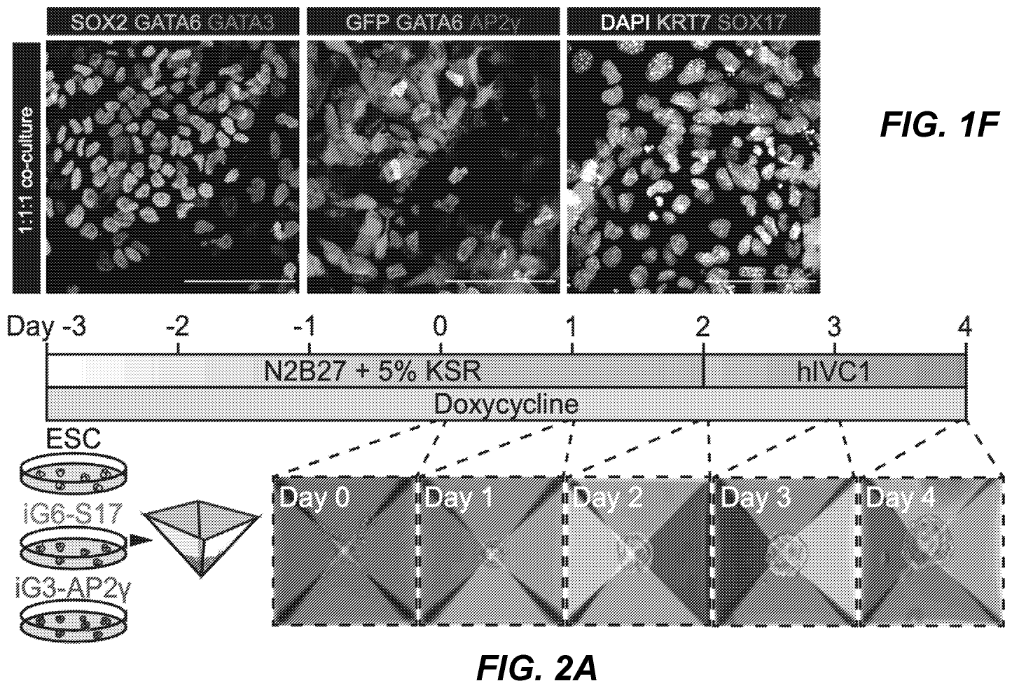

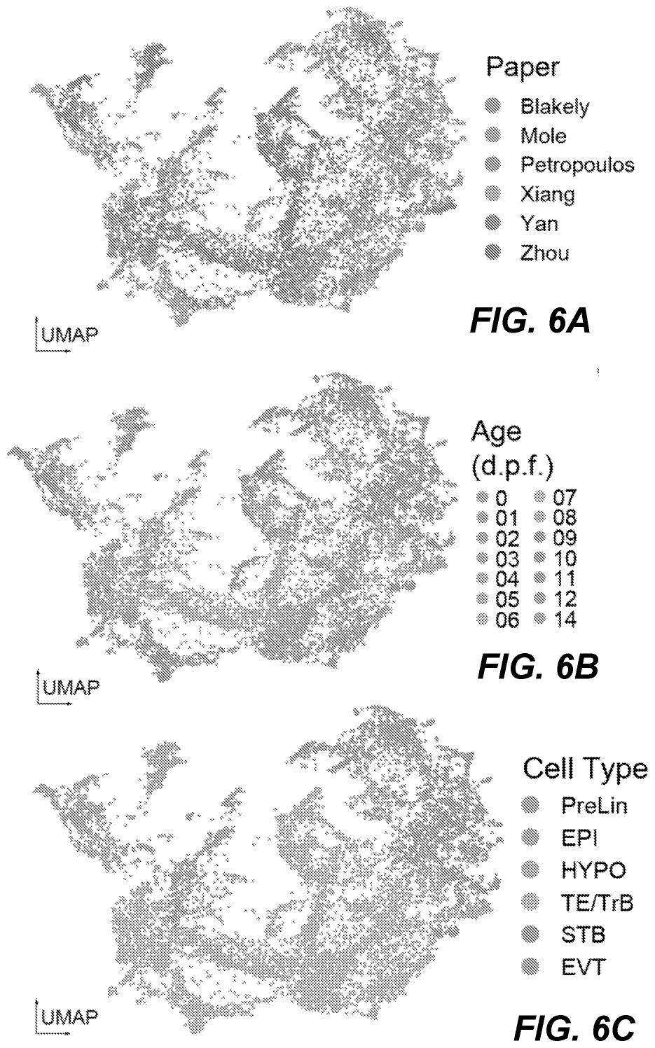

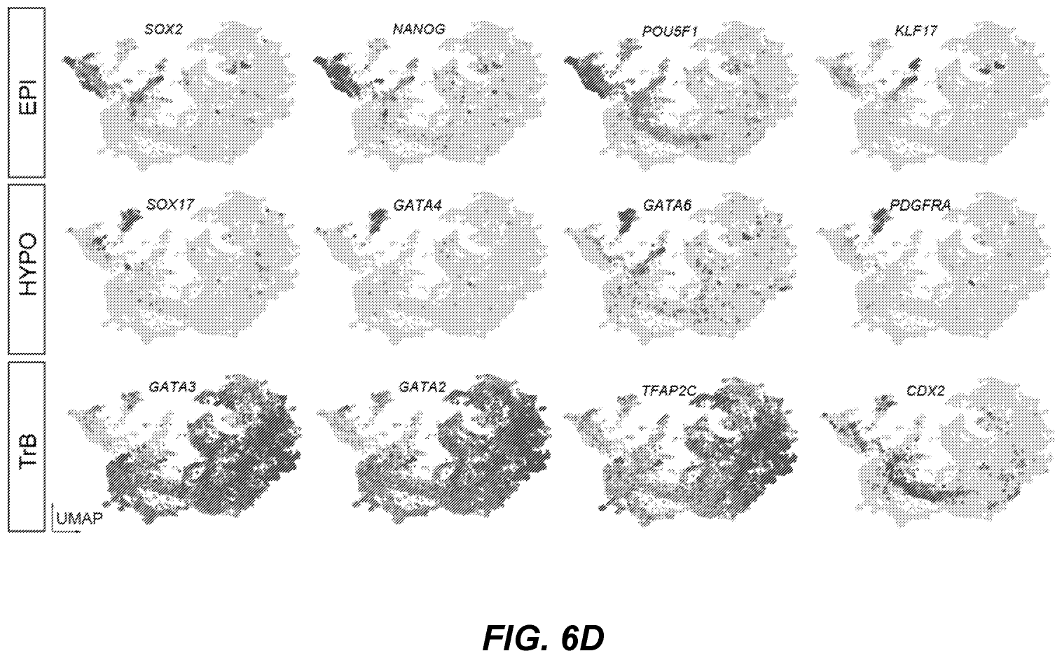

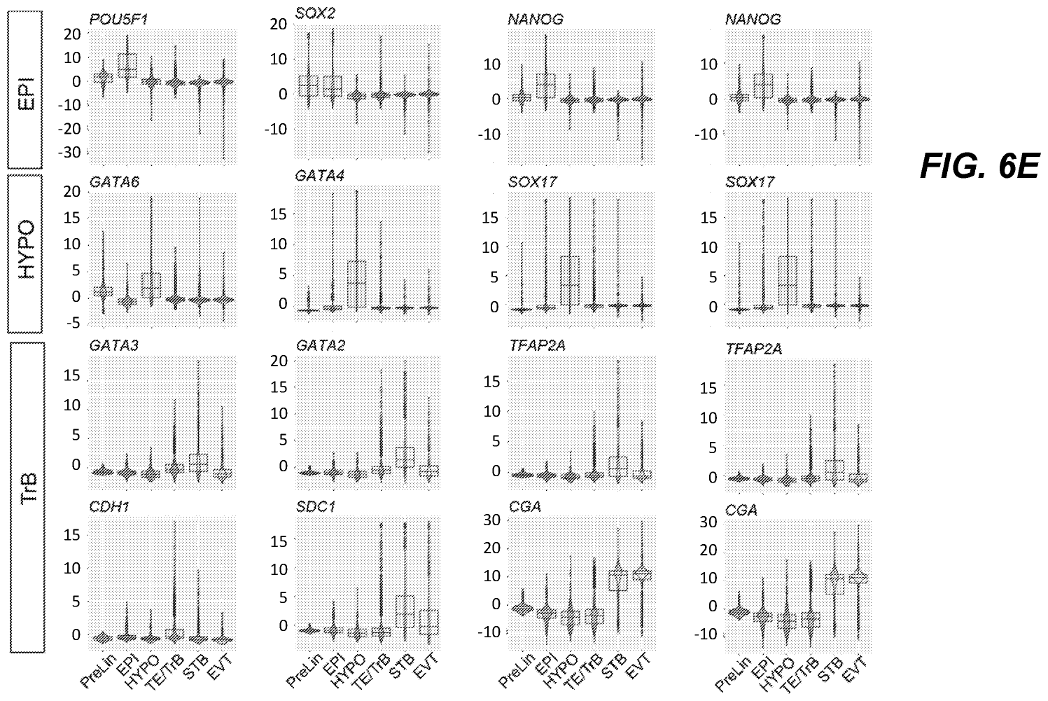

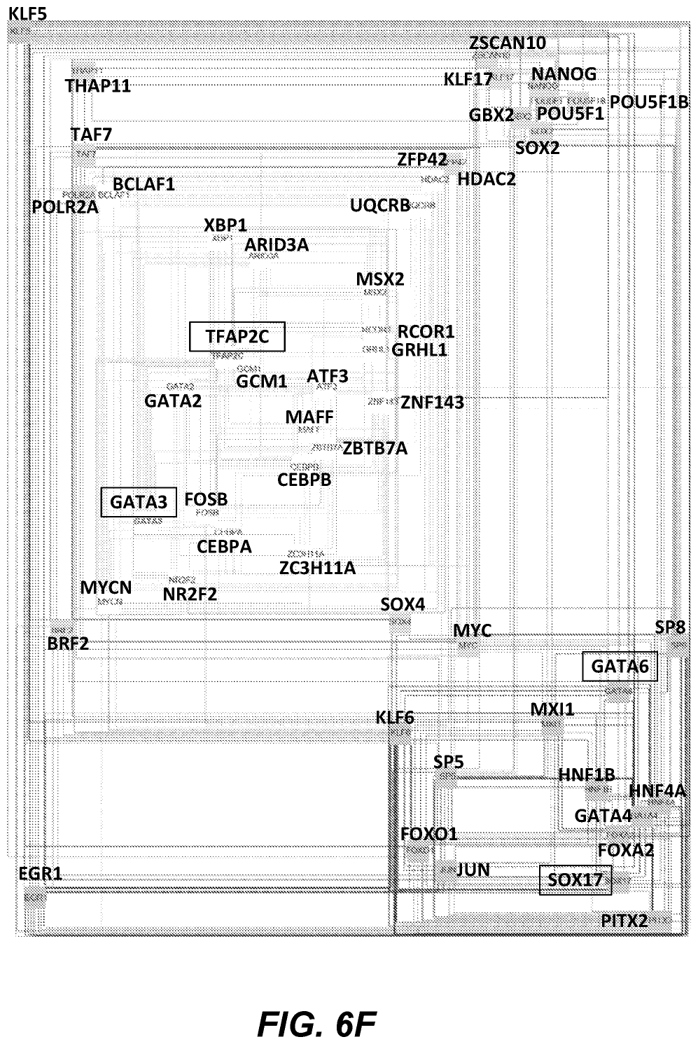

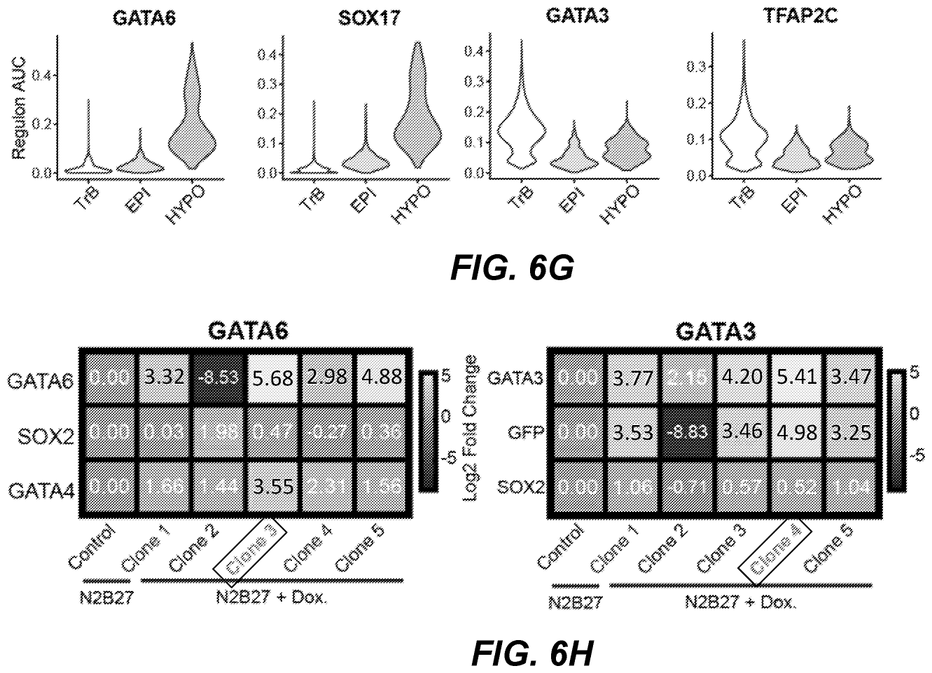

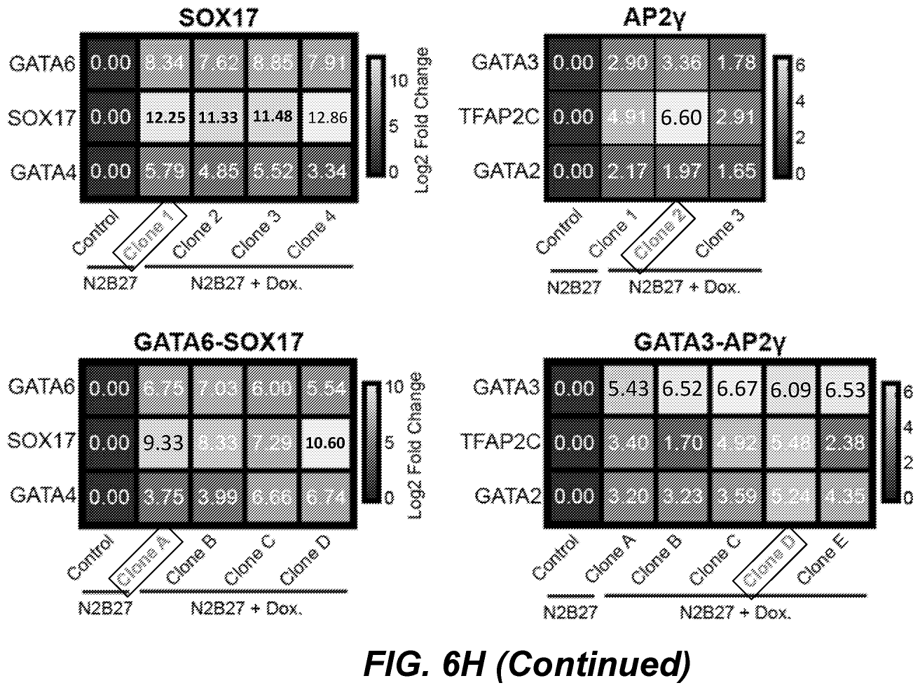

Factors that can similarly upregulate extraembryonic gene programs in human ESC was first identified. Published single cell RNA-sequencing data of human embryos cultured until gastrulation was integrated (FIG. 6A-FIG. 6E). The computational tool, SCENIC, was used to score predicted activity of transcription factors enriched in the epiblast, trophoblast or hypoblast (FIG. 6F). As expected, SOX2, NANOG, and POU5F1 (OCT4) showed high predicted activities in the epiblast. Transcription factors, including GATA4, GATA6, SOX17, and FOXA2, were particularly active in the hypoblast and GATA3, NR2F2, GATA2, and TFAP2C (AP2γ) showed enriched activities in the trophoblast (FIG. 6F-FIG. 6G). Overexpression of GATA6 or SOX17 has been shown to drive endodermal gene programs from primed hESCs. Therefore, GATA6 or SOX17 were selected as candidates to program hESCs to become hypoblast-like. Similarly, GATA3 and TFAP2C have been reported to share high chromatin co-occupancy during differentiation of hESCs into trophoblast stem cells. GATA3 and TFAP2C also demonstrated high predicted activity in trophoblast. Thus, GATA3 and TFAP2C were select as candidates to drive hESCs to become trophoblast-like. hESCs were generated and validated with doxycycline-inducible individual or combined transgenes for the transcription factors of interest (FIG. 1A-FIG. 1B and FIG. 6H).

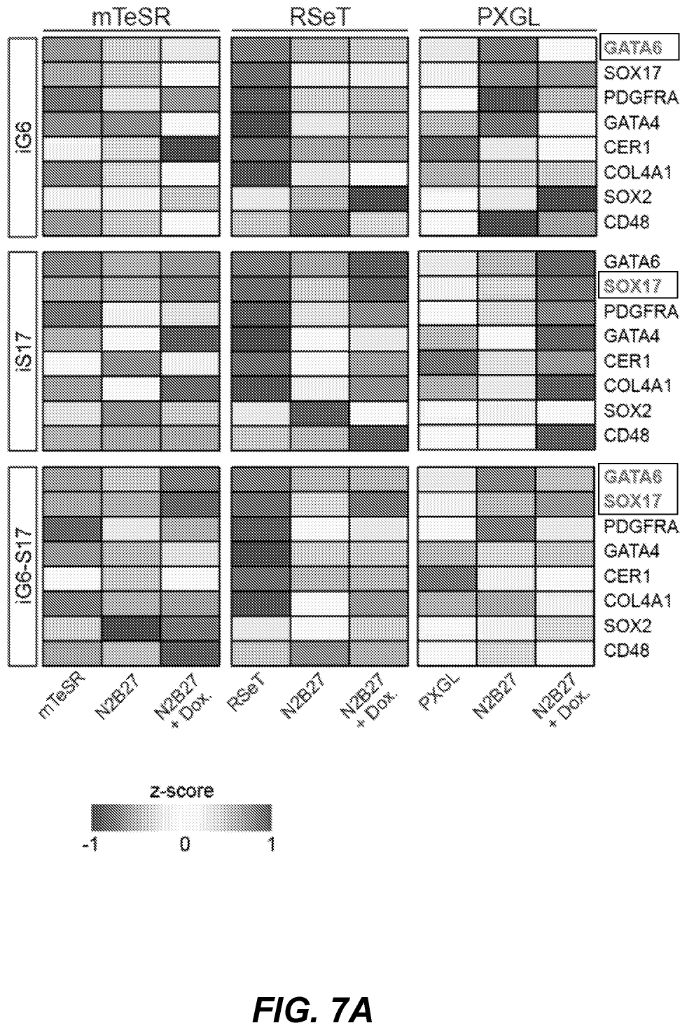

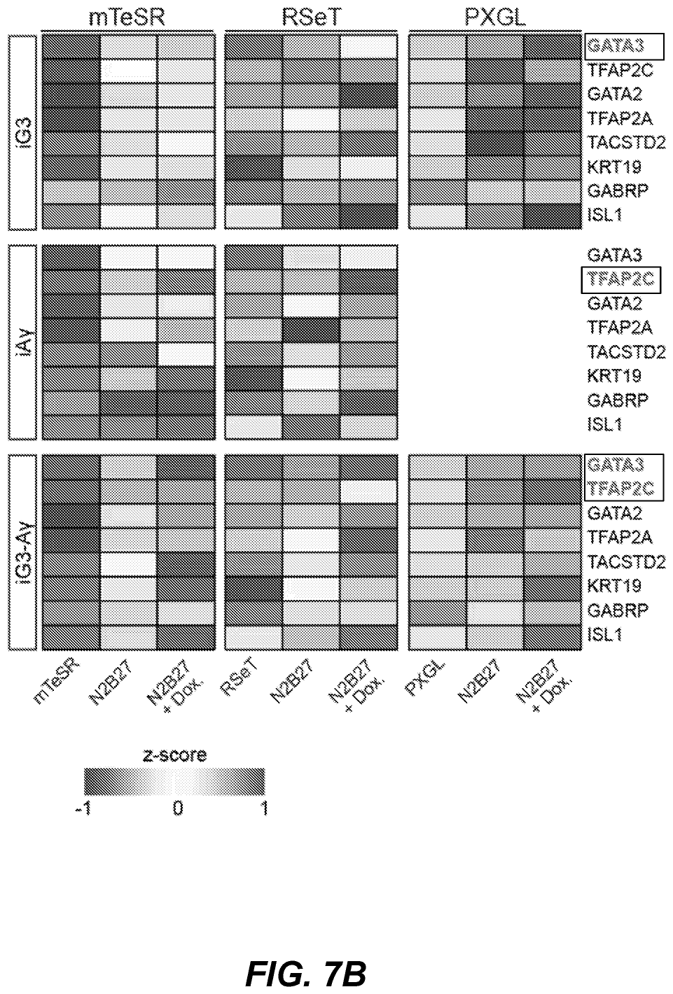

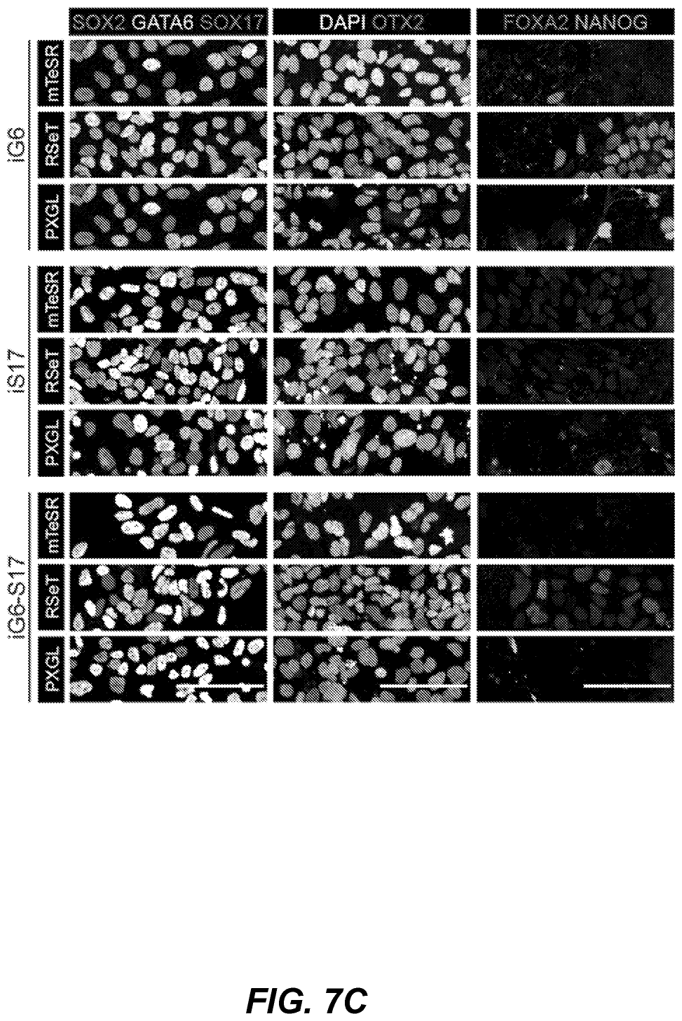

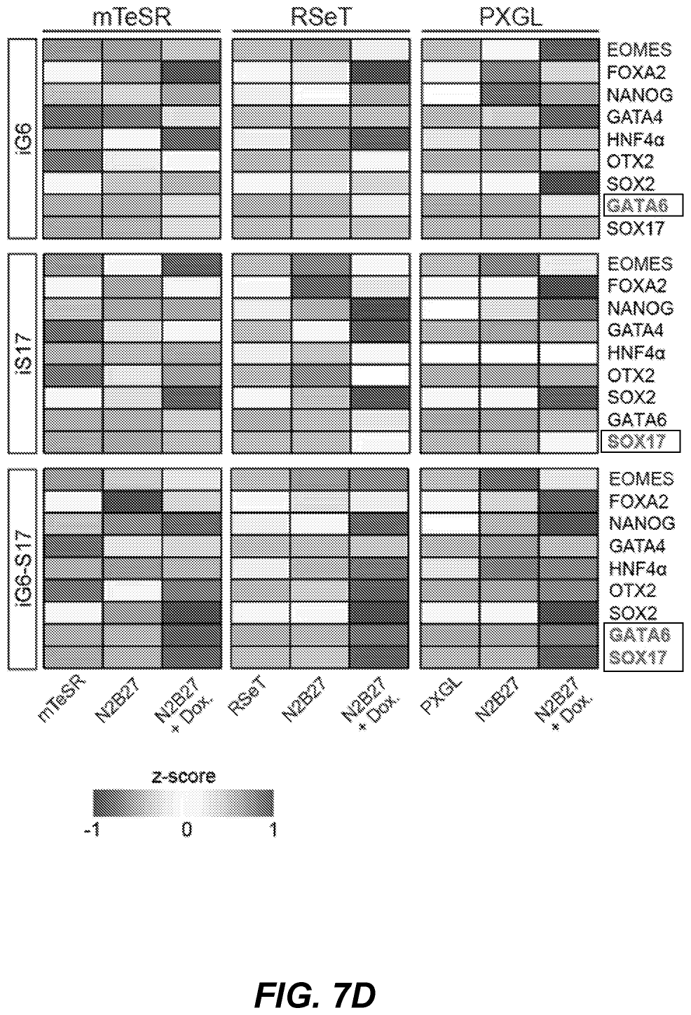

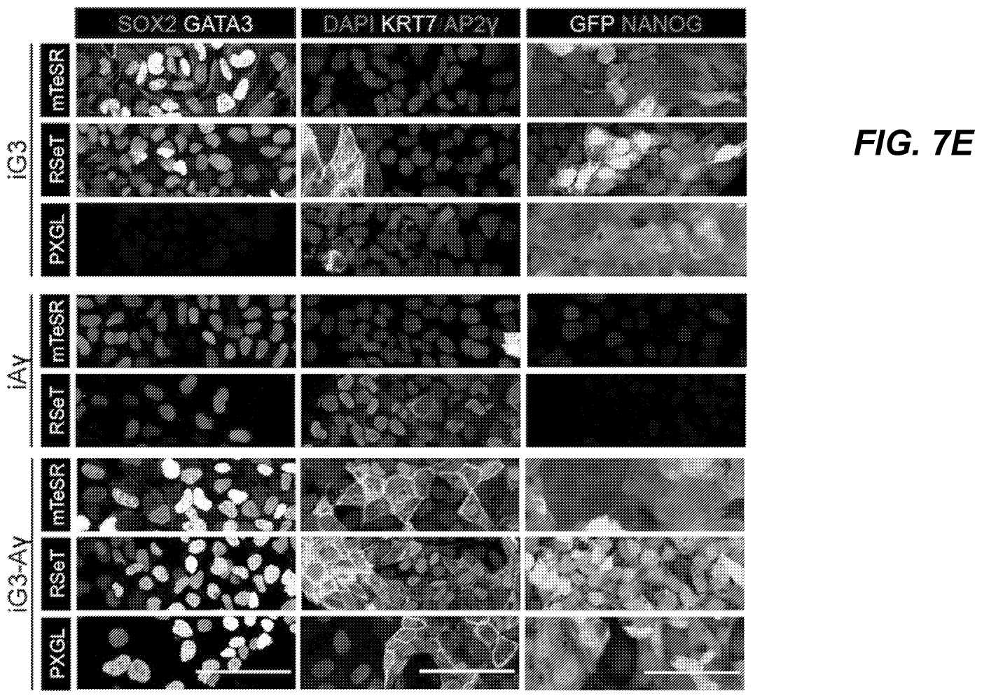

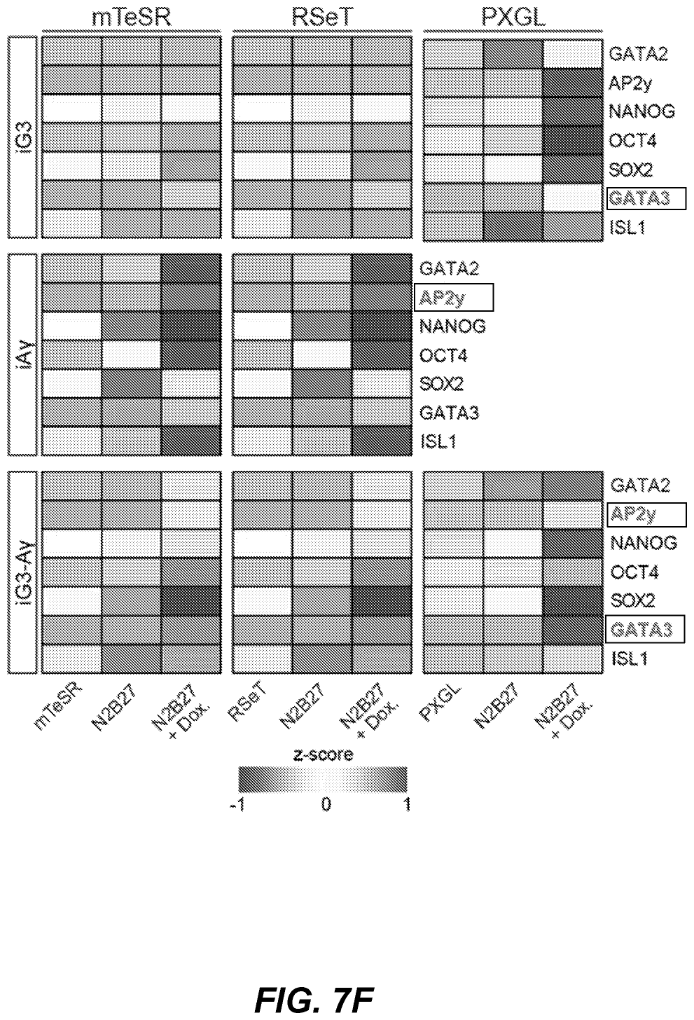

Notably, pluripotent state, in part, dictates differentiation potential from ESCs. Therefore, to assess the capacity of the selected candidate transcription factors to drive hESCs toward extraembryonic-like expression profiles, they were overexpressed—singly and in combination—in cells across the naïve-to-primed pluripotency spectrum. Cells were cultured using three established starting conditions: PXGL, which supports pre-implantation-like cells; RSeT, which generates intermediate peri-implantation-like cells; and conventional mTeSR1 conditions to maintain post-implantation-like cells. Significant differences were observed in extraembryonic gene induction using both individual or combined transgenes and starting from different pluripotency states, at both the protein and mRNA level (FIG. 7A-FIG. 7F). In hypoblast-like induction, GATA6 overexpression did not drive SOX17 expression from RSeT or PXGL conditions but SOX17 overexpression resulted in robust GATA6 upregulation across starting pluripotency state conditions (FIG. 7A, and FIG. 7C-FIG. 7D). FOXA2 expression was consistently upregulated after combined GATA6 and SOX17 induction from primed and RSeT, but not from PXGL conditions (FIG. 7C-FIG. 7D). These data indicated that while GATA6 and SOX17 could indeed drive endodermal gene programs, the regulation of specific downstream targets differed depending on the initial pluripotency state.

The AP2γ transgene appeared particularly effective in upregulating GATA2 and CK7 expression when driving trophoblast-like gene programs. However, induction of AP2γ alone resulted in cell death and loss of transgene expression in primed, but not RSeT or PXGL, cells (FIG. 7E-FIG. 7F). Combined induction of GATA6 and SOX17 or GATA3 and AP2γ resulted in consistent downregulation of pluripotency markers, including NANOG, SOX2, and OCT4 (FIG. 7A-FIG. 7F).

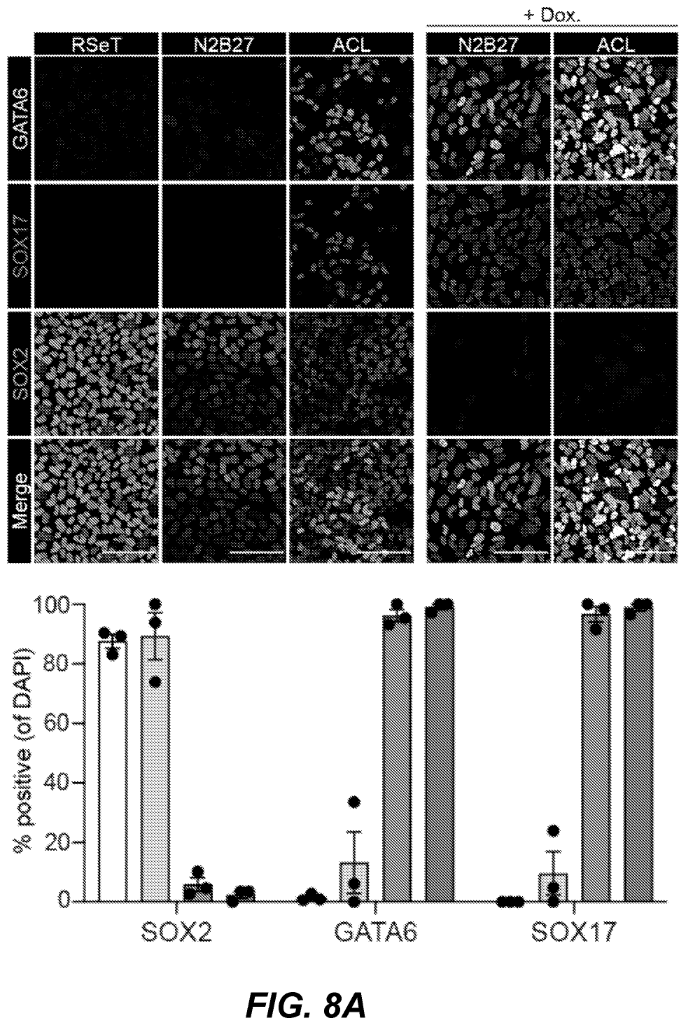

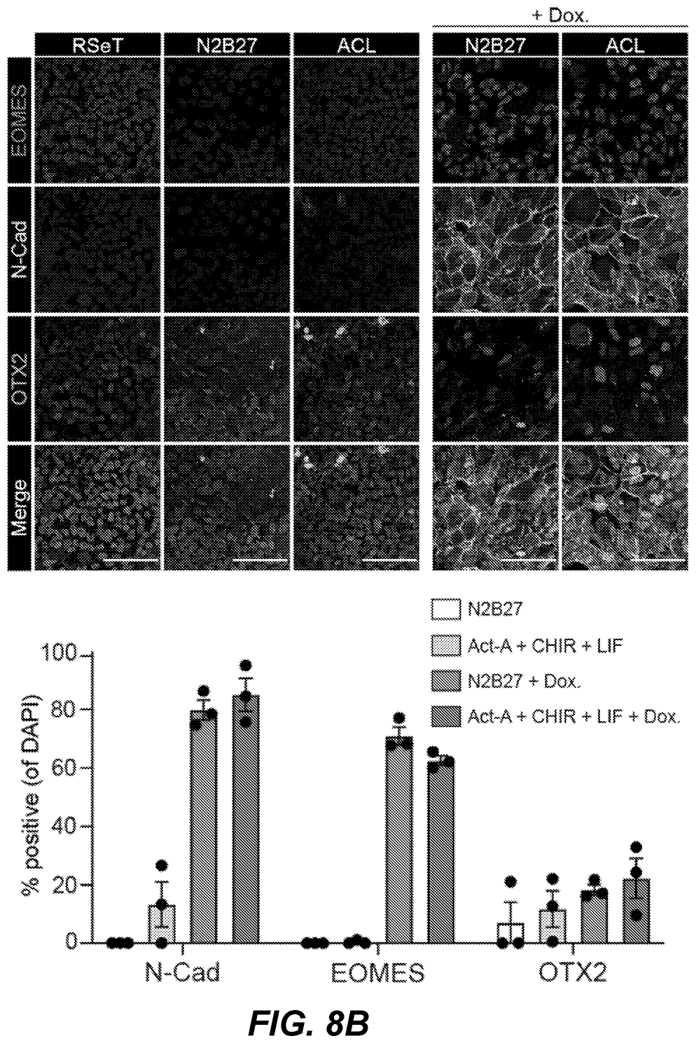

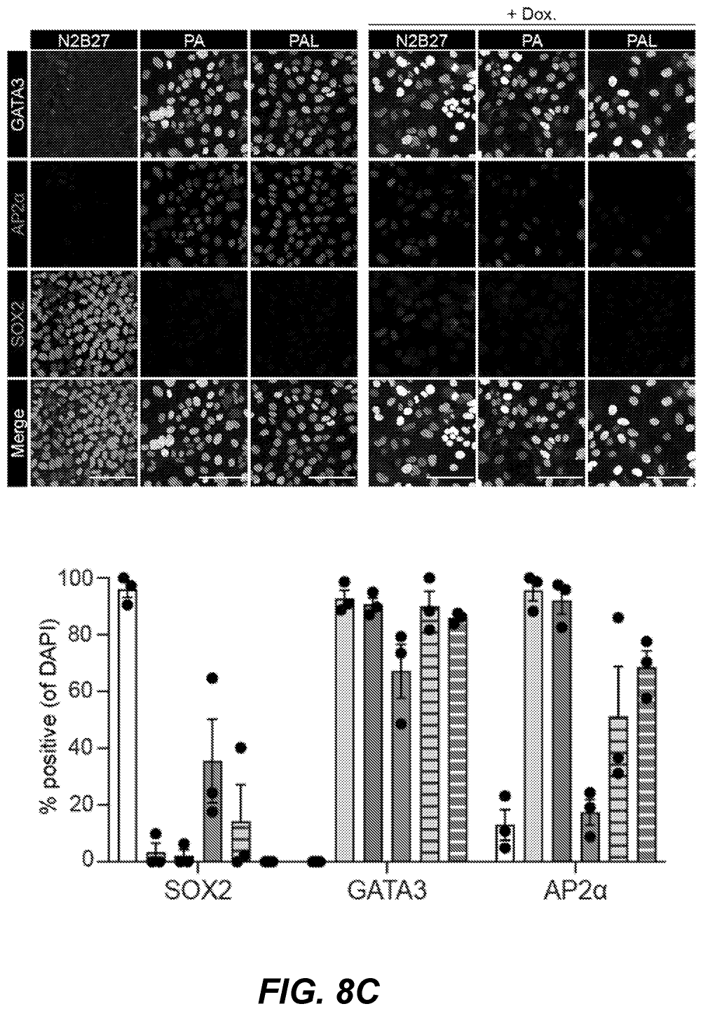

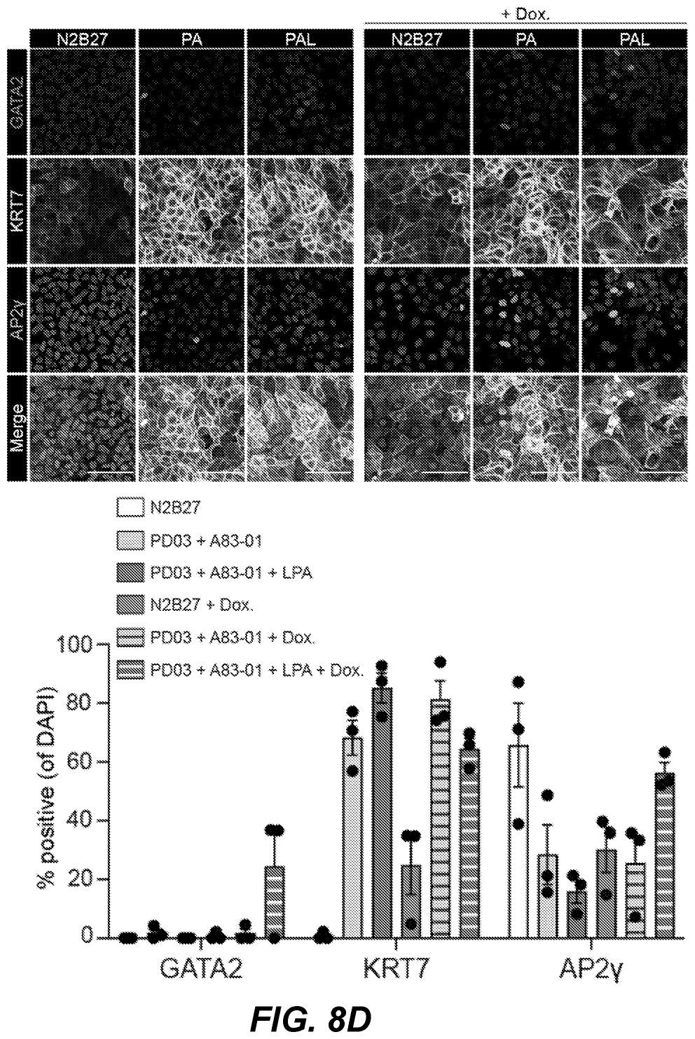

RSeT hESCs could be the best starting cell type to generate the presently disclosed model of the human post-implantation embryo because they: (1) represented a peri-implantation stage of development; (2) expressed low levels of amnion-specific genes after induction of GATA3 and AP2γ compared to primed cells (FIG. 7B, and FIG. 7E-FIG. 7F); and (3) are known to be more readily differentiate to peri- and post-implantation yolk sac-like endoderm cells as compared to PXGL cells. For these reasons, and the synergistic action of dual induction of candidate transcription factors, inducible GATA6-SOX17 and inducible GATA3-AP2γ RSeT hESCs were used for hypoblast-like and trophoblast-like cell induction, respectively, in subsequent experiments. Dual induction of GATA6 and SOX17 from RSeT cells in basal media induced endodermal gene expression equivalent to directed differentiation protocols in yolk sac-like cell differentiation conditions (FIG. 8A-FIG. 8B). Dual induction of GATA3 and AP2γ from RSeT cells in basal media induced trophoblast gene expression, albeit at varied levels when compared to directed trophoblast differentiation protocols (FIG. 8C-FIG. 8D).

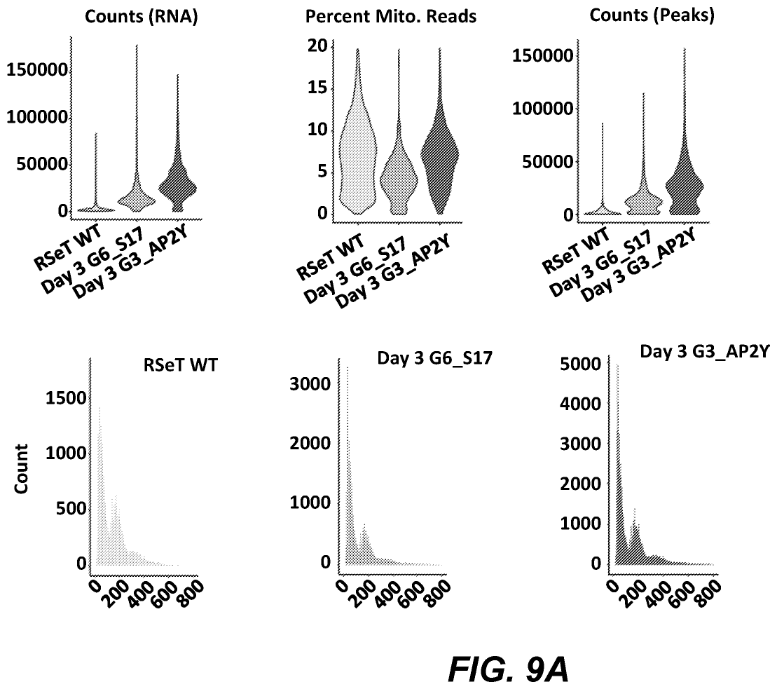

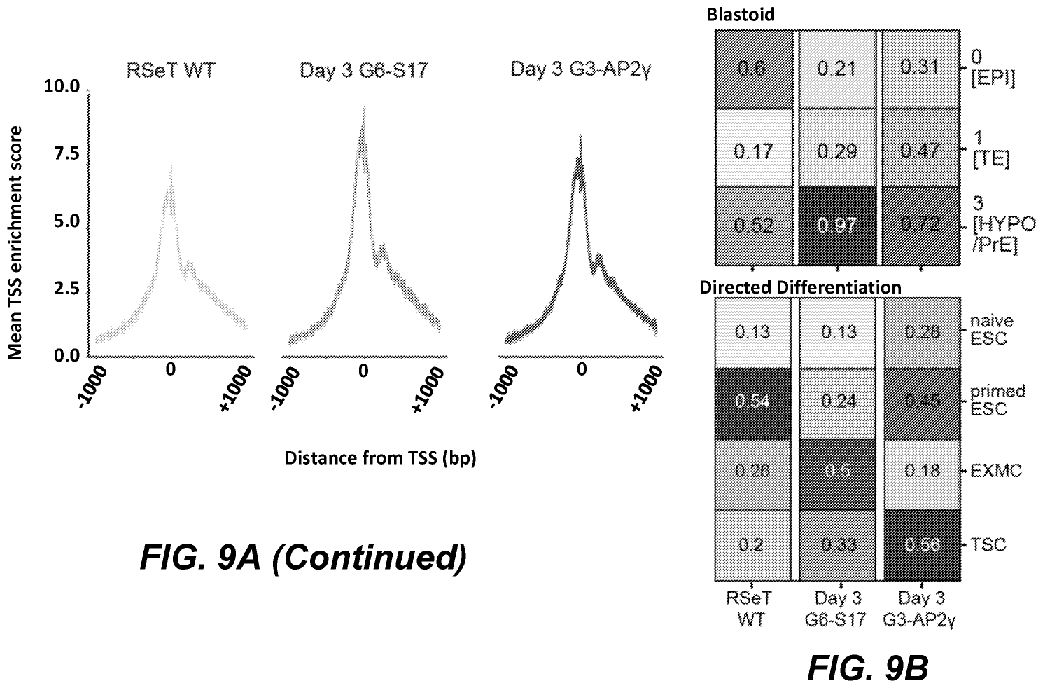

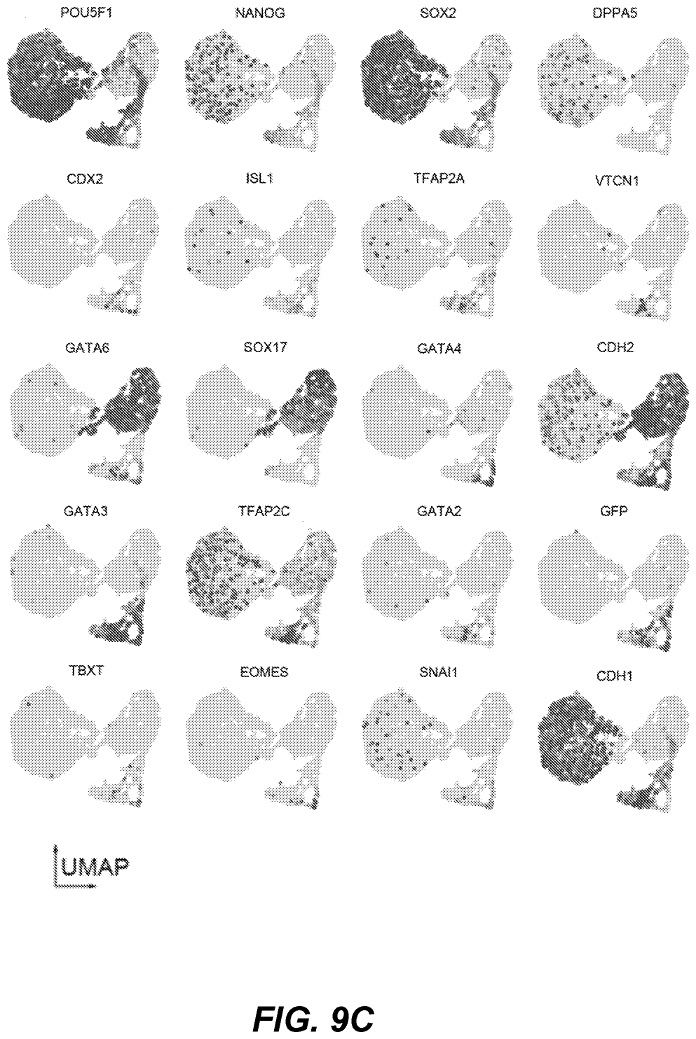

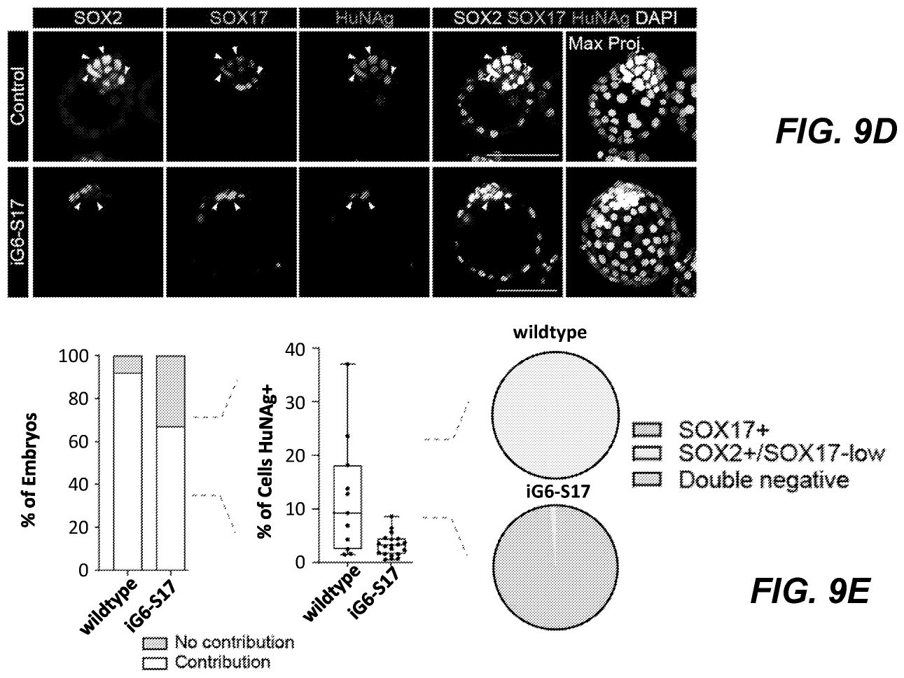

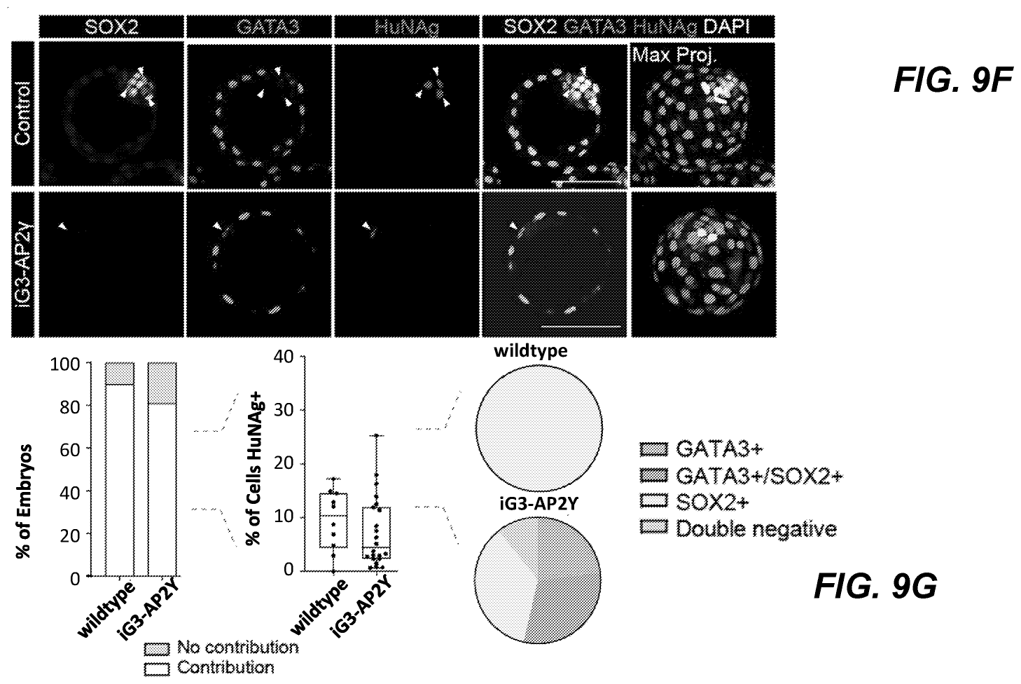

To further characterize RSeT hESCs induced to express GATA6-SOX17 or GATA3-AP2γ, single cell 10× multiome sequencing was carried out. Transcriptomic and chromatin accessibility were assessed simultaneously. Cells were clustered based on sample origin (FIG. 1C, and FIG. 9A). The application of a logistic regression framework showed that the wildtype, inducible GATA6-SOX17, and inducible GATA3-AP2γ RSeT hESCs had the highest similarities to the epiblast, hypoblast, and cytotrophoblast of the post-implantation embryo, respectively (FIG. 1D). Further, compared to in vitro blastoid and directed differentiation models, RSeT hESCs showed similarity to the pluripotent population, inducible GATA6-SOX17 cells were similar to blastoid-derived hypoblast, and inducible GATA3-AP2γ cells showed similarity to post-implantation-like trophoblast stem cells, but not blastoid-derived trophectoderm-like cells (FIG. 9B). Analysis of differentially expressed genes and differentially accessible motifs revealed similar embryonic and extraembryonic dynamics (FIG. 1E, and FIG. 9C). Specifically, enriched expression and motif accessibility scores of pluripotency and epiblast markers in RSeT hESCs, of hypoblast markers in GATA6-SOX17 inducible cells, and of trophoblast markers in the GATA3-AP2γ inducible cells were detected (FIG. 1E). Together, these data demonstrated that transcription factor-mediated induction of extraembryonic cell fate from RSeT hESCs drove hypoblast- or trophoblast-like gene programs without the need for exogenous factors, albeit heterogeneously and with some deficiencies in marker gene expression (FIG. 9C). The ability of inducible cells propagate in culture as stable cell lines was not tested. When aggregated with 8-cell stage mouse embryos, human cell contribution shifted towards SOX17-positive primitive endoderm and GATA3-positive trophectoderm for the inducible GATA6-SOX17 and GATA3-AP2γ cells, respectively, relative to wildtype controls (FIG. 9D-FIG. 9G). If this relative shift towards extraembryonic identity was sufficient to allow for self-organization, it would overcome the challenge presented for successful co-culture of embryonic and extraembryonic-like cells caused by their conflicting culture media requirements. Indeed, 1:1:1 co-culture of wildtype RSeT hESCs with inducible GATA6-SOX17 and inducible GATA3-AP2γ RSeT hESCs demonstrated good survival and mixed identity (FIG. 1F).

### Example 2

### Assembly of a 3D Post-Implantation Model

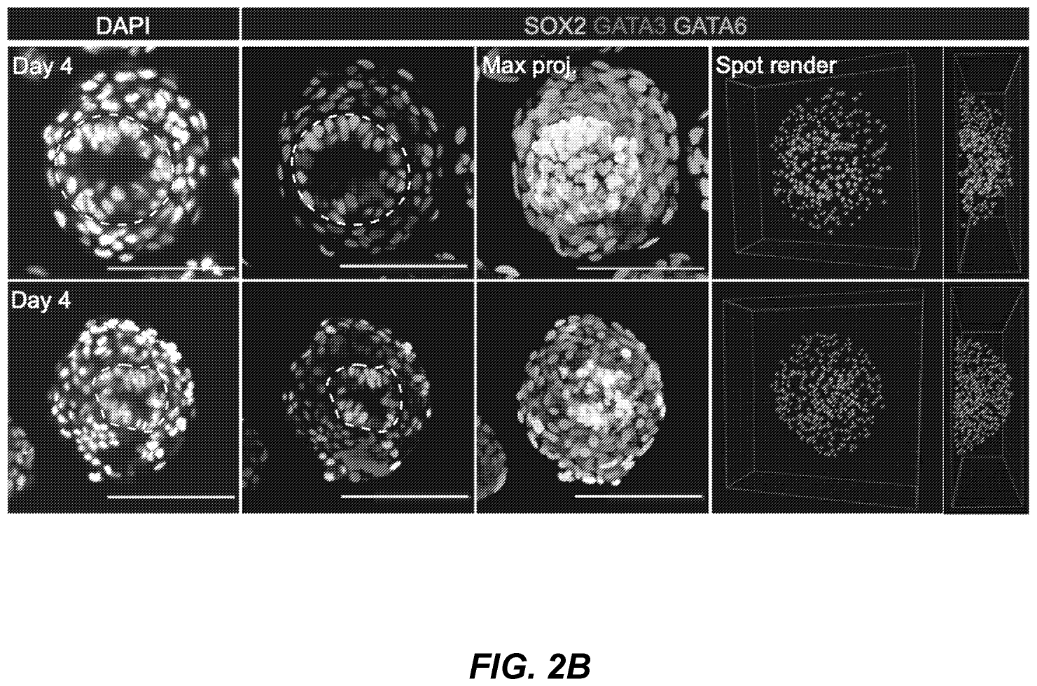

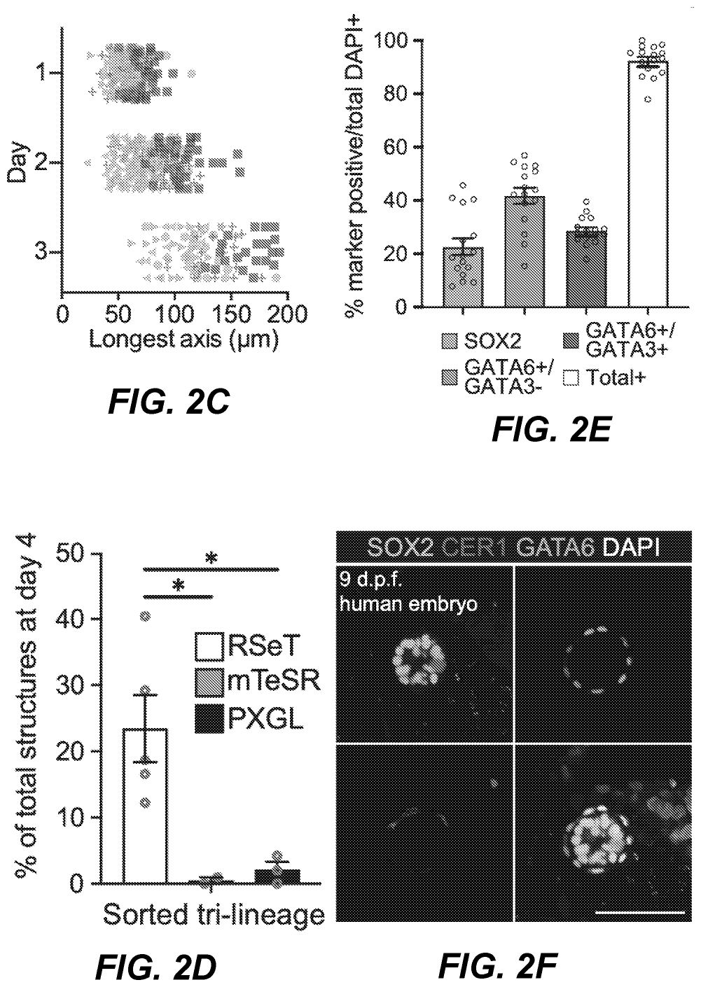

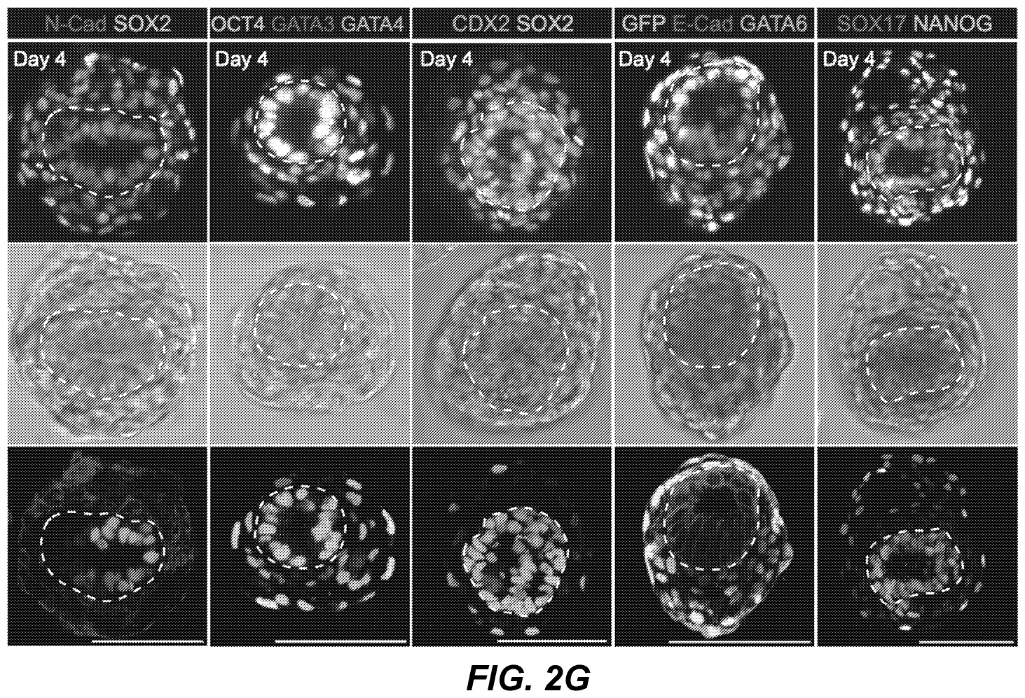

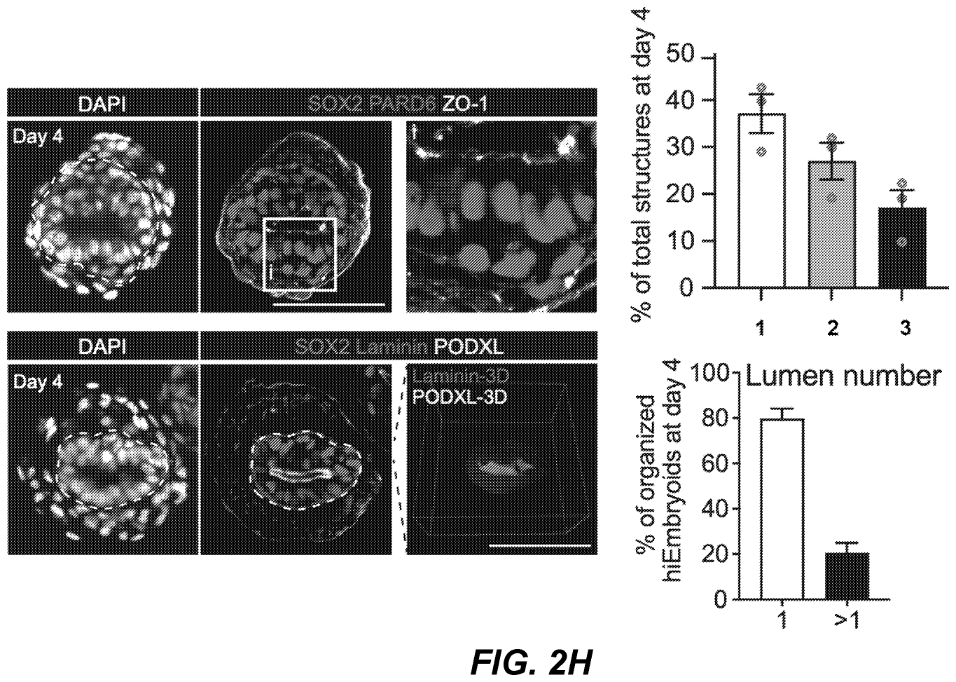

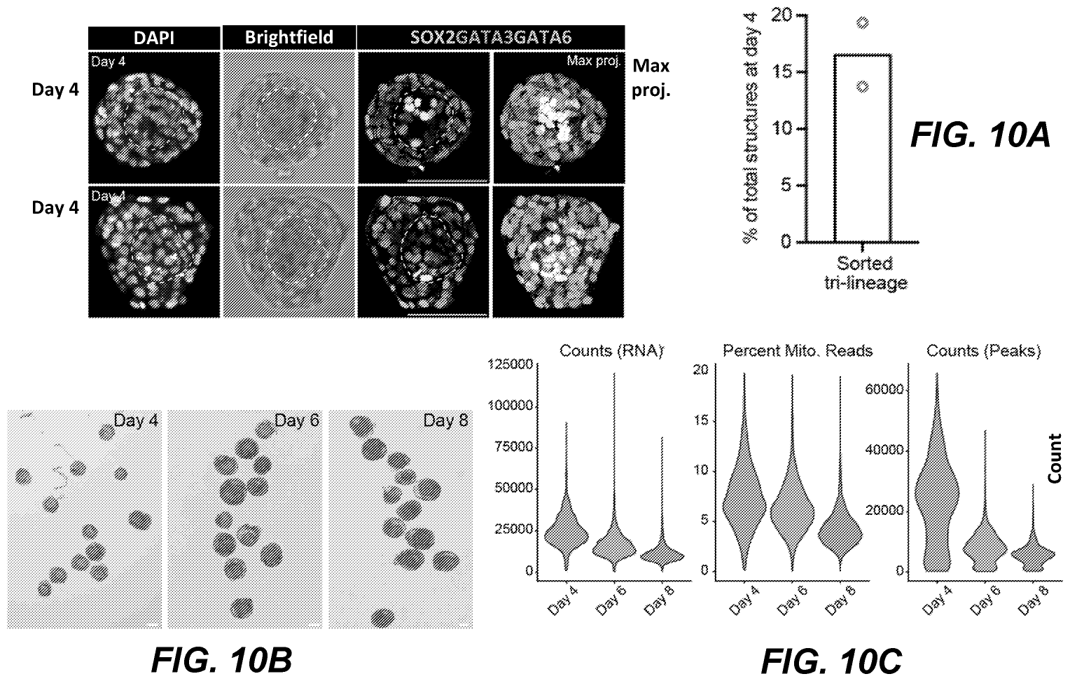

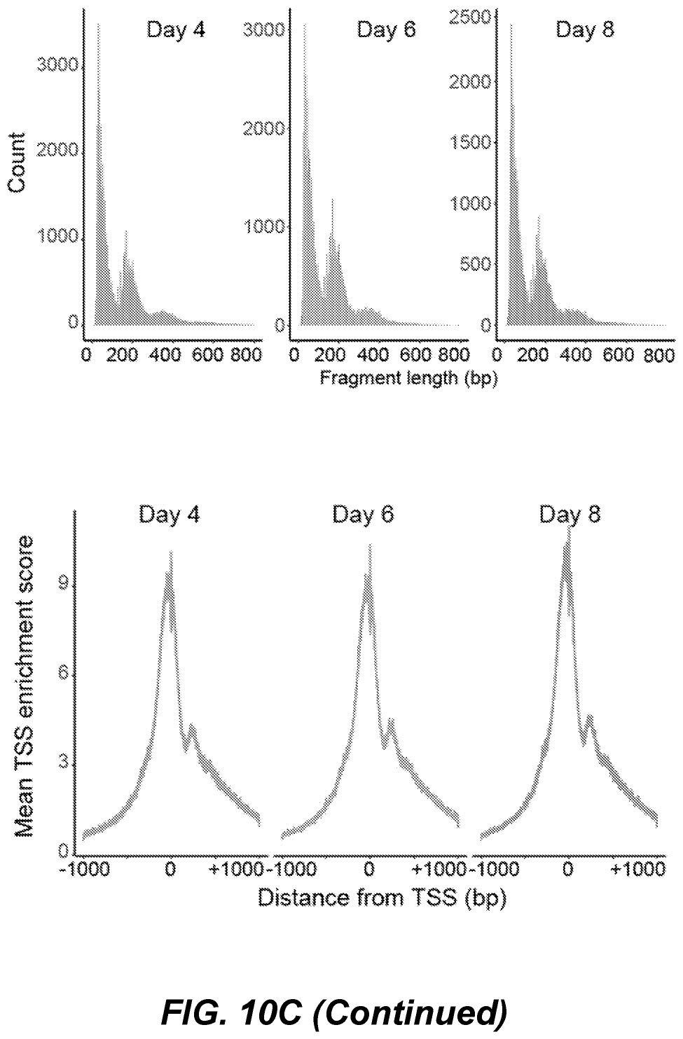

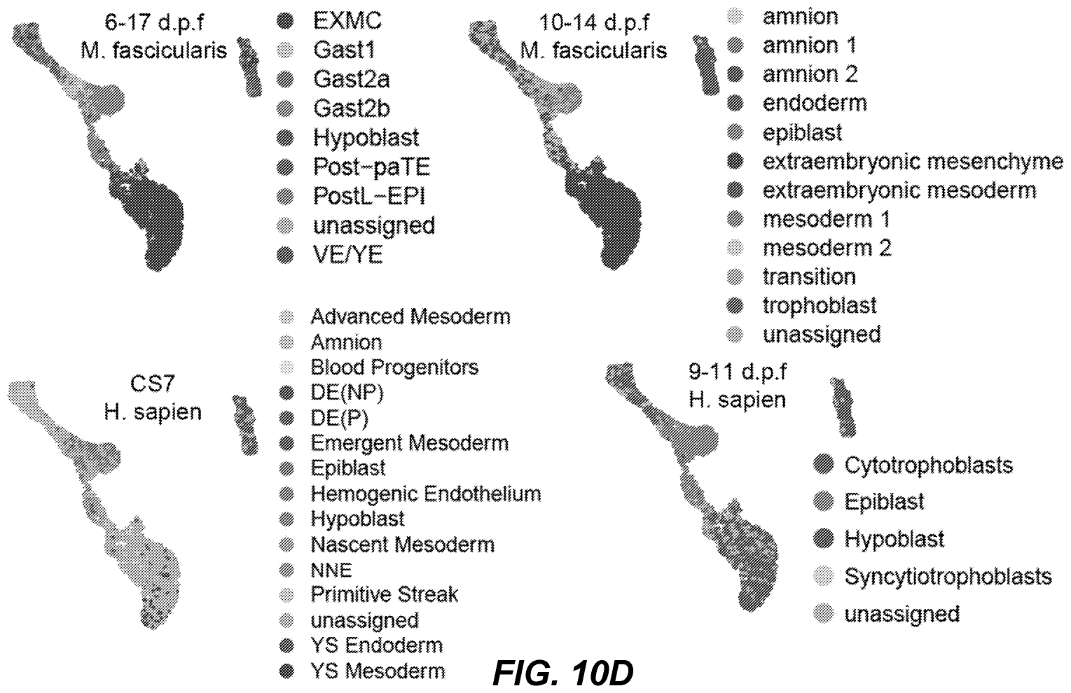

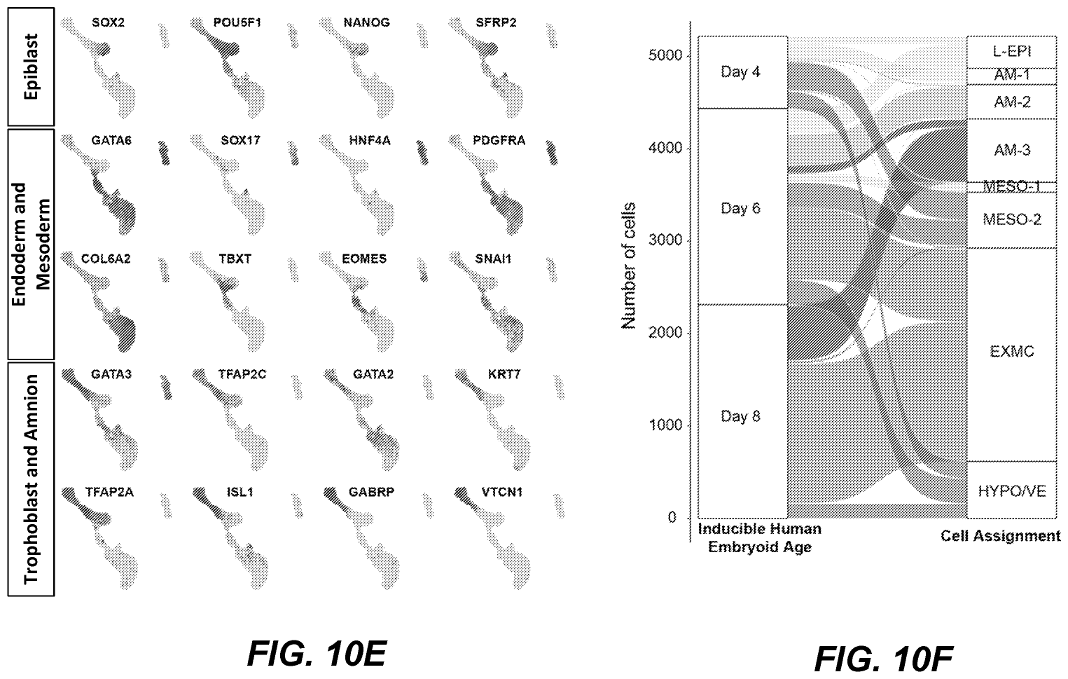

As all three RSeT hESC-derived cell types—wildtype, GATA6-SOX17 inducible, and GATA3-AP2γ inducible—can be cocultured in N2B27 medium, expression of the selected transcription factors was induced with doxycycline for 3 days. Subsequently, cell mixtures aggregated in Aggrewell dishes (FIG. 2A). Cells aggregated within 24 hours. By 48 hours post-aggregation, clear distinctions between inner and outer cellular domains were observed using brightfield (FIG. 2A). At 48 hours post-aggregation, the media was changed to post-implantation human embryo media (hIVC1). Incubation with doxycycline continued throughout the whole period of culture and proliferation was consistent across experiments (FIG. 2A and FIG. 2C). Four days post-aggregation, the cell aggregates had self-organized into structures with a SOX2-positive, epiblast-like domain containing a central lumen; an outer single layer of GATA3-positive putative trophoblast-like cells; and an intermediate putative hypoblast-like domain of GATA6-positive cells between inner lumenized domain and outer layer (FIG. 2B and FIG. 10A).

Similar to models of post-implantation mouse embryos, aggregates did not transit through a blastocyst-like morphology prior to forming post-implantation-like structures. The efficiency of inducible human embryoid formation (defined as aggregates containing an organized SOX2-positive domain, surrounded by concentric layers of GATA6-positive and GATA3-positive cells) was approximately 23% (FIG. 2D). In contrast, when using primed mTeSR1 or naïve PXGL hESCs as the starting pluripotency state for constitutive wildtype, GATA6-SOX17 inducible, and GATA3-AP2γ inducible cells, the efficiency of organized, multi-lineage structure formation was less than 5% (FIG. 2D-FIG. 2E). Organized embryo-like structures exhibited an organization reminiscent of the human embryo at 8-9 days post-fertilization (FIG. 2F).

The presently disclosed inducible human embryoids expressed several other lineage markers in an organized manner, including N-Cadherin, SOX17, and GATA4 in the putative hypoblast-like compartment (FIG. 2G). Structures with SOX17 and/or GATA6 expression were also observed within outer GATA3-AP2γ-induced cells (marked by eGFP), which may reflect the reported tendency of peripheral cells to adopt endodermal identities in embryoid bodies. The epiblast-like inner compartment expressed SOX2, NANOG, and E-Cadherin and maintained pluripotent and epithelial identity akin to the human embryo (FIG. 2G). Furthermore, this inner domain exhibited apicobasal polarity with basal deposition of laminin and apical expression of PODLX, PARD6, and ZO-1 (FIG. 2H). These data demonstrated that RSeT hESC-derived embryo-like structures can self-organize in minimal media conditions.

### Example 3

### Differentiation within Embryoids

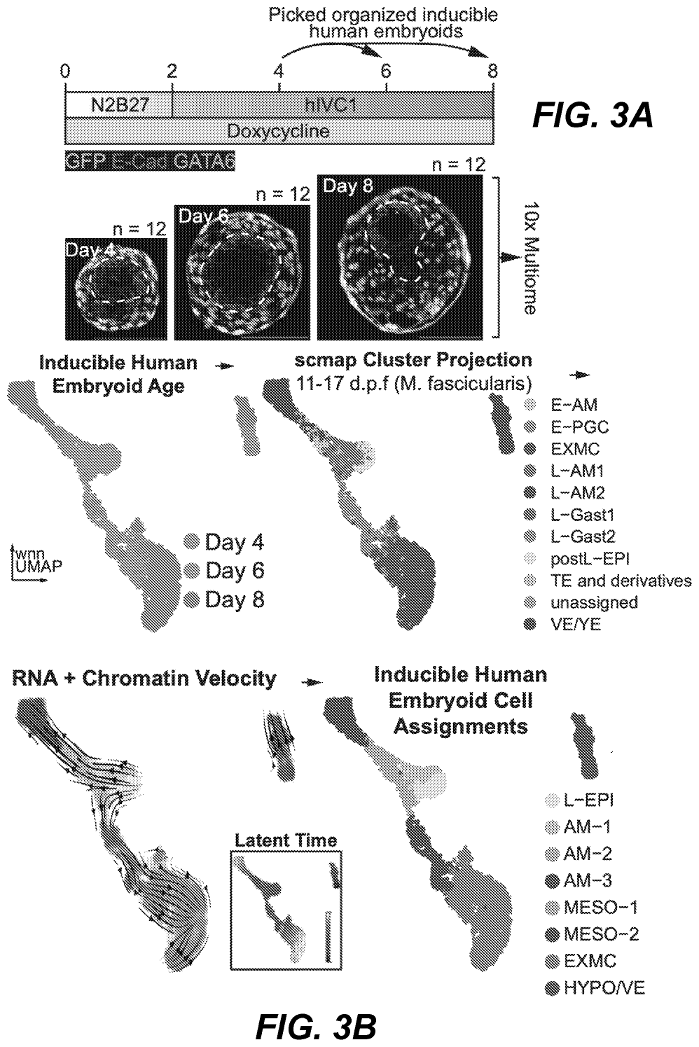

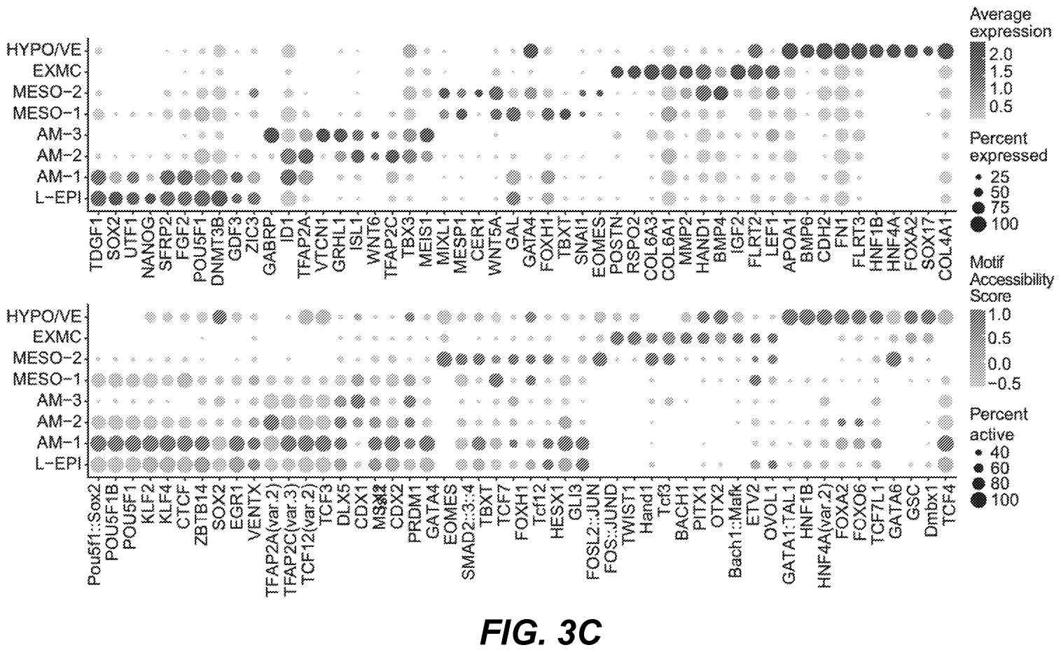

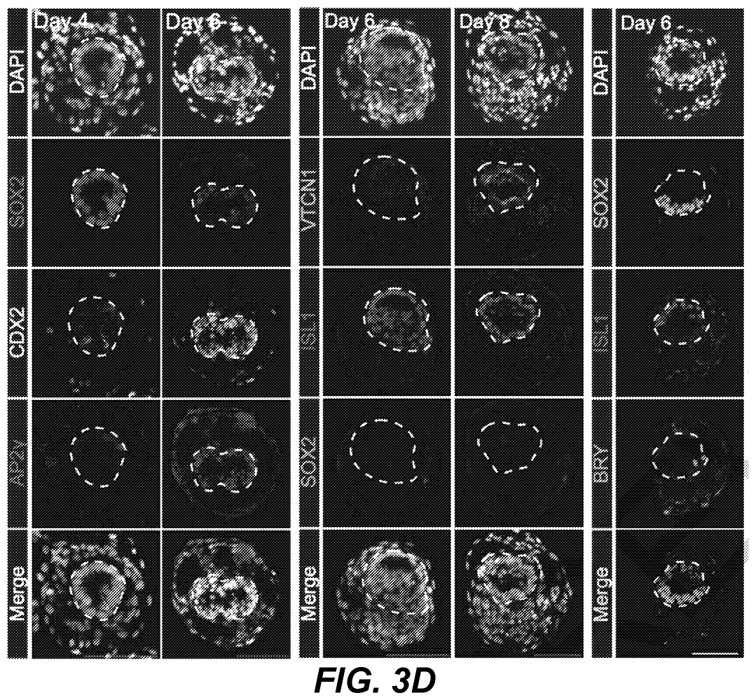

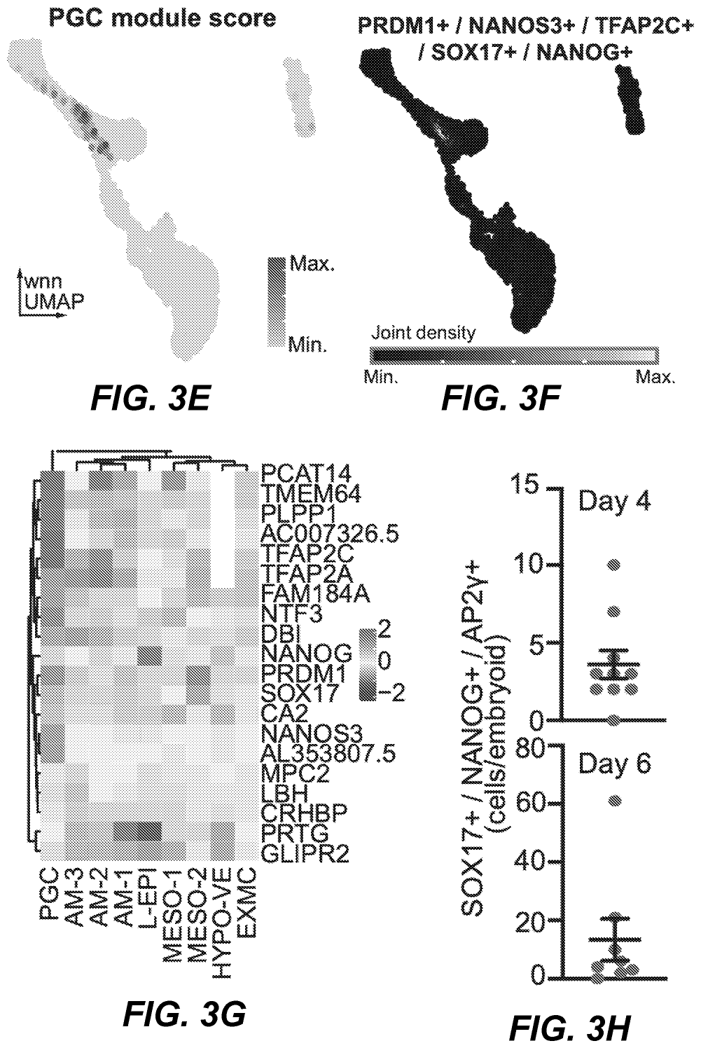

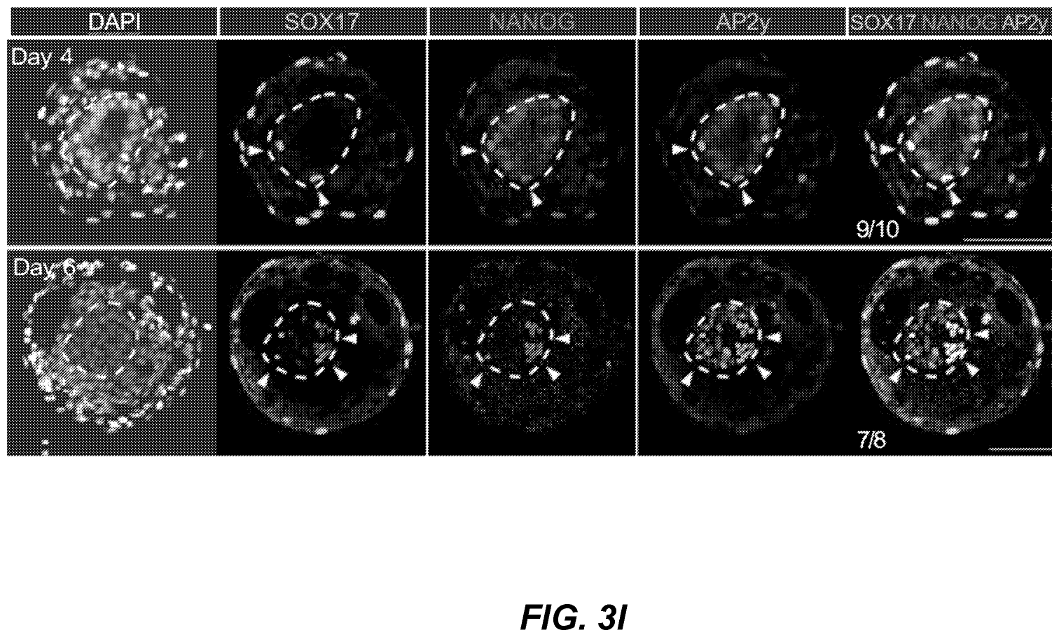

To gain insight into whether the presently disclosed human embryo-like model developed gene expression and chromatin accessibility patterns that reflected the natural human embryo, single cell multiome RNA and assay for transposase-accessibie chromatin with sequencing (ATAC-seq) at 4, 6 and 8 dpf were performed (FIG. 3A). Individual structures were selected for sequencing based on their development of the three tissues: (1) an inner, epithelial domain; (2) an intermediate domain surrounding the central epithelium; and (3) an outer GFP-positive cell layer (FIG. 10B-FIG. 10C). To assign clusters without bias, scmap was used to project dataset obtained in the present study onto human and cynomolgus macaque datasets spanning peri-implantation to gastrula stages (FIG. 3B and FIG. 10D). This analysis allowed one to project gene expression signatures from previously annotated cynomolgus macaque cell type clusters onto the presently disclosed model of the human embryo. Using multiome-based velocity inference (multivelo), it was found that the inferred differentiation time correlated well with the transition of structures from Day 4 to Day 8 post-aggregation. These data, in combination with canonical marker expression, allowed annotation of the cell types in the presently disclosed human embryo-like structures (FIG. 3B, and FIG. 10E-FIG. 10F). Clusters resembling embryonic late-epiblast (L-EPI), amnion (AM-1; AM-2; and AM-3), mesoderm (MESO-1; MESO-2), extraembryonic mesenchyme (EXMC), and hypoblast/visceral endoderm (HYPO/VE) were identified (FIG. 3C and FIG. 10E-FIG. 10F). These assigned clusters showed differences in their composition based on the day of sample collection, with the L-EPI cluster comprised of only Day 4 and Day 6 structures, a progressive shift from AM-1 to AM-2 and AM-3 over time, and similarly from mesoderm to EXMC (FIG. 10F).

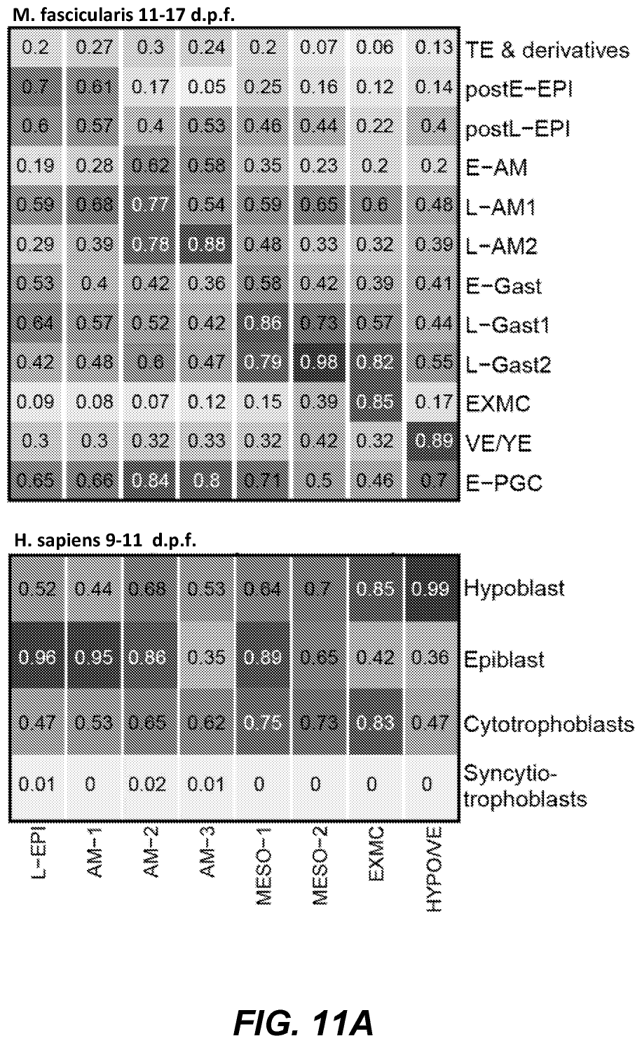

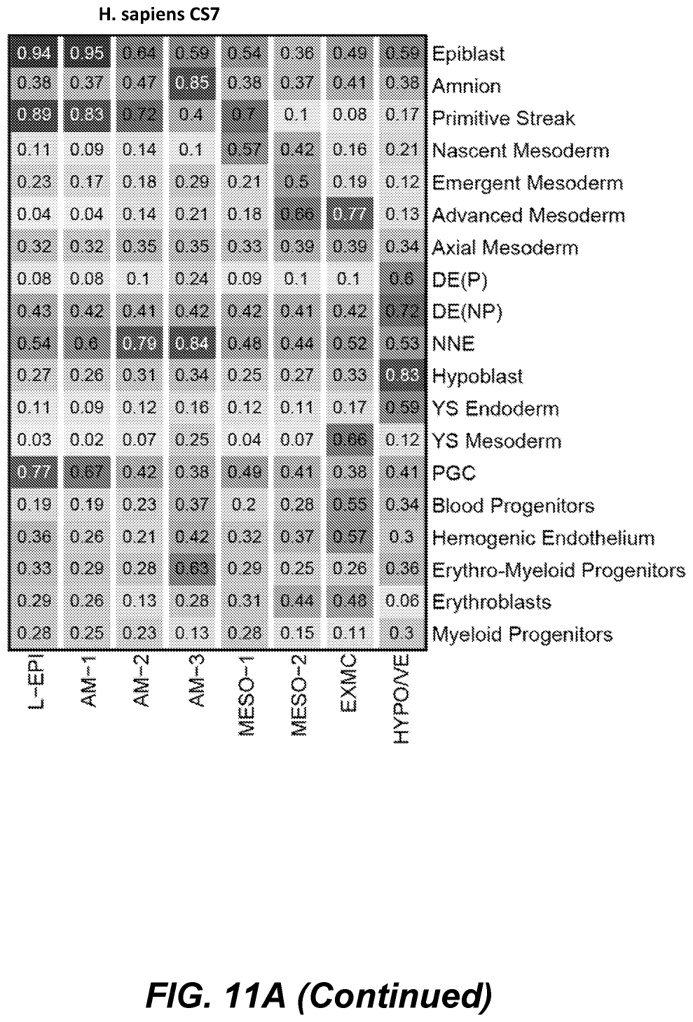

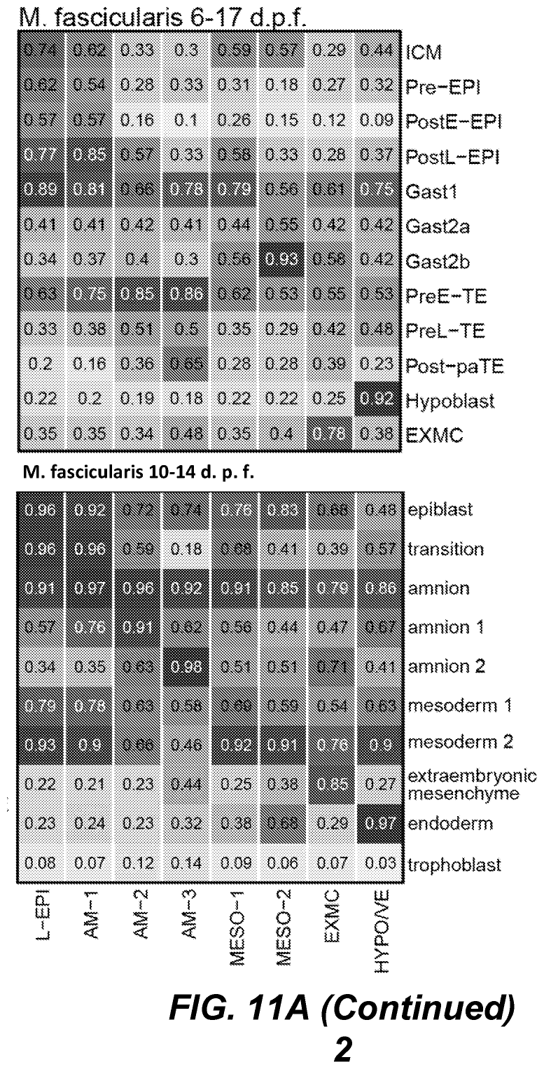

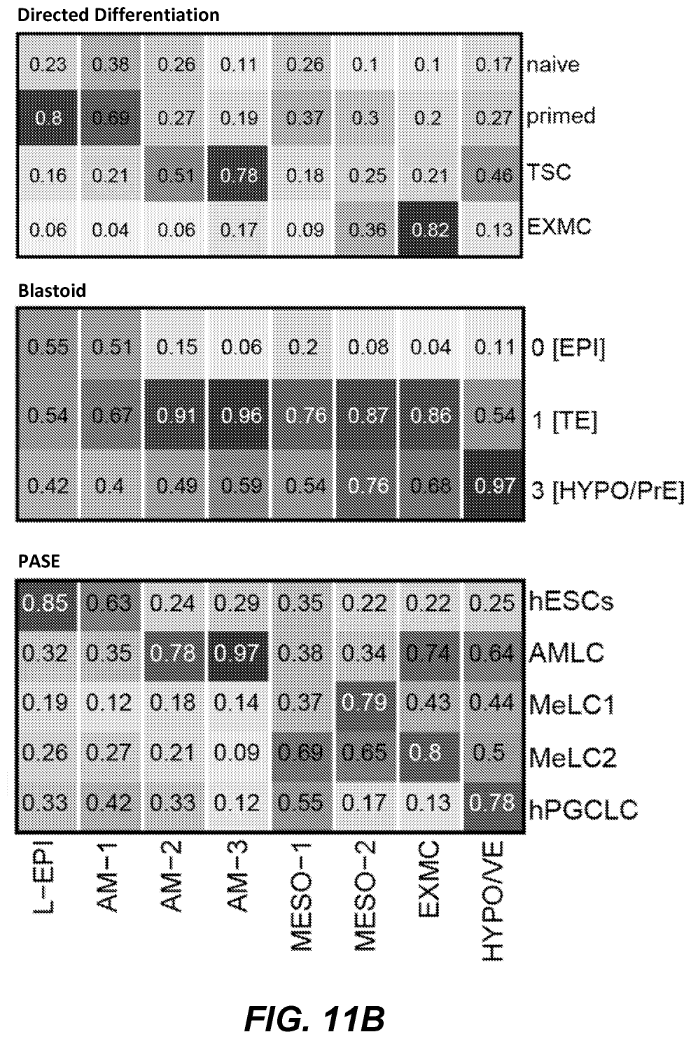

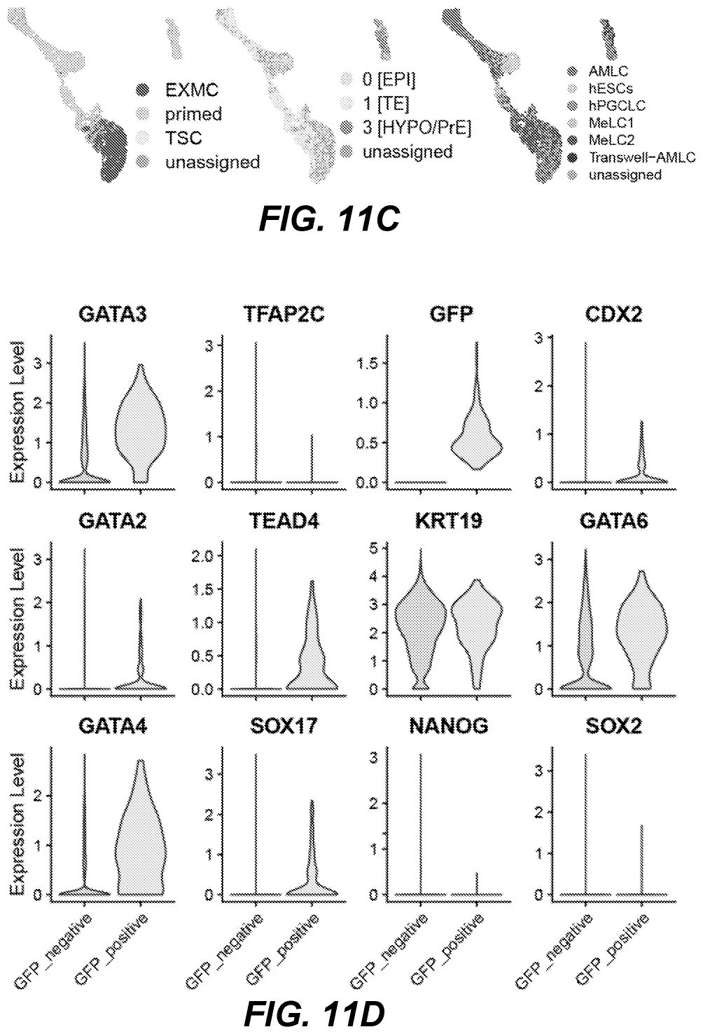

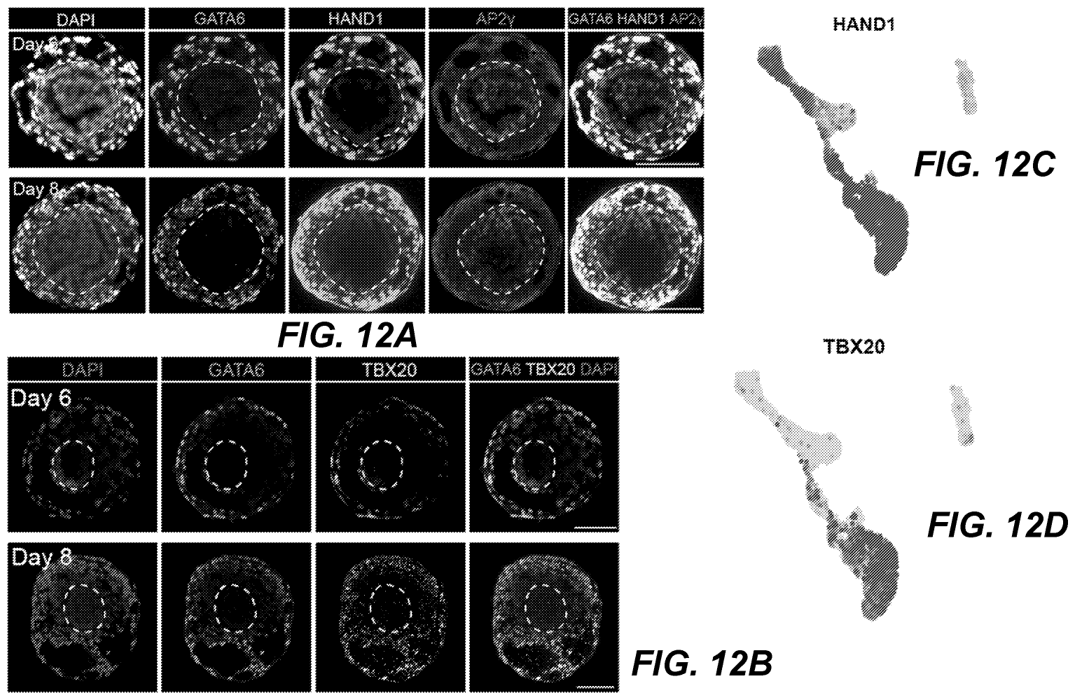

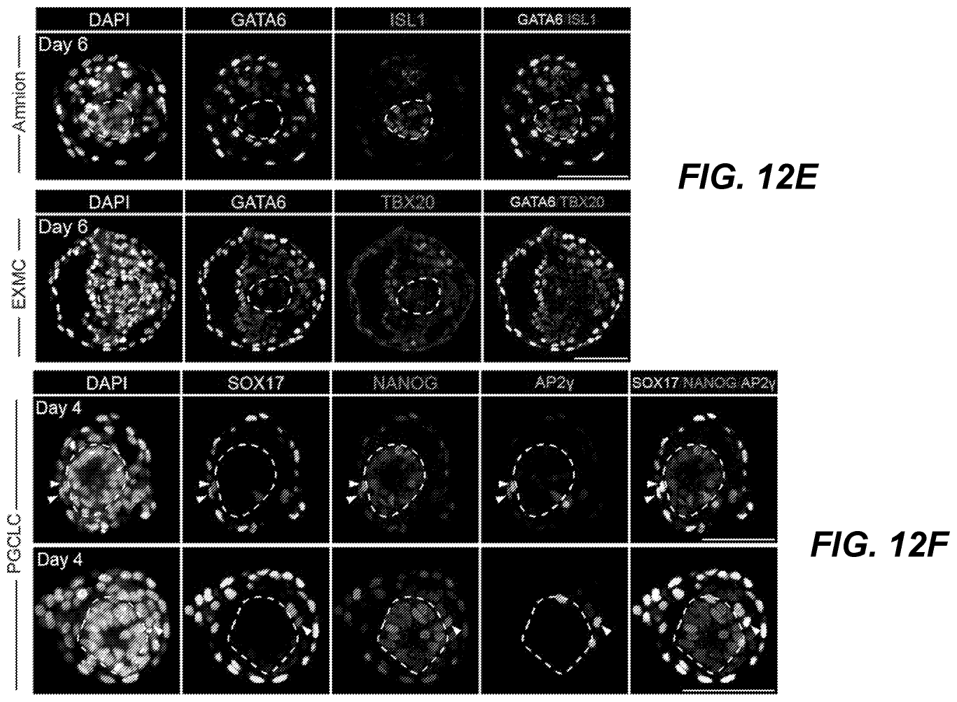

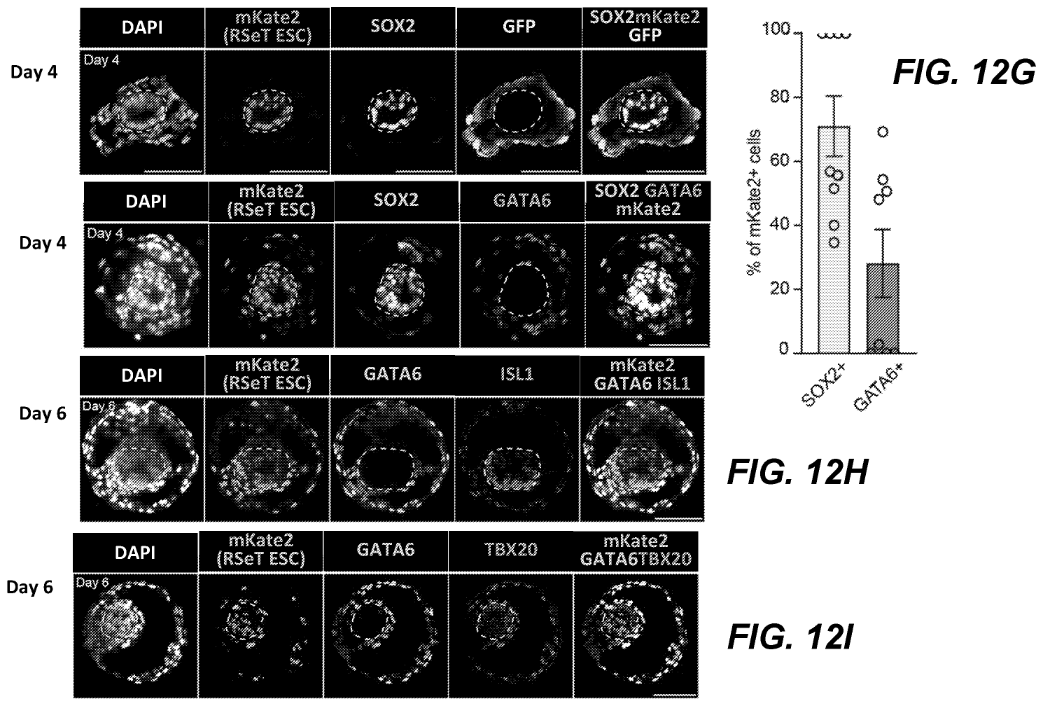

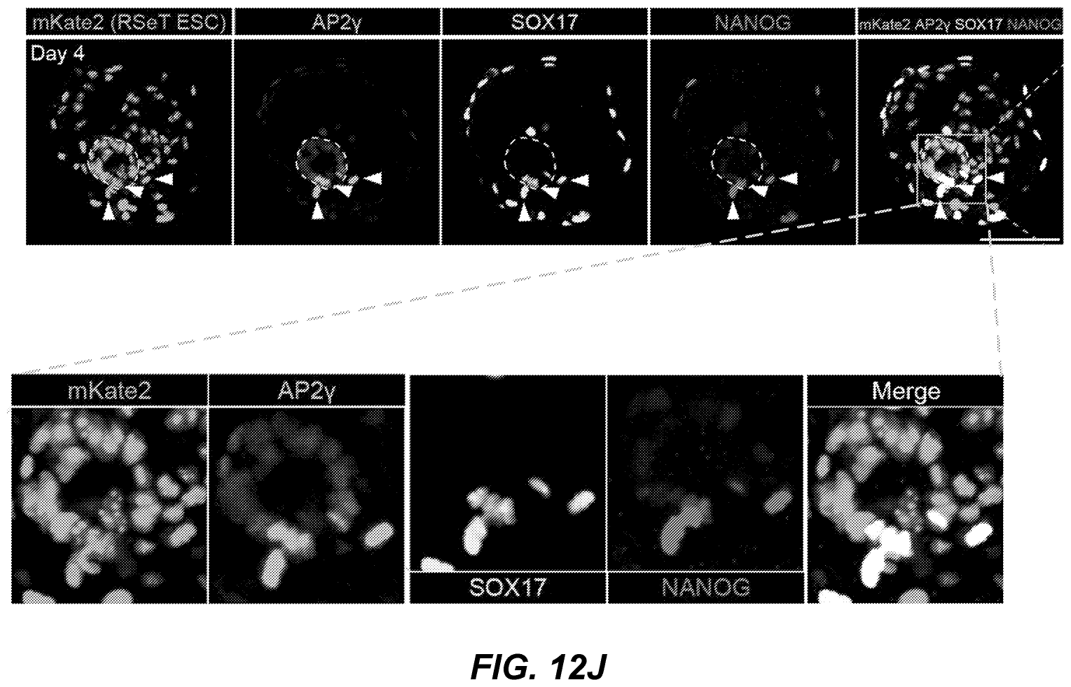

Lastly, the clusters described hereby were directly compared with previously annotated peri-implantation and peri-gastrulation cynomolgus macaque and human embryos datasets. This analysis demonstrated similarity between inducible human embryoid clusters and primate embryos (FIG. 11A-FIG. 11B). Similarly, inducible human embryoid clusters aligned with in vitro hESC-derived models including post-implantation amniotic sac embryoids (PASE), blastoids, and recently identified extraembryonic mesenchyme-like cells generated during TSC-like directed differentiation (FIG. 11B-FIG. 11C). Significant similarities between in vitro amnion, hypoblast, and extraembryonic mesenchyme populations were observed in comparison to embryo-like structures. However, a distinct trophoblast-like cluster derived from the GFP-positive inducible GATA3-AP2γ cells was not identified, despite its presence as an outer layer within the inducible human embryoids (FIG. 3A and FIG. 11D). Given the aberrant upregulation of endodermal markers after aggregation, inducible GATA3-AP2γ-derived cells were not likely to represent bona fide trophoblast. Nevertheless, inducible human embryoids generated several cell types, which failed to robustly differentiate, when human embryos were cultured in vitro to post-implantation stages, including amnion and extraembryonic mesenchyme. Indeed, immunofluorescence analysis demonstrated that the inner, SOX2-positive domain, upregulated amnion markers, including CDX2 and ISL1 by day 6 post-aggregation. By Day 8 post-aggregation, the inner domain expressed mature amnion markers VTCN1 and HAND1 (FIG. 3D and FIG. 11A-FIG. 11B) correlating to the transition between AM-1, AM-2, and AM-3. The GATA6-positive domain also expressed HAND1, supporting the presence of extraembryonic mesenchyme (FIG. 12A-FIG. 12B). A subset of GATA6-positive cells showed high co-expression of TBX20, further highlighting the presence of extraembryonic mesenchyme in this intermediate region (FIG. 12C-FIG. 12D). In the majority of the presently disclosed human embryoids, the entire epiblast-like domain differentiated toward an amnion fate. However, rare cases of embryo-like structures on Days 6-8 post-aggregation exhibited dorsoventral and/or anterior-posterior symmetry breaking with regionalized ISL1, SOX2, and BRACHYURY expression (FIG. 3D).

Recent reports have postulated that both amnion and primordial germ cells, the precursors to gametes, are at least partially generated from a bipotent progenitor. Therefore, whether such progenitors or their progeny were specified in the presently disclosed human embryo-like model was evaluated. A primordial germ cell module score was first assigned based on expression of genes identified in human primordial germ cell-like cells differentiated in vitro. Cells with transcriptomes resembling primordial germ cell-like cells were identified (FIG. 3E-FIG. 3F). Cells in the 98th percentile of primordial germ cell-like cell gene expression module scores were labeled as putative primordial germ cell-like cells (“PGC”). Primordial germ cell-like cells expressed TFAP2A (AP2a), a crucial marker of bipotent amnion and primordial germ cell-like cell progenitors. In contrast to other cells in AM-1 and AM-2 clusters, primordial germ cell-like cells expressed the pluripotency marker NANOG and primordial germ cell markers PRDM1 (also known as BLIMP1) and NANOS3 (FIG. 3G). Immunofluorescence analysis of a canonical set of human primordial germ cell markers4l confirmed that AP2γ/SOX17/NANOG triple-positive primordial germ cell-like cells were observed by 4 days post-aggregation and increased in number by Day 6 (FIG. 3H-FIG. 3I and FIG. 12F). These data demonstrated that robust primordial germ cell-like cell specification was concomitant with amnion-like cell formation and occurred within the inner epiblast-like compartment, supporting the existence of a bipotent progenitor for these two lineages.

Shef6-mKate wildtype ESCs were next utilized to confirm the differentiation trajectories within embryoids. On Day 4 post-aggregation, the majority of the wildtype cells contributed to the inner SOX2-positive epiblast-like domain, with some contribution to the GATA6-positive population (FIG. 12G), in line with a small proportion of early extraembryonic mesenchyme differentiation in sequencing analysis (FIG. 10F). By Day 6 post-aggregation, wildtype cells contributed to ISL1-positive amnion-like domain, as well as GATA6 and TBX20-positive extraembryonic mesenchyme-like cells (FIG. 12H and FIG. 12I). Lastly, the derivation of AP2γ/SOX17/NANOG triple-positive primordial germ cell-like cells from the mKate2 labeled wildtype population was confirmed (FIG. 12J). These data confirmed that the epiblast-like domain differentiated toward several post-implantation lineages.

### Example 4

### BMP Mediates Epiblast Differentiation

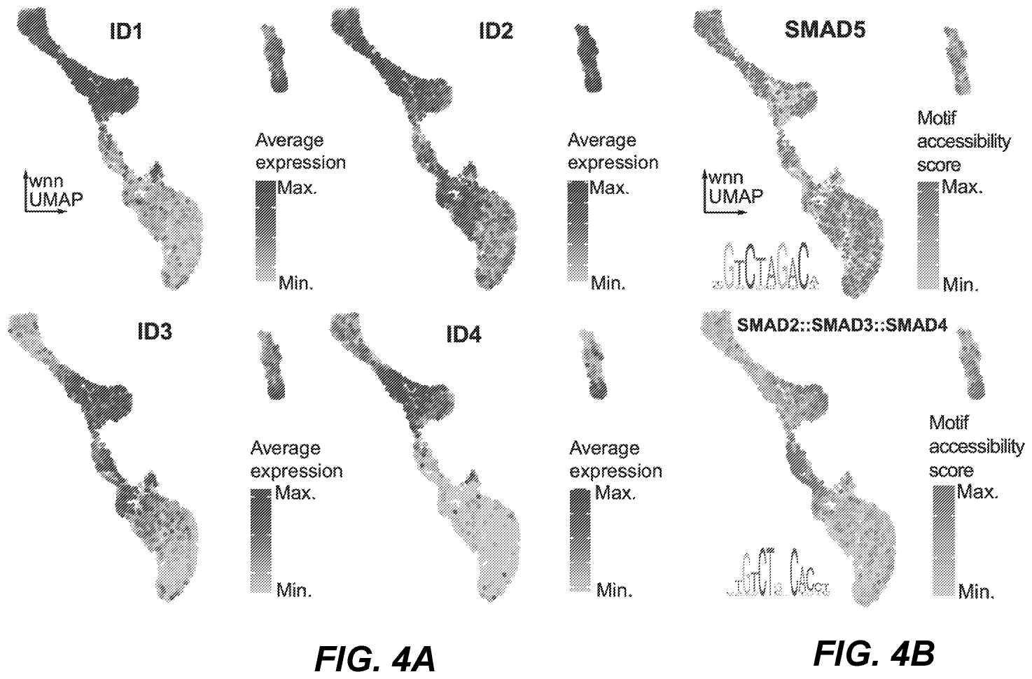

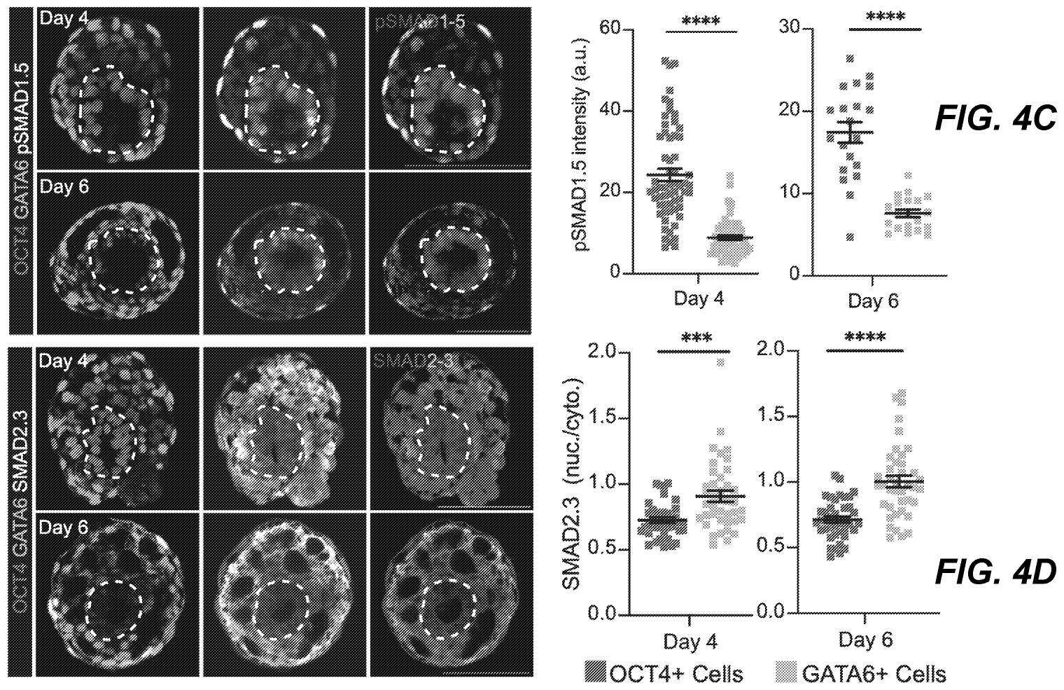

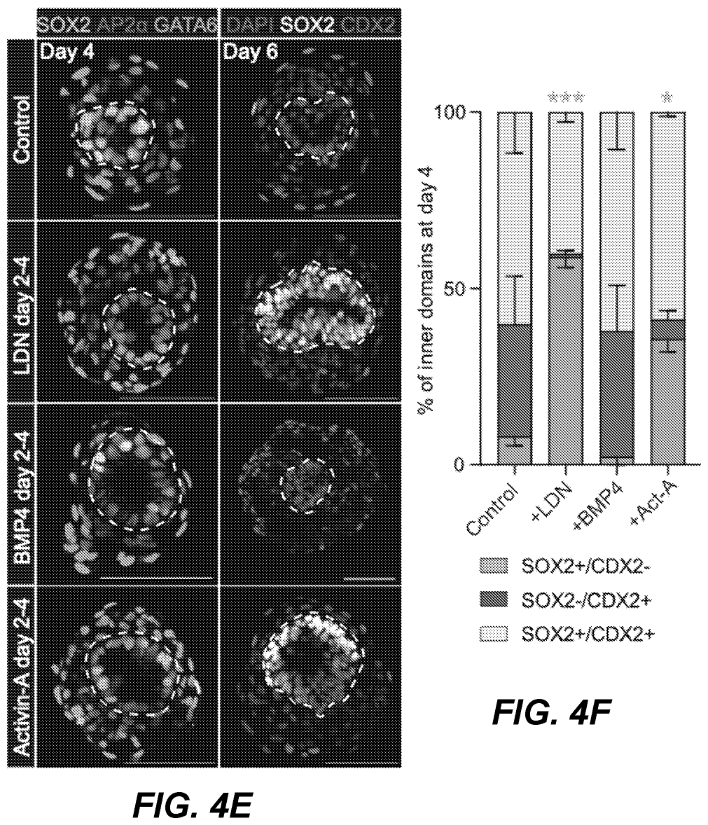

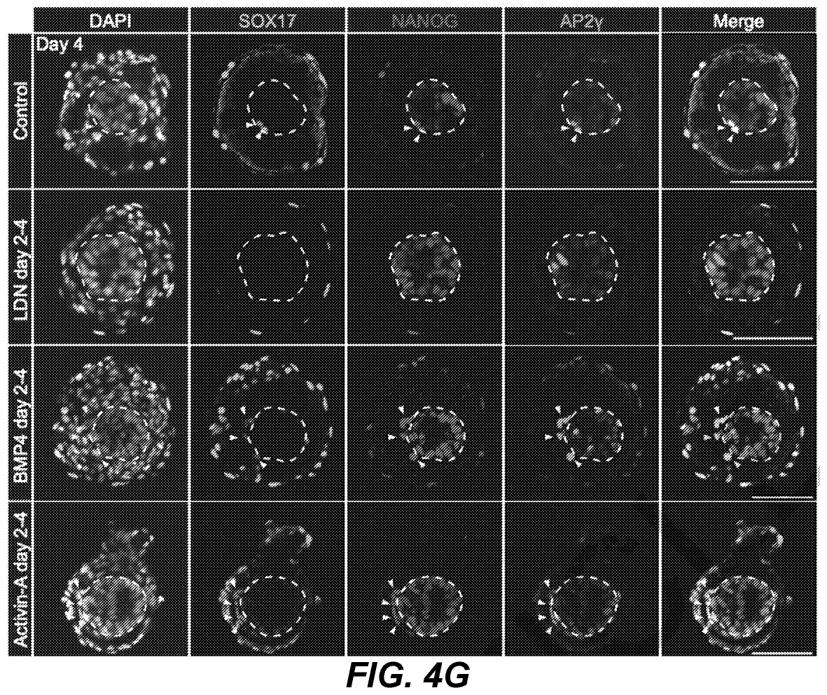

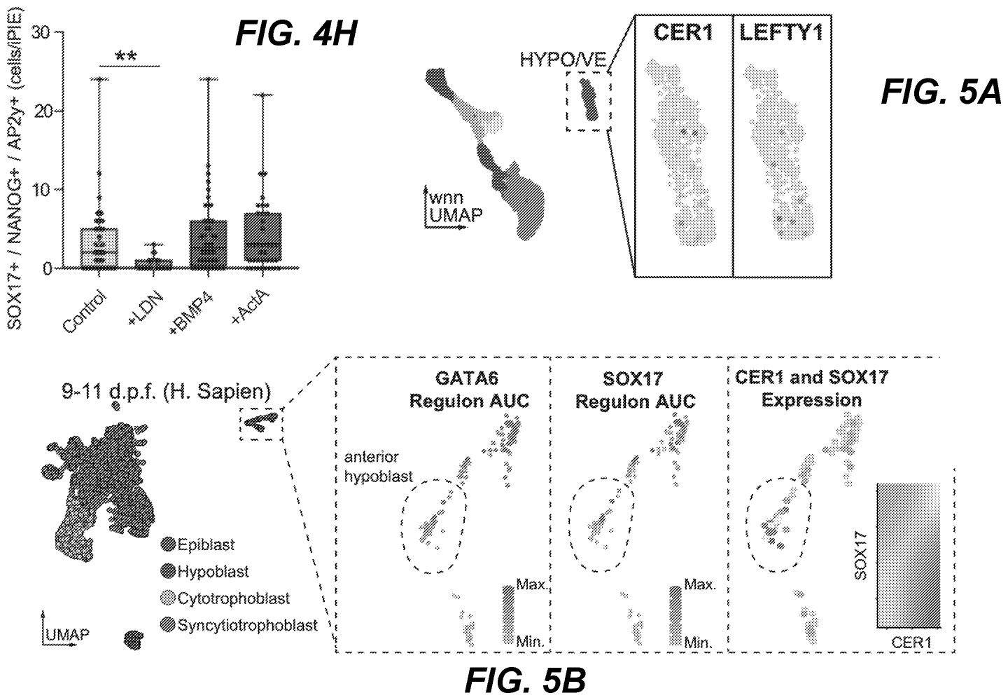

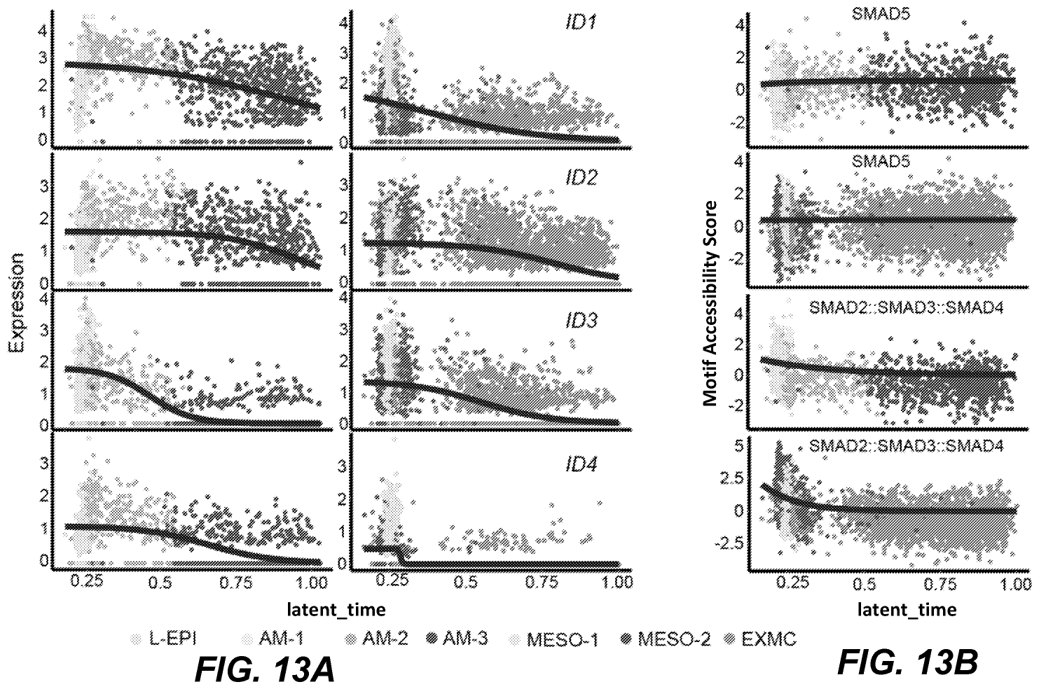

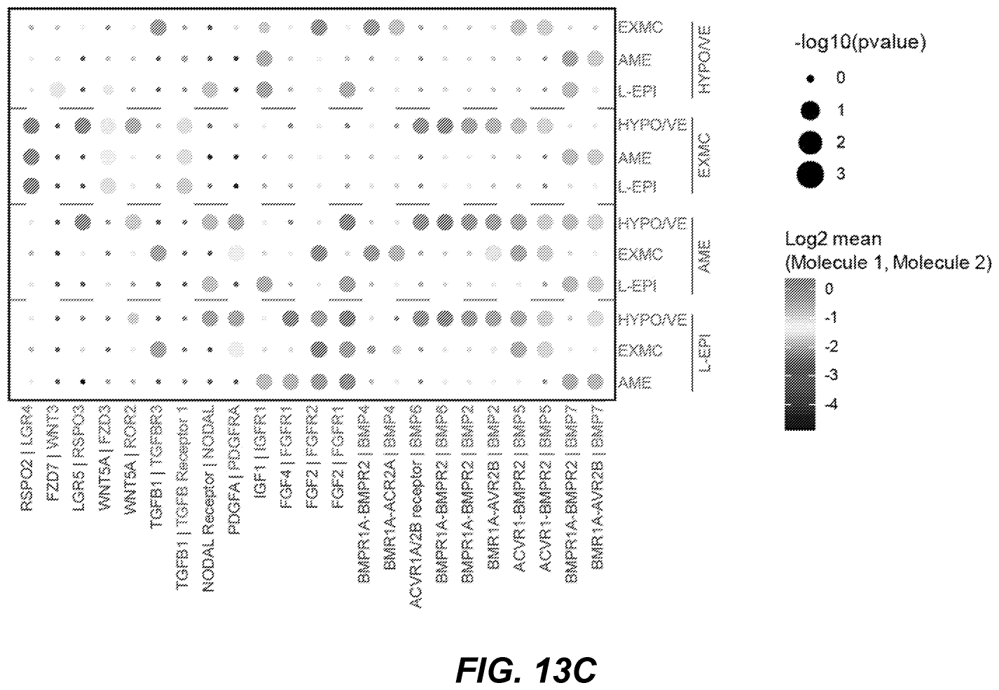

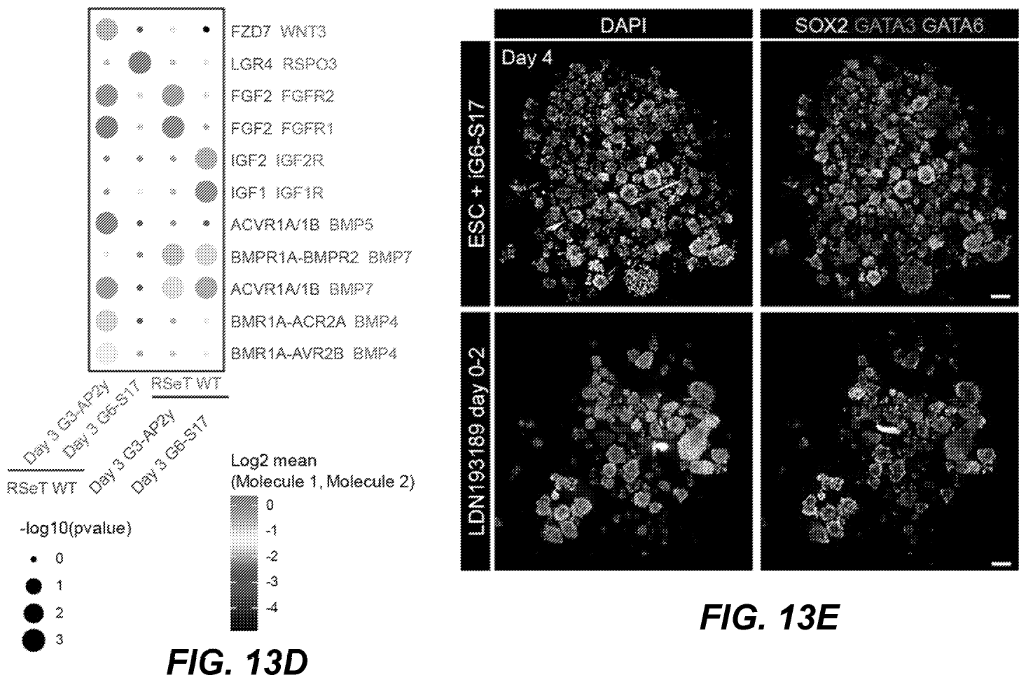

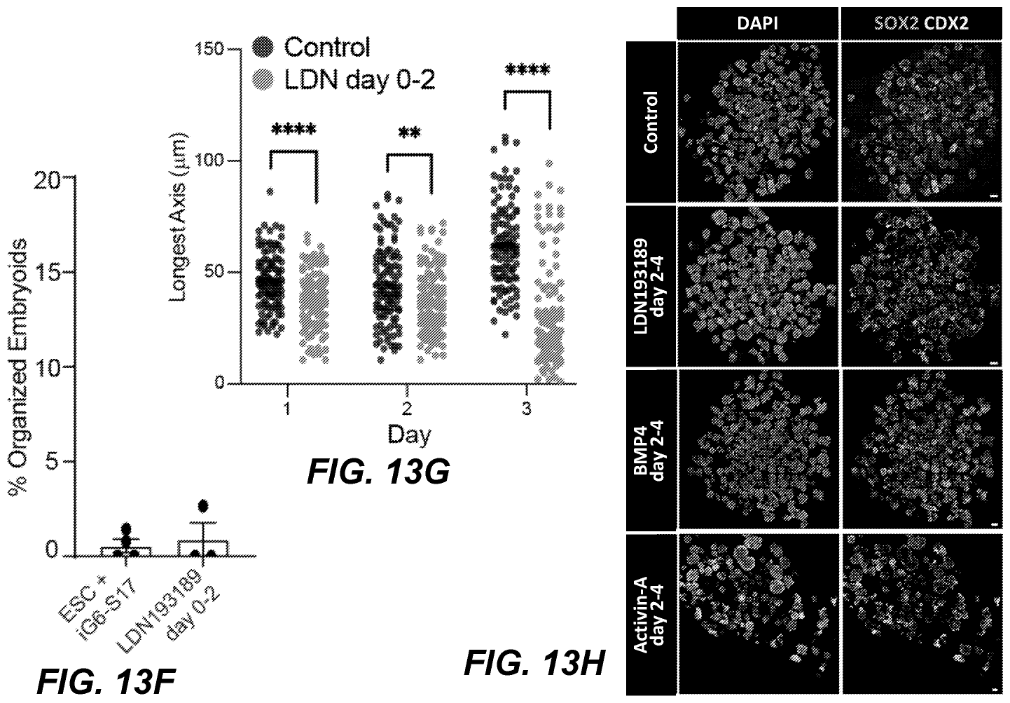

Amnion, primordial germ cells, and extraembryonic mesenchyme are thought to differentiate in response to BMP signaling in the primate embryo. To understand if this is consistent in the presently disclosed human embryo-like model, the expression of downstream BMP response genes ID1-4 was examined. ID1 and ID4 were upregulated during amnion formation, while ID2 and ID3 were enriched in both amnion and extraembryonic mesenchyme trajectories, indicating that BMP signaling was likely active (FIG. 4A and FIG. 13A). Additionally, SMAD5 motif accessibility was high in both trajectories while SMAD2::SMAD3::SMAD4 motif accessibility score, a downstream target of Activin-NODAL signaling, was not (FIG. 4B and FIG. 13B). In line with this observation, a high BMP and low NODAL signaling environment has been implicated recently in amnion differentiation of marmoset ESCs and in hESCs during extraembryonic mesenchyme differentiation, suggesting that similar dynamics may drive differentiation of these populations within inducible human embryo-like structures.

To further understand potential tissue-tissue crosstalk in the presently disclosed human embryo-like model, the computational tool CellPhoneDB was used to predict ligand-receptor pairing across clusters in single cell sequencing data (FIG. 13C-FIG. 13D). This analysis used the expression of curated receptor-ligand pairs across clusters to score potential tissue-tissue crosstalk. CellPhoneDB predicted that hypoblast cluster-derived BMP2/6 and extraembryonic mesenchyme-secreted BMP4 were likely mediators of tissue-tissue crosstalk. In contrast, predicted NODAL signaling between tissues was low, further supporting the presence of a high BMP, low NODAL signaling environment in the human embryo-like model. When CellPhoneDB was applied to the single cell sequencing data of the three cell lines aggregated to generate the embryo-like model, the inducible GATA3-AP2γ cells were predicted to be the initial source of BMP (FIG. 13D). Aggregation of inducible GATA6-SOX17 and wildtype RSeT hESC alone (i.e. without inducible GATA3-AP2γ cells) or addition of the ALK1/2/3/6 (type I BMP receptors) inhibitor LDN193189 between days 0-2 blocked formation of organized structures (FIG. 13E-FIG. 13G), demonstrating the necessity of inducible GATA3-AP2γ cell secreted BMP during embryoid formation.

To verify the role of BMP signaling during differentiation of the epiblast-like domain, phosphorylated (p)SMAD1.5 expression was examined. pSMAD1.5 enrichment in the OCT4-positive epiblast-like domain on days 4 and 6 post-aggregation was indicative of active BMP signaling (FIG. 4C). In contrast, these cells had low nuclear/cytoplasmic ratio of total SMAD2.3, reflecting low NODAL signaling within the epiblast-like domain (FIG. 4D) and in accord with the CellPhoneDB predictions. To functionally validate the role of BMP signaling in the differentiation of the inner domain, the human embryo-like model was treated with LDN193189 between 48- and 96-hours post-aggregation. Treated structures exhibited increased maintenance of SOX2 expression in the inner domain and lesser CDX2 and AP2a upregulation on Day 4 and Day 6 post-aggregation, compared to untreated control or BMP4 treated structures. Supplementation with Activin-A, an agonist of SMAD2.3 signaling, resulted in a similar phenotype, though to a lesser degree (FIG. 4E-FIG. 4F and FIG. 13H). Additionally, LDN193189 treatment decreased the number of primordial germ cell-like cells, while BMP4 or Activin-A treatment had minimal effect on the emergence of this population (FIG. 4G-FIG. 4H). These data demonstrated that endogenous BMP and NODAL were key drivers of amnion and primordial germ cell-like cell differentiation from the epiblast-like domain within inducible embryoids.

### Example 5

### SOX17 Inhibits Anterior Hypoblast

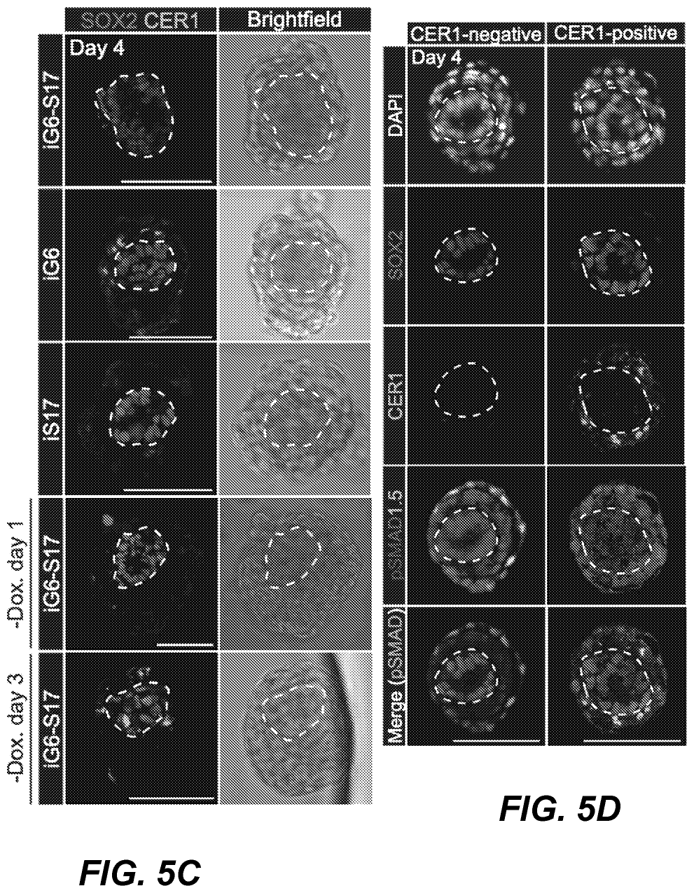

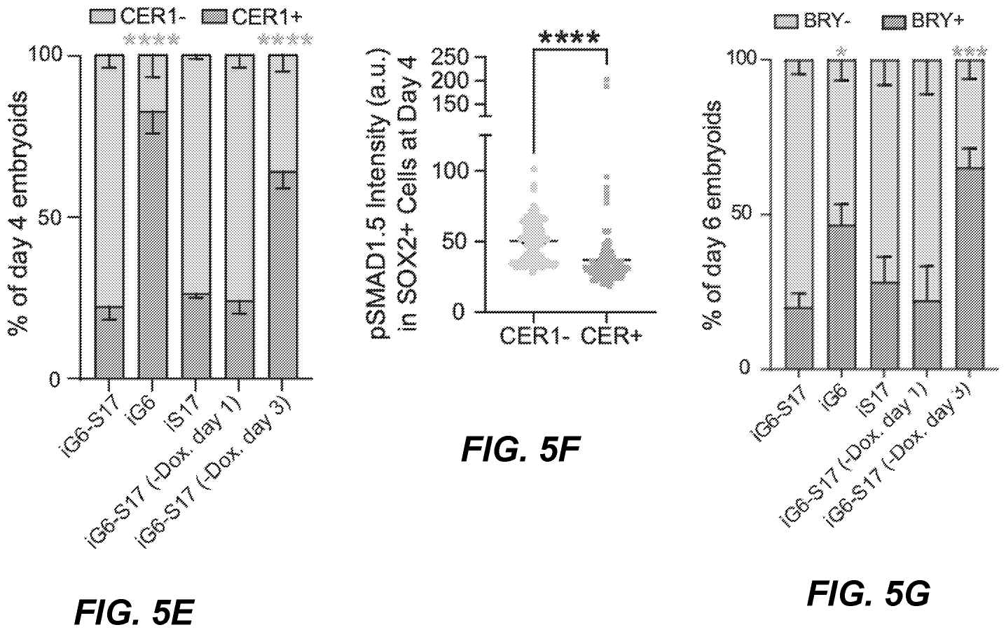

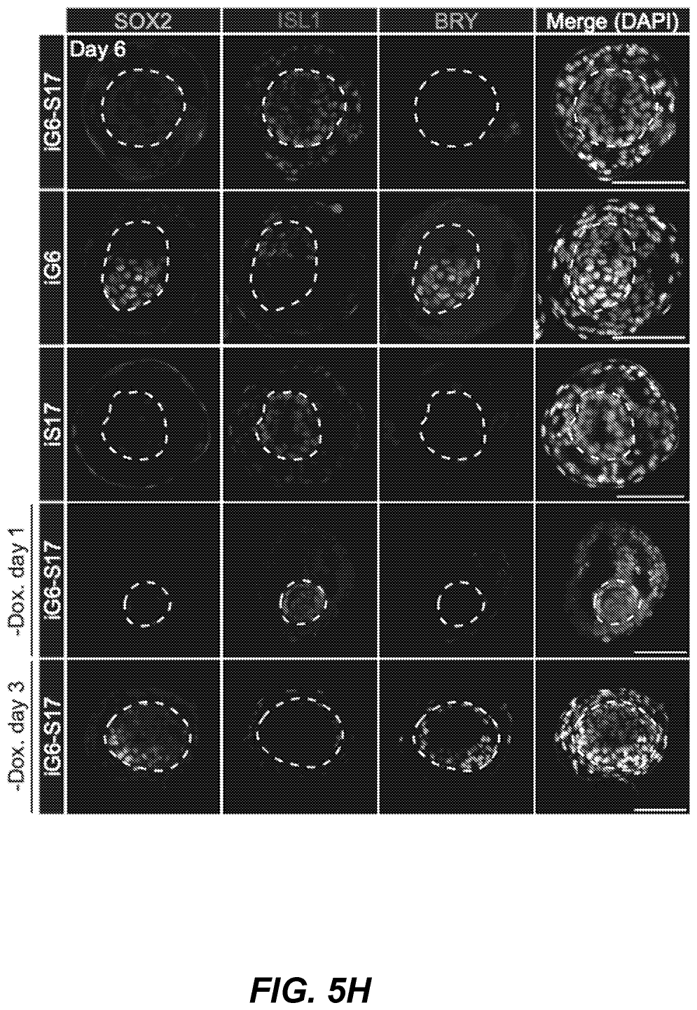

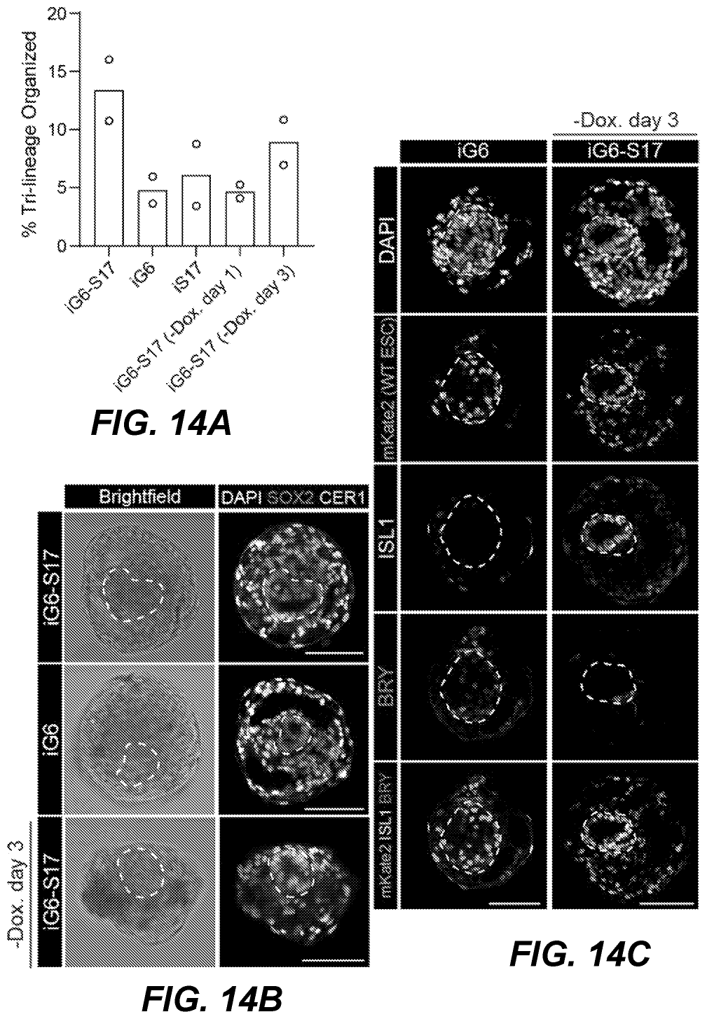

BMP signals were localized to the posterior of the embryo by the antagonistic action of the anterior hypoblast, which secreted inhibitors of BMP, WNT, and NODAL, including CER1 and LEFTY1 (FIG. 2F). It was recently demonstrated that these markers of the anterior hypoblast were expressed in peri- and post-implantation human embryos cultured in vitro. Neither CER1 nor LEFTY1 was meaningfully expressed in the HYPO/VE single cell sequencing cluster (FIG. 5A). Re-analysis of previously published 10× single cell RNA sequencing data from in vitro-cultured post-implantation human embryos revealed that SOX17 regulon activity was significantly enriched in the CER1-negative hypoblast subcluster (FIG. 5B). These sequencing data were also published in Supplementary Data Table 8 of Mole et al., “A single cell characterisation of human embryogenesis identifies pluripotency transitions and putative anterior hypoblast centre,” Nature Communications 2021, 12(1), 3679, the content of which is incorporated by reference in its entirety. Indeed, induction of SOX17 singly or in combination with GATA6 resulted in decreased capacity to upregulate CER1, as compared with GATA6 overexpression alone (FIG. 6H). To test whether single induction of GATA6 changed the identity of hypoblast-like cell subpopulations in the presently disclosed human embryo-like model, hypoblast-like cells with either inducible GATA6 or SOX17 expression alone or in combination were generated. An increased proportion of CER1-positive cells were observed within embryoids derived with inducible GATA6 cells alone compared to embryoids generated with inducible GATA6-SOX17 or SOX17 cells (FIG. 5C and FIG. 5E and FIG. 14A). To validate that SOX17 induction inhibited CER1 expression, doxycycline was withdrawn on Days 1 or 3 post-aggregation. Withdrawal on Day 3—but not Day 1—promoted CER1 expression in embryoids (FIG. 5C and FIG. 14A). Staining of pSMAD1.5 together with CER1 showed significantly decreased pSMAD1.5 expression in the inner-domain cells of structures with a CER1-positive cell population (FIG. 5D and FIG. 5F). By Day 6 post-aggregation, CER1 expression decreased in all embryoids, regardless of initial hypoblast induction regime (FIG. 14B). However, transient CER1 expression in anterior hypoblast-like cells impacted the epiblast-like domain with embryoids. Embryoids generated with single GATA6 induction or doxycycline withdrawal on Day 3 showed increased expression of primitive streak marker BRY/TBXT on Day 6 post-aggregation as compared to structures with consistent GATA6-SOX17 or SOX17 induction (FIG. 5G-FIG. 5H and FIG. 14C).

Together, these data demonstrated that functional differences in the gene regulatory network underlying differentiation of hypoblast subpopulations were observed in embryoids. An inhibitory role of prolonged SOX17 overexpression on CER1-positive anterior hypoblast identity was also shown. These experiments highlighted the value of modular embryoid models to study interactions between embryonic and extraembryonic tissues.

### Additional Consideration

In the present disclosure, a multi-lineage stem cell-derived model of the human post-implantation embryo was generated. The model undergoes epiblast-like domain lumenogenesis and differentiation and reflects developmentally relevant interactions between extraembryonic-like and embryonic-like tissues. The stem cell derived inducible model of the human embryo generates amnion-like cells in response to BMP signaling that progressively mature. Similarly, primordial germ cell-like cells readily differentiated in the stem cell model of the human embryo. Evidence that these cells were specified along the amnion differentiation trajectory, likely originating from a common AP2a-positive progenitor as reported in other in vitro systems, was presented. Extraembryonic mesenchyme-like cells, which closely resembled those of the primate embryo, were also observed. The analysis suggested a trajectory from the late epiblast-like population through a mesodermal intermediate, in line with a recently reported in vitro extraembryonic mesenchyme differentiation protocol, data from cynomolgus macaque, and historical observations in rhesus macaque and human embryos.

Unexpectedly, modulation of the transgenes that was used to drive hypoblast-like identity altered the balance of hypoblast contribution from CER1-negative to CER1-positive hypoblast-like cells. This observation demonstrated that SOX17 overexpression blocked CER1-positive anterior hypoblast formation. NODAL signaling is required for CER1-positive mouse anterior visceral endoderm formation and SOX17 may restrain excessive NODAL activity and antagonize its targets. The low NODAL activity observed in inducible human embryoids, compounded by SOX17 overexpression may, in part, explain the low levels of anterior hypoblast formation in embryoids. Additionally, the low NODAL environment, combined with lack of anterior hypoblast, could contribute to the differentiation of the epiblast-like population over time.

In the presently disclosed human embryo models with CER1-positive hypoblast, a noticeable increase in primitive streak-like BRACHYUYRY/TBXT expression was observed, despite loss of the CER1-positive cells by Day 6. Transient presence of an anterior hypoblast-like population could have protected epiblast-like domain pluripotency for a longer period, allowing cells exit pluripotency at a developmentally later, gastrulation-competent cell stage. These results contrasted with embryoids lacking an anterior hypoblast-like population, which predominantly generated amnion. These data pointed to the potential existence of a distinct intermediate pluripotent state with the ability to give rise to both amnion and extraembryonic mesenchyme but not germ layer derivatives.

The modular generation of integrated embryoids from their constitutive parts would be useful in interrogating the role of specific tissues and tissue-specific gene requirements. However, the use of transcription factor overexpression to generate extraembryonic tissues may also lead to deficiencies in differentiation. For example, while GATA3-AP2γ induction drove trophoblast-like gene programs in 2D, upon aggregation in the human embryo-like model, this cell population aberrantly upregulated endodermal markers (including SOX17 and GATA6). Nevertheless, the GATA3-AP2γ inducible cells were required for successful organization of an embryo-like structure and likely acted as a crucial source of BMP. Initial pluripotent state was a crucial factor in induction of downstream gene regulatory networks. Evidence that embryo models formed efficiently using peri-implantation stage hESCs but not more naïve hESCs was provided. However, given induction of trophoblast gene networks appeared more robust in naïve hESC, the use of discordant pluripotent generated embryo models better recapitulating the embryo. Likewise, different combinations of transcription factors could be necessary for lineage specification from different starting states. Thus, further work interrogating the epigenetic landscape and binding sites of these factors may be useful to improve strategies to generate bona fide extraembryonic cells.

In summary, a modular model of human post-implantation development that included both embryonic- and extraembryonic-like cells was presented. The post-implantation embryo model self-organized and in rare cases showed axis formation. Similar to other human integrated embryo-like models, such as blastoids, further optimization was needed to permit the maintenance of all major lineages of the post-implantation embryo with their developmental potential to a fuller extent and embryo-like morphology. As this model could not implant, it did not have the capacity to develop toward fetal stages and did not mimic stages beyond primitive streak formation. It also did not contain all cell types of the gastrulation-stage embryo. However, the construction of these integrated models of the post-implantation human embryo was an important step towards mechanistic studies of post-implantation development that were impossible to be carried out in the in vivo human embryo.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An in vitro method of generating a mammalian synthetic embryo, the method comprising:
co-culturing a wild-type mammalian embryonic stem cell (ESC), a first modified mammalian ESC comprising GATA6 gene and/or SOX17 gene, and a second modified mammalian ESC comprising GATA3 gene and/or TFAP2C gene under a condition in a culture medium allowing the ESCs to self-organize into a post-implantation embryo structure.

2. The in vitro method of claim 1, wherein the first modified mammalian ESC comprises an inducible GATA6 gene, an inducible SOX17 gene, or both, and/or wherein the second modified mammalian ESC comprises an inducible GATA3 gene, an inducible TFAP2C gene, or both.

3. (canceled)

4. (canceled)

5. (canceled)

6. The in vitro method of claim 1, further comprising contacting the first modified mammalian ESC and/or the second modified mammalian ESC with an inducer, wherein the inducer is supplied to the culture medium for a duration of about 1-7 days.

7.-10. (canceled)

11. The in vitro method of claim 1, wherein the wild type mammalian ESC and/or the modified mammalian ESCs are naïve ESCs or primed ESCs.

12. The in vitro method of claim 11, wherein the wild type mammalian ESC and/or the modified mammalian ESCs are pre-implantation naïve hESCs, peri-implantation-like pluripotent naïve hESCs, or post-implantation primed hESCs.

13. (canceled)

14. The in vitro method of claim 12, wherein the wild type mammalian ESC and the modified mammalian ESCs are peri-implantation-like pluripotent hESCs.

15. The in vitro method of claim 1, wherein the wild type mammalian ESC, the first modified mammalian ESC comprising GATA6 and/or SOX17 gene, and the second modified mammalian ESC comprising GATA3 and/or TFAP2C gene are provided at a ratio from about 1:1:1 to 1:1:5.

16. The method of claim 1, wherein the ESCs are cultured in a substrate, optionally wherein the substrate comprises a dish, a U-plate, a flask or a microwell plate.

17. (canceled)

18. (canceled)

19. The in vitro method of claim 1, wherein the co-culturing comprises co-culturing the ESCs in a stem-cell proliferation medium for about 5 days, optionally passaging the ESCs in the stem-cell proliferation medium at least two times.

20. The in vitro method of claim 19, wherein the stem-cell proliferation medium is a serum-free medium.

21. The in vitro method of claim 19, wherein the stem-cell proliferation medium comprises Dulbecco's Modified Eagle Media (DMEM), DMEM Nutrient Mixture 12 (DMEM/F12), neurobasal, N2, B27, L-glutamine or an analogue thereof, a reducing agent, an antibiotic, or a combination thereof.

22.-25. (canceled)

26. The in vitro method of claim 19, wherein the ESCs aggregate following up to 24 hours of co-culturing in the stem-cell proliferation medium.

27. (canceled)

28. The in vitro method of claim 19, wherein the co-culturing comprises co-culturing the ESCs in a post-implantation culture medium for at least 2 days, following co-culturing in the stem-cell proliferation medium.

29. The in vitro method of claim 28, wherein co-culturing the ESCs in the post-implantation culture medium begins about 2 days post-aggregation of the ESCs.

30. The in vitro method of claim 28, wherein the post-implantation culture medium comprises Dulbecco's Modified Eagle Media (DMEM), DMEM Nutrient Mixture 12 (DMEM/F12), a non-human serum or serum substitute thereof, an antibiotic, an antimicrobial agent, L-glutamine or an analogue thereof, an insulin, an insulin analogue, or an insulin receptor agonist, an estrogen analogue, or an estrogen receptor agonist, progesterone, a progesterone analogue, or a progesterone receptor agonist, or any combination thereof, wherein the non-human serum or serum substitute comprises fetal bovine serum, bovine serum albumin, KnockOut™ Serum Replacement, or any combination thereof, the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof, the estrogen receptor agonist is selected from the group comprising β-estradiol, estrone, estriol and estetrol, or any analogue thereof, the insulin receptor agonist is selected from the group comprising IGF-I, IGF-II, analogues thereof, or any combination thereof.

31.-36. (canceled)

37. The in vitro method of claim 30, wherein the post-implantation culture medium comprises DMEM/F12, fetal bovine serum, GlutaMax, non-essential amino acids, essential amino acids, Insulin-Transferrin-Selenium-Ethanolamine (ITS-X), penicillin and/or streptomycin, Glucose, sodium lactate, β-estrodiol, progesterone, or any combination thereof.

38. (canceled)

39. (canceled)

40. The in vitro method of claim 1, wherein the post-implantation embryo structure comprises an inner epiblast-like domain, a single outer layer of trophoblast-like cells, and an intermediate hypoblast-like domain between the epiblast-like domain and the single outer layer of trophoblast-like cells, and wherein the inner epiblast-like domain is SOX2 positive and contains a central lumen, the single outer layer of trophoblast-like cells is GATA3 positive, and the intermediate hypoblast-like domain is GATA6 positive.

41.-44. (canceled)

45. The in vitro method of claim 1, wherein the post-implantation embryo structure expresses TDGF1, SOX2, NANOG, TFAP2A, ID1, ISL1, TFAP2C, VTCN1, GRHBL1, MEIS1, TBXT, MESP1, MIXL1, CER1, SNAI1, EOMES, POSTN, COL6A3, IGF2, TBX20, BMP6, CDH2, HNF1B, FOXA2, or a combination thereof.

46. The in vitro method of claim 1, wherein the post-implantation embryo structure generates amnion and primordial germ cells.

47. The in vitro method of claim 1, wherein the efficiency of forming an post-implantation embryo from the wild-type mammalian ESC, the first modified mammalian ESC comprising GATA6 gene and/or SOX17 gene, and the second modified mammalian ESC comprising GATA3 gene and/or TFAP2C gene is greater than 5%, 10%, 15%, 20%, 25%, 30%, 35%, or higher.

48. The in vitro method of claim 1, wherein the method does not comprise any in vivo step, the method does not comprise culturing trophoblast stem cells, hypoblast stem cells or both, alone or in combination with the ESCs, or both.

49. (canceled)

50. (canceled)

51. The in vitro method of claim 1, wherein the wild-type mammalian ESC, the first modified mammalian ESC comprising GATA6 gene and/or SOX17 gene, and the second modified mammalian ESC comprising GATA3 gene and/or TFAP2C gene are human ESCs, and wherein the post-implantation embryo structure is a human embryo structure.

52. (canceled)

53. The in vitro method of claim 1, wherein the post-implantation embryo structure resembles a post-implantation human embryo at about 8-9 days post-fertilization, and wherein the post-implantation embryo structure comprises cell clusters resembling embryonic late-epiblast, amnion, mesoderm, extraembryonic mesenchyme, and/or hypoblast/visceral endoderm.

54. The in vitro method of claim 1, wherein the method does not comprise the use of an exogenous signaling pathway factor, or the culture medium does not comprise or is supplied with the exogenous signaling pathway factor.

55. (canceled)

56. A synthetic embryo obtained by the method of claim 1.

57.-62. (canceled)
